(19) United States
(12) Patent Application Publication
(10) Pub. No.: US 2019/0322631 A1
(43) Pub. Date: Oct. 24, 2019

(54) SUBSTITUTED OXADIAZOLES FOR COMBATING PHYTOPATHOGENIC FUNGI

(71) Applicant: BASF SE, Ludwigshafen am Rhein (DE)

(72) Inventors: Violeta TERTERYAN-SEISER, Ludwigshafen (DE); Wassilios GRAMMENOS, Ludwigshafen (DE); Maria Angelica QUINTERO PALOMAR, Limburgerhof (DE); Ian Robert CRAIG, Ludwigshafen (DE); Christine WIEBE, Ludwigshafen (DE); Tobias MENTZEL, Limburgerhof (DE); Marcus FEHR, Limburgerhof (DE); Ana ESCRIBANO CUESTA, Ludwigshafen (DE); Christian Harald WINTER, Navi Mumbai (IN); Jan Klaas LOHMANN, Ludwigshafen (DE); Bernd MUELLER, Ludwigshafen (DE); Thomas GROTE, Ludwigshafen (DE); Erica CAMBEIS, Ludwigshafen (DE)

(21) Appl. No.: 16/469,925

(22) PCT Filed: Dec. 8, 2017
(86) PCT No.: PCT/EP2017/082016

§ 371 Date: Jun. 14, 2019

(30) Foreign Application Priority Data
Dec. 19, 2016 (EP) 16205076.9
May 23, 2017 (EP) 17172465.1

(51) Int. Cl. C07D 271/06; A01N 43/82

(52) U.S. Cl. CPC C07D 271/06; A01N 43/82

(57) ABSTRACT

The present invention relates to novel trifluoromethyloxadiazoles of the formula I or an N-oxide and/or their agriculturally useful salts and to their use for controlling phytopathogenic fungi, or to a method for combating phytopathogenic harmful fungi, which process comprises treating the fungi or the materials, plants, the soil or seeds to be protected against fungal attack, with an effective amount of at least one compound of the formula I, or an N-oxide, or an agriculturally acceptable salt thereof; and to mixtures comprising at least one such compound and at least one further pesticidally active substance selected from the group consisting of herbicides, safeners, fungicides, insecticides, and plant growth regulators; and to agrochemical compositions comprising at least one compound of the formula I and to agrochemical compositions further comprising seeds.

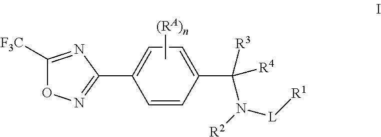

The present invention relates to novel trifluoromethyloxadiazoles of the formula I, or an N-oxide and/or their agriculturally useful salts and to their use for controlling phytopathogenic fungi, or to a method for combating phytopathogenic harmful fungi, which process comprises treating the fungi, the plants, the soil or seeds to be protected against fungal attack, with an effective amount of at least one compound of the formula I, or an N-oxide, or an agriculturally acceptable salt thereof; and to mixtures comprising at least one such compound and at least one further pesticidally active substance selected from the group consisting of herbicides, safeners, fungicides, insecticides, and plant growth regulators; and to agrochemical compositions comprising at least one compound of the formula I and to agrochemical compositions further comprising seeds.

EP 276432 A2 relates to 3-phenyl-5-trifluoromethyloxadiazole derivatives and to their use to combat phytopathogenic microorganisms. WO 2015/185485 A1 describes similar derivatives of trifluoromethyloxadiazoles and their use to combat phytopathogenic microorganisms.

R. H. Tale et al. report on anti-bacterial and anti-fungal activity of compounds bearing a trifluoromethyloxadiazole moiety (Journal of Chemical and Pharmaceutical Research 2011, 3(2), 496-505).

In many cases, in particular at low application rates, the fungicidal activity of known fungicidal compounds is unsatisfactory. Based on this, it was an objective of the present invention to provide compounds having improved activity and/or a broader activity spectrum against phytopathogenic fungi. This objective is achieved by the oxadiazoles of the formula I and/or their agriculturally useful salts for controlling phytopathogenic fungi.

The compounds described herein differ from compounds known in the prior art in the nature of the group —CR³R⁴—NR²-L-R¹, wherein the group Lisa group —S(═O)ₚ—.

Accordingly, the present invention relates to compounds of the formula I or the N-oxides, or the agriculturally acceptable salts thereof

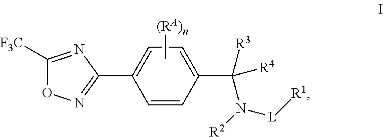

wherein:
R^{A} is independently selected from the group consisting of halogen, cyano, C₁-C₆-alkyl, C₁-C₆-haloalkyl, C₁-C₆-alkoxy or C₁-C₆-haloalkoxy;
n is 0, 1 or 2;
L is —S(═O)ₚ—;
p is 0, 1 or 2;

R¹ is C₁-C₆-alkyl; and wherein the alkyl group is substituted with 1, 2, 3, 4, 5 or up to the maximum possible number of identical or different halogen atoms; and wherein the alkyl group is further unsubstituted or, in addition to the halogen atoms, substituted with 1, 2, 3 or up to the maximum possible number of identical or different radicals selected from the group consisting of cyano, C₁-C₆-alkyl, C₁-C₆-alkoxy and C₃-C₈-cycloalkyl; and wherein the cycloalkyl group is unsubstituted or substituted with 1, 2, 3 or 4 or up to the maximum possible number of halogen atoms;
R² is hydrogen, C₁-C₆-alkyl, C₂-C₆-alkenyl, C₂-C₆-alkynyl, C₁-C₆-alkoxy, C₃-C₈-cycloalkyl, C₃-C₈-cycloalkenyl, C₃-C₈-cycloalkyl-C₁-C₆-alkyl, phenyl-C₁-C₄-alkyl, phenyl, C(═O)—(C₁-C₆-alkyl) or C(═O)—(C₁-C₆-alkoxy); and wherein any of the aliphatic or cyclic groups are unsubstituted or substituted by 1, 2, 3 or up to the maximum possible number of identical or different radicals selected from the group consisting of halogen, cyano, C₁-C₆-alkyl, C₁-C₆-alkoxy and C₃-C₈-cycloalkyl;
R³, R⁴ independently of each other are selected from the group consisting of hydrogen, halogen, cyano, C₁-C₄-alkyl, C₁-C₄-alkenyl, C₁-C₄-alkynyl, C₁-C₄-haloalkyl and C₁-C₄-alkoxy;
or R³ and R⁴ together with the carbon atom to which they are bound form a saturated 3- to 7-membered carbocycle or a saturated 3- to 6-membered heterocycle; wherein the saturated heterocycle includes beside carbon atoms 1, 2 or 3 heteroatoms independently selected from the group consisting of N, O and S as ring member atoms; and wherein said N ring member atom is substituted with the group R^{N}; and wherein
   R^{N} is hydrogen, C₁-C₆-alkyl or halogen;
   and wherein said S ring member atom is unsubstituted or substituted with 1 or 2 oxo radicals; and wherein one or two CH₂ groups of the saturated carbocycle or of the saturated heterocycle may be replaced by one or two groups independently selected from the group of —C(═O)— and —C(═S)—; and wherein the carbon ring member atoms of the saturated carbocycle or the saturated heterocycle are unsubstituted or substituted with a total number of 1, 2, 3, 4 or up to the maximum possible number of identical or different radicals selected from the group consisting of halogen, cyano, C₁-C₆-alkyl, C₁-C₆-alkoxy and C₃-C₈-cycloalkyl.

Agriculturally acceptable salts of the compounds of the formula I encompass especially the salts of those cations or the acid addition salts of those acids whose cations and anions, respectively, have no adverse effect on the fungicidal action of the compounds I. Suitable cations are thus in particular the ions of the alkali metals, preferably sodium and potassium, of the alkaline earth metals, preferably calcium, magnesium and barium, of the transition metals, preferably manganese, copper, zinc and iron, and also the ammonium ion which, if desired, may be substituted with one to four C₁-C₄-alkyl substituents and/or one phenyl or benzyl substituent, preferably diisopropylammonium, tetramethylammonium, tetrabutylammonium, trimethylbenzylammonium, furthermore phosphonium ions, sulfonium ions, preferably tri(C₁-C₄-alkyl)sulfonium, and sulfoxonium ions, preferably tri(C₁-C₄-alkyl)sulfoxonium. Anions of acceptable acid addition salts are primarily chloride, bromide, fluoride, hydrogensulfate, sulfate, dihydrogenphosphate, hydrogenphosphate, phosphate, nitrate, bicarbonate, carbonate, hexafluorosilicate, hexafluorophosphate, benzoate, and the anions of C₁-C₄-alkanoic acids, preferably formate, acetate, propionate and butyrate. They can be formed by reacting a compound I with an acid of the corresponding anion, preferably of hydrochloric acid, hydrobromic acid, sulfuric acid, phosphoric acid or nitric acid.

Compounds of the formula I can exist as one or more stereoisomers. The various stereoisomers include enantiomers, diastereomers, atropisomers arising from restricted rotation about a single bond of asymmetric groups and geometric isomers. They also form part of the subject matter of the present invention. One skilled in the art will appreciate that one stereoisomer may be more active and/or may exhibit beneficial effects when enriched relative to the other stereoisomer(s) or when separated from the other stereoisomer(s). Additionally, the skilled artisan knows how to separate, enrich, and/or to selectively prepare said stereoisomers. The compounds of the invention may be present as a mixture of stereoisomers, e.g. a racemate, individual stereoisomers, or as an optically active form.

Compounds of the formula I can be present in different crystal modifications whose biological activity may differ. They also form part of the subject matter of the present invention.

In respect of the variables, the embodiments of the intermediates obtained during preparation of compounds I correspond to the embodiments of the compounds of formula I. The term “compounds I” refers to compounds of formula I.

In the definitions of the variables given above, collective terms are used which are generally representative for the substituents in question. The term “Cₙ-Cₘ” indicates the number of carbon atoms possible in each case in the substituent or substituent moiety in question.

The moieties having two ore more possibilities to be attached apply following:

The moieties having no brackets in the name are bonded via the last moiety e.g. heteroaryl-C₁-C₄-alkyl is bonded via C₁-C₄-alkyl. etc.

The term “halogen” refers to fluorine, chlorine, bromine and iodine.

The term “C₁-C₆-alkyl” refers to a straight-chained or branched saturated hydrocarbon group having 1 to 6 carbon atoms, for example methyl, ethyl, propyl, 1-methylethyl, butyl, 1-methylpropyl, 2-methylpropyl, and 1,1-dimethylethyl.

The term “C₁-C₆-haloalkyl” refers to a straight-chained or branched alkyl group having 1 to 6 carbon atoms (as defined above), wherein some or all of the hydrogen atoms in these groups may be replaced by halogen atoms as mentioned above, for example chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl and pentafluoroethyl, 2-fluoropropyl, 3-fluoropropyl, 2,2-difluoropropyl, 2,3-difluoropropyl, 2-chloropropyl, 3-chloropropyl, 2,3-dichloropropyl, 2-bromopropyl, 3-bromopropyl, 3,3,3-trifluoropropyl, 3,3,3-trichloropropyl, CH₂—C₂F₅, CF₂—C₂F₅, CF(CF₃)₂, 1-(fluoromethyl)-2-fluoroethyl, 1-(chloromethyl)-2-chloroethyl, 1-(bromomethyl)-2-bromoethyl, 4-fluorobutyl, 4-chlorobutyl, 4-bromobutyl or nonafluorobutyl.

The term “C₁-C₆-alkoxy” refers to a straight-chain or branched alkyl group having 1 to 6 carbon atoms (as defined above) which is bonded via an oxygen, at any position in the alkyl group, for example methoxy, ethoxy, n-propoxy, 1-methylethoxy, butoxy, 1-methylpropoxy, 2-methylpropoxy or 1,1-dimethylethoxy.

The term “phenyl-C₁-C₄-alkyl” refers to alkyl having 1 to 4 carbon atoms (as defined above), wherein one hydrogen atom of the alkyl radical is replaced by a phenyl radical.

The term “C₂-C₆-alkenyl” refers to a straight-chain or branched unsaturated hydrocarbon radical having 2 to 6 carbon atoms and a double bond in any position, such as ethenyl, 1-propenyl, 2-propenyl (allyl), 1-methylethenyl, 1-butenyl, 2-butenyl, 3-butenyl, 1-methyl-1-propenyl, 2-methyl-1-propenyl, 1-methyl-2-propenyl, 2-methyl-2-propenyl.

The term “C₂-C₆-alkynyl” refers to a straight-chain or branched unsaturated hydrocarbon radical having 2 to 6 carbon atoms and containing at least one triple bond, such as ethynyl, 1-propynyl, 2-propynyl (propargyl), 1-butynyl, 2-butynyl, 3-butynyl, 1-methyl-2-propynyl.

The term “C₃-C₈-cycloalkyl” refers to monocyclic saturated hydrocarbon radicals having 3 to 8 carbon ring members such as cyclopropyl (C₃H₅), cyclobutyl, cyclopentyl, cyclohexyl, cycloheptyl or cyclooctyl.

The terms “C(═O)—(C₁-C₄-alkyl) or C(═O)—(C₁-C₄-alkoxy)” refers to a radical which is attached through the carbon atom of the —C(═O)— group as indicated by the number valence of the carbon atom.

The term “aliphatic” refers to compounds or radicals composed of carbon and hydrogen atoms and which are non-aromatic compounds. An “alicyclic” compound or radical is an organic compound that is both aliphatic and cyclic. They contain one or more all-carbon rings which may be either saturated or unsaturated, but do not have aromatic character.

The terms “cyclic moiety” or “cyclic group”refer to a radical which is an alicyclic ring or an aromatic ring, such as, for example, phenyl or heteroaryl.

The term “and wherein any of the aliphatic or cyclic groups are unsubstituted or substituted with . . . ” refers to aliphatic groups, cyclic groups and groups, which contain an aliphatic and a cyclic moiety in one group, such as in, for example, phenyl-C₁-C₄-alkyl; therefore a group which contains an aliphatic and a cyclic moiety both of these moieties may be substituted or unsubstituted independently of each other.

The term “phenyl” refers to an aromatic ring systems including six carbon atoms (commonly referred to as benzene ring

The term “saturated 3- to 7-membered carbocycle” is to be understood as meaning saturated carbocycles having 3, 4, 5, 6 or 7 ring members. Examples include cyclopropyl, cyclopentyl, cyclopentenyl, cyclopentadienyl, cyclohexyl, cyclohexenyl, cyclohexadienyl, cycloheptyl, cycloheptenyl, cycloheptadienyl, and the like.

The term “saturated 3- to 6-membered heterocycle, wherein the saturated heterocycle includes besides carbon atoms further 1, 2 or 3 heteroatoms selected from N, O and S as ring member atoms”, is to be understood as meaning, for example:

a 3- or 4-membered saturated heterocycle which contains 1 or 2 heteroatoms from the group consisting of N, O and S as ring members, such as oxirane, aziridine, thiirane, oxetane, azetidine, thiethane, [1,2]dioxetane, [1,2]dithietane, [1,2]diazetidine; and a 5- or 6-membered saturated heterocycle which contains 1, 2 or 3 heteroatoms from the group consisting of N, O and S as ring members such as 2-tetrahydrofuranyl, 3-tetrahydrofuranyl, 2-tetrahydrothienyl, 3-tetrahydrothienyl, 2-pyrrolidinyl, 3-pyrrolidinyl, 3-isoxazolidinyl, 4-isoxazolidinyl, 5-isoxazolidinyl, 3-isothiazolidinyl, 4-isothiazolidinyl, 5-isothiazolidinyl, 3-pyrazolidinyl, 4-pyrazolidinyl, 5-pyrazolidinyl, 2-oxazolidinyl, 4-oxazolidinyl, 5-oxazolidinyl, 2-thiazolidinyl, 4-thiazolidinyl, 5-thiazolidinyl, 2-imidazolidinyl, 4-imidazolidinyl, 1,2,4-oxadiazolidin-3-yl, 1,2,4-oxadiazolidin-5-yl, 1,2,4-thiadiazolidin-3-yl, 1,2,4-thiadiazolidin-5-yl, 1,2,4-triazolidin-3-yl, 1,3,4-oxadiazolidin-2-yl, 1,3,4-thiadiazolidin-2-yl, 1,3,4-triazolidin-2-yl, 2,3-dihydrofur-2-yl, 2,3-dihydrofur-3-yl, 2,4-dihydrofur-2-yl, 2,4-dihydrofur-3-yl, 2,3-dihydrothien-2-yl, 2,3-dihydrothien-3-yl, 2,4-dihydrothien-2-yl, 2,4-dihydrothien-3-yl, 2-pyrrolin-2-yl, 2-pyrrolin-3-yl, 3-pyrrolin-2-yl, 3-pyrrolin-3-yl, 2-isoxazolin-3-yl, 3-isoxazolin-3-yl, 4-isoxazolin-3-yl, 2-isoxazolin-4-yl, 3-isoxazolin-4-yl, 4-isoxazolin-4-yl, 2-isoxazolin-5-yl, 3-isoxazolin-5-yl, 4-isoxazolin-5-yl, 2-isothiazolin-3-yl, 3-isothiazolin-3-yl, 4-isothiazolin-3-yl, 2-isothiazolin-4-yl, 3-isothiazolin-4-yl, 4-isothiazolin-4-yl, 2-isothiazolin-5-yl, 3-isothiazolin-5-yl, 4-isothiazolin-5-yl, 2,3-dihydropyrazol-1-yl, 2,3-dihydropyrazol-2-yl, 2,3-dihydropyrazol-3-yl, 2,3-dihydropyrazol-4-yl, 2,3-dihydropyrazol-5-yl, 3,4-dihydropyrazol-1-yl, 3,4-dihydropyrazol-3-yl, 3,4-dihydropyrazol-4-yl, 3,4-dihydropyrazol-5-yl, 4,5-dihydropyrazol-1-yl, 4,5-dihydropyrazol-3-yl, 4,5-dihydropyrazol-4-yl, 4,5-dihydropyrazol-5-yl, 2,3-dihydrooxazol-2-yl, 2,3-dihydrooxazol-3-yl, 2,3-dihydrooxazol-4-yl, 2,3-dihydrooxazol-5-yl, 3,4-dihydrooxazol-2-yl, 3,4-dihydrooxazol-3-yl, 3,4-dihydrooxazol-4-yl, 3,4-dihydrooxazol-5-yl, 3,4-dihydrooxazol-2-yl, 3,4-dihydrooxazol-3-yl, 3,4-dihydrooxazol-4-yl, 2-piperidinyl, 3-piperidinyl, 4-piperidinyl, 1,3-dioxan-5-yl, 2-tetrahydropyranyl, 4-tetrahydropyranyl, 2-tetrahydrothienyl, 3-hexahydropyridazinyl, 4-hexahydropyridazinyl, 2-hexahydropyrimidinyl, 4-hexahydropyrimidinyl, 5-hexahydropyrimidinyl, 2-piperazinyl, 1,3,5-hexahydrotriazin-2-yl and 1,2,4-hexahydrotriazin-3-yl and also the corresponding -ylidene radicals; and

a 7-membered saturated or partially unsaturated heterocycle such as tetra- and hexahydroazepinyl, such as 2,3,4,5-tetrahydro[1H]azepin-1-,-2-,-3-,-4-,-5-,-6- or-7-yl, 3,4,5,6-tetrahydro[2H]azepin-2-,-3-,-4-,-5-,-6- or-7-yl, 2,3,4,7-tetrahydro[1H]azepin-1-,-2-,-3-,-4-,-5-,-6-or-7-yl, 2,3,6,7-tetrahydro[1H]azepin-1-,-2-,-3-,-4-,-5-,-6- or-7-yl, hexahydroazepin-1-,-2-,-3- or-4-yl, tetra- and hexahydrooxepinyl such as 2,3,4,5-tetrahydro[1H]oxepin-2-,-3-,-4-,-5-,-6- or-7-yl, 2,3,4,7-tetrahydro[1H]oxepin-2-,-3-,-4-,-5-,-6- or-7-yl, 2,3,6,7-tetrahydro[1H]oxepin-2-, -3-,-4-,-5-,-6- or-7-yl, hexahydroazepin-1-,-2-,-3- or-4-yl, tetra- and hexahydro-1,3-diazepinyl, tetra- and hexahydro-1,4-diazepinyl, tetra- and hexahydro-1,3-oxazepinyl, tetra- and hexahydro-1,4-oxazepinyl, tetra- and hexahydro-1,3-dioxepinyl, tetra- and hexahydro-1,4-dioxepinyl and the corresponding -ylidene radicals.

In respect of the variables, the embodiments of the intermediates correspond to the embodiments of the compounds I. Preference is given to those compounds I and, where applicable, also to compounds of all subformulae provided herein, e. g. formulae I.A to I.L, wherein variables R¹, R², R³, R⁴, L, p, R^{A} and n have independently of each other or more preferably in combination (any possible combination of 2 or more substituents as defined herein) the following meanings:

In a preferred embodiment R^{A} is independently selected from the group consisting of halogen, C₁-C₆-alkyl or C₃-C₈-cycloalkyl. In another preferred embodiment R^{A} is independently selected from the group consisting of halogen, methyl or ethyl. More preferably R^{A} is independently selected from the group consisting of halogen, in particular R^{A} is fluorine.

In one aspect of the invention n is 0, 1 or 2, preferably n is 1. In a particularly preferred aspect n is 0.

In one aspect of the invention L is —S(═O)ₚ—, wherein p is 1 or 2; preferably p is 2.

In one embodiment R¹ is C₁-C₆-alkyl; and wherein the alkyl group is substituted with 1, 2, 3, 4, 5 or up to the maximum possible number of identical or different halogen atoms; and wherein the alkyl group is further unsubstituted or, in addition to the halogen atoms, substituted with 1, 2, 3 or up to the maximum possible number of identical or different groups selected from the group consisting of C₁-C₆-alkyl or C₃-C₈-cycloalkyl; and wherein the cycloalkyl group is unsubstituted or substituted with 1, 2, 3 or 4 or up to the maximum possible number of radicals selected from the group consisting of chlorine and fluorine.

In one embodiment R¹ is C₃-C₈-cycloalkyl-C₁-C₂-alkyl; and wherein any of the alkyl or cycloalkyl groups are unsubstituted or substituted with 1, 2, 3 or 4 or up to the maximum possible number of radicals selected from the group consisting of chlorine and fluorine.

In one embodiment R¹ is cyclopropyl-C₁-C₂-alkyl or cyclobutyl-C₁-C₂-alkyl; and wherein any of the alkyl or cycloalkyl groups are unsubstituted or substituted with 1, 2, 3 or 4 or up to the maximum possible number of fluorine atoms.

In a further embodiment R¹ is difluoromethyl, trifluoromethyl, 2,2-difluoroethyl, 2,2,2-trifluoro-ethyl, 2-chloro-2-fluoroethyl, 2-chloro-2,2-difluoroethyl, 2,2,2-trichloroethyl and pentafluoroethyl, 3,3,3-trifluoropropyl, CH₂CF₂CF₃ or CF₂CF₂CF₅, CH(CH₃)CF₃, CH₂CF₂CH₃, CH₂C(CH₃)₂F, CH₂CH(CH₃)CF₃, CH₂C(CH₃)₂CF₃ or 2,2-difluorocycloproplymethyl.

In a preferred embodiment R¹ is 2,2,2-trifluoroethyl.

In another preferred embodiment R¹ is 2,2-difluorocycloproplymethyl.

In one aspect of the invention R² is hydrogen, C₁-C₆-alkyl, C₁-C₆-alkoxy, C₂-C₆-alkenyl, ethynyl, propargyl, C₃-C₈-cycloalkyl, C₃-C₈-cycloalkenyl or phenyl; and wherein any of the aliphatic or cyclic groups are unsubstituted or substituted with 1, 2, 3, 4 or up to the maximum possible number of identical or different radicals selected from the group consisting of halogen, cyano, C₁-C₆-alkyl, C₁-C₆-alkoxy and C₃-C₈-cycloalkyl; more preferably from halogen, in particular the radical is fluorine.

In a preferred aspect of the invention R² is hydrogen, C₁-C₆-alkyl, C₁-C₆-alkenyl, ethynyl, propargyl, C₃-C₈-cycloalkyl, C₃-C₈-cycloalkyl-C₁-C₄-alkyl or phenyl; and wherein any of the aliphatic or cyclic groups are unsubstituted or substituted with 1, 2, 3, 4 or up to the maximum possible number of identical or different radicals selected from the group consisting of halogen or C₁-C₆-alkyl, in particular fluorine.

In one further aspect R² is hydrogen, C₁-C₆-alkyl, C₁-C₆-alkenyl, C₃-C₈-cycloalkyl, C₃-C₈-cycloalkyl-C₁-C₄-alkyl or phenyl; wherein the phenyl group is unsubstituted or substituted with 1, 2, 3, 4 or up to the maximum possible number of identical or different halogen atoms.

In yet another aspect R² is hydrogen, methyl, ethyl, iso-propyl, cyclopropyl, allyl, phenyl, 4-F-phenyl or 2-F-phenyl.

In a more preferred aspect of the invention R² is hydrogen, methyl, ethyl, iso-propyl, cyclopropyl or allyl.

In a particularly preferred aspect R² is hydrogen, methy, ethyl or cyclopropyl.

In a preferred aspect of the invention R² is hydrogen, C₁-C₆-alkyl, C₁-C₆-alkenyl, ethynyl, propargyl, C₃-C₈-cycloalkyl, C₃-C₈-cycloalkyl-C₁-C₄-alkyl or phenyl; and wherein any of the aliphatic or cyclic groups are unsubstituted or substituted with 1, 2, 3, 4 or up to the maximum possible number of identical or different radicals selected from the group consisting of halogen or C₁-C₆-alkyl, in particular fluorine; and R¹ is C₁-C₆-alkyl; and wherein the alkyl group is substituted with 1, 2, 3, 4, 5 or up to the maximum possible number of identical or different halogen atoms; and wherein the alkyl group is further unsubstituted or substituted with 1, 2, 3 or up to the maximum possible number of identical or different groups selected from the group consisting of C₁-C₆-alkyl or C₃-C₈-cycloalkyl; and wherein the cycloalkyl group in R¹ is unsubstituted or substituted with 1, 2, 3 or 4 or up to the maximum possible number of radicals selected from the group consisting of chlorine and fluorine.

In a further embodiment R² is hydrogen, C₁-C₆-alkyl, C₁-C₆-alkenyl, C₃-C₈-cycloalkyl, C₃-C₈-cycloalkyl-C₁-C₄-alkyl; preferably hydrogen, methyl, ethyl, iso-propyl, cyclopropyl, CH₂-cyclopropyl or allyl; and R¹ is C₁-C₆-alkyl; and wherein the alkyl group is substituted with 1, 2, 3, 4, 5 or up to the maximum possible number of identical or different halogen atoms; and wherein the alkyl group is further unsubstituted or substituted with 1, 2, 3 or up to the maximum possible number of identical or different groups selected from the group consisting of C₁-C₆-alkyl or C₃-C₈-cycloalkyl; and wherein the cycloalkyl group in R¹ is unsubstituted or substituted with 1, 2, 3 or 4 or up to the maximum possible number of radicals selected from the group consisting of chlorine and fluorine.

In one embodiment R² is hydrogen, C₁-C₆-alkyl, C₂-C₆-alkenyl or C₃-C₈-cycloalkyl and R¹ is C₁-C₆-alkyl; and wherein the alkyl group in R¹ is substituted with 1, 2, 3 or up to the maximum possible number of atoms selected from the group consisting of fluorine and chlorine.

In another embodiment R² is hydrogen, C₁-C₆-alkyl, C₁-C₆-alkenyl, C₃-C₈-cycloalkyl, C₃-C₈-cycloalkyl-C₁-C₄-alkyl; preferably hydrogen, methyl, ethyl, iso-propyl, cyclopropyl, CH₂-cyclopropyl or allyl; and R¹ is difluoromethyl, trifluoromethyl, 2,2-difluoroethyl, 2,2,2-trifluoro-ethyl, 2-chloro-2-fluoroethyl, 2-chloro-2,2-difluoroethyl, 2,2,2-trichloroethyl and pentafluoroethyl, 3,3,3-trifluoropropyl, CH₂CF₂CF₃ or CF₂CF₂CF₅, CH(CH₃)CF₃, CH₂CF₂CH₃, CH₂C(CH₃)₂F, CH₂CH(CH₃)CF₃ or CH₂C(CH₃)₂CF₃; in particular R¹ is 2,2,2-trifluoroethyl.

In another embodiment R² is hydrogen, C₁-C₆-alkyl, C₁-C₆-alkenyl, C₃-C₈-cycloalkyl, C₃-C₈-cycloalkyl-C₁-C₄-alkyl; preferably hydrogen, methyl, ethyl, iso-propyl, cyclopropyl, CH₂-cyclopropyl or allyl; and R¹ is C₃-C₈-cycloalkyl-C₁-C₂-alkyl; and wherein any of the alkyl or cycloalkyl groups in R¹ are unsubstituted or substituted with 1, 2, 3 or 4 or up to the maximum possible number of radicals selected from the group consisting of chlorine and fluorine.

In another embodiment R² is hydrogen, C₁-C₆-alkyl, C₁-C₆-alkenyl, C₃-C₈-cycloalkyl, C₃-C₈-cycloalkyl-C₁-C₄-alkyl; preferably hydrogen, methyl, ethyl, iso-propyl, cyclopropyl, CH₂-cyclopropyl or allyl; and R¹ is cyclopropyl-C₁-C₂-alkyl or cyclobutyl-C₁-C₂-alkyl; and wherein any of the alkyl or cycloalkyl groups in R¹ are unsubstituted or substituted with 1, 2, 3 or 4 or up to the maximum possible number of fluorine atoms.

In another embodiment R² is hydrogen, C₁-C₆-alkyl, C₁-C₆-alkenyl, C₃-C₈-cycloalkyl, C₃-C₈-cycloalkyl-C₁-C₄-alkyl; preferably hydrogen, methyl, ethyl, iso-propyl, cyclopropyl, CH₂-cyclopropyl or allyl; and R¹ is 2,2-difluorocycloproplymethyl.

In one embodiment the invention relates to compounds of the formula I, wherein R³ and R⁴ independently of each other are hydrogen, halogen, C₁-C₆-alkyl or C₁-C₆-haloalkyl.

In a further embodiment R³ and R⁴ are independently of each other hydrogen, fluorine, methyl or trifluoromethyl.

In another aspect R³ and R⁴ are both hydrogen.

In a further aspect R³ is hydrogen and R⁴ is methyl.

In yet another aspect R³and R⁴ are both methyl.

In a further aspect R³ and R⁴ are both fluorine.

In one embodiment R³and R⁴ are both trifluoromethyl.

In one embodiment R³ and R⁴ together with the carbon atom to which they are bound form a vinyl group or a saturated monocyclic 3- to 5-membered saturated heterocycle or saturated carbocycle; and wherein the saturated heterocycle includes beside one or more carbon atoms no heteroatoms or 1 or 2 heteroatoms independently selected from N, O and S as ring member atoms; and wherein the vinyl group, the heterocycle or the carbocycle is unsubstituted or substituted 1, 2, 3, 4 or up to the maximum possible number of identical or different radicals selected from the group consisting of halogen, cyano and C₁-C₂-alkyl.

In another embodiment R³ and R⁴ together with the carbon atom to which they are bound form a vinyl group or a 3- or 4-membered carbocylic ring; and wherein the vinyl group or the carbocylic ring is unsubstituted.

In another aspect R³ and R⁴ together with the carbon atom to which they are bound form vinyl group or a cyclopropyl group, wherein the vinyl group or the cyclopropyl group is unsubstituted.

In another aspect R³ and R⁴ together with the carbon atom to which they are bound form a cyclopropyl group, wherein the cyclopropyl group is unsubstituted.

In still another embodiment R³ and R⁴ together with the carbon atom to which they are bound form a saturated 3-membered heterocycle; wherein the heterocycle includes beside two carbon atoms one heteroatom selected from N, O and S as ring member atoms; and wherein the heterocycle is unsubstituted.

In a further embodiment the invention relates to compounds (of formula I, or the N-oxides, or the agriculturally acceptable salts thereof, wherein:
R^{A} is independently selected from the group consisting of halogen, cyano, C₁-C₆-alkyl, C₁-C₆-haloalkyl, C₁-C₆-alkoxy or C₁-C₆-haloalkoxy;
n is 0, 1 or 2;
L is —S(═O)ₚ—;
p is 0, 1 or 2;
R¹ is C₁-C₆-alkyl; and wherein the alkyl group is substituted with 1, 2, 3, 4, 5 or up to the maximum possible number of identical or different halogen atoms; and wherein the alkyl group is further unsubstituted or, in addition to the halogen atoms, substituted with 1, 2, 3 or up to the maximum possible number of identical or different groups selected from the group consisting of C₁-C₆-alkyl or C₃-C₈-cycloalkyl; and wherein the cycloalkyl group is unsubstituted or substituted with 1, 2, 3 or 4 or up to the maximum possible number of radicals selected from the group consisting of chlorine and fluorine;
R² is hydrogen, C₁-C₆-alkyl, C₁-C₆-alkenyl, C₃-C₈-cycloalkyl, C₃-C₈-cycloalkyl-C₁-C₄-alkyl or phenyl; wherein the phenyl group is unsubstituted or substituted with 1, 2, 3, 4 or up to the maximum possible number of identical or different halogen atoms;
R³, R⁴ independently of each other are hydrogen, halogen, cyano, C₁-C₆-alkyl or C₁-C₆-haloalkyl; particularly both are hydrogen; or
R³ and R⁴ together with the carbon atom to which they are bound form a vinyl group or a saturated monocyclic 3- to 5-membered heterocycle or carbocycle, wherein the heterocycle includes beside carbon atoms 1 or 2 heteroatoms independently selected from N, O and S as ring member atoms; and wherein the vinyl group, the heterocycle or the carbocycle is unsubstituted or substituted with 1, 2, 3, 4 or up to the maximum possible number of identical or different radicals selected from the group consisting of halogen, cyano or C₁-C₂-alkyl.

In a further embodiment the invention relates to compounds (I.1), wherein n is 0, and wherein L is —S(═O)₂—.

In a further embodiment the invention relates to compounds of formula I, or the N-oxides, or the agriculturally acceptable salts thereof, wherein:
n is 0;
L is —S(═O)ₚ—;
p is 0, 1 or 2;
R¹ is difluoromethyl, trifluoromethyl, 2,2-difluoroethyl, 2,2,2-trifluoroethyl, 2-chloro-2-fluoroethyl, 2-chloro-2,2-difluoroethyl, 2,2,2-trichloroethyl and pentafluoroethyl, 3,3,3-trifluoropropyl, CH₂CF₂CF₃ or CF₂CF₂CF₅, CH(CH₃)CF₃, CH₂CF₂CH₃, CH₂C(CH₃)₂F, CH₂CH(CH₃)CF₃, CH₂C(CH₃)₂CF₃ or 2,2-difluorocycloproplymethyl;
R² is hydrogen, methyl, ethyl, n-propyl, iso-propyl, cyclopropyl, allyl, phenyl, 4-F-phenyl, or 2-F-phenyl;
R³ and R⁴ independently of each other are hydrogen, fluorine, methyl or trifluoromethyl; particularly both are hydrogen; or
R³ and R⁴ together with the carbon atom to which they are bound form a 3- or 4-membered carbocylic ring and wherein the carbocylic ring is unsubstituted; or R³ and R⁴ together with the carbon atom to which they are bound form a saturated 3-membered heterocycle; wherein the heterocycle includes beside two carbon atoms one heteroatom selected from N, O and S as ring member atoms; and wherein the heterocycle is unsubstituted.

Compounds of the formulae I.A, I.B, I.C, I.D, I.E are useful for controlling phytopathogenic fungi, wherein the variables R¹ and R² are as defined or preferably defined herein. According to one embodiment, the present invention relates to compounds of the formulae I.F, I.G, I.H, I.J, I.K and I.L and to their use for controlling phytopathogenic fungi, wherein the variables R¹ and R² are as defined or preferably defined herein.

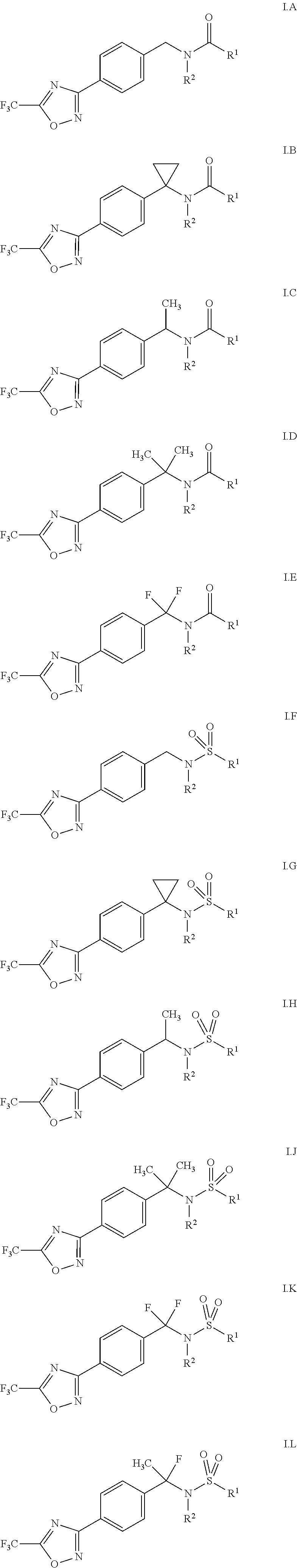

Preference is given to the compounds I used according to the invention and to the compounds according to the invention compiled in Tables 1 to 11 below. The groups mentioned for a substituent in the tables are furthermore per se, independently of the combination in which they are mentioned, a particularly preferred aspect of the substituent in question.

Table 1: Compounds of the formula I.A, in which R¹ and R² for each individual compound corresponds in each case to one line A-1 to A-323 of Table A (compounds I.A.A-1 to I.A.A-323).

Table 2: Compounds of the formula I.B, in which R¹ and R² for each individual compound corresponds in each case to one line A-1 to A-323 of Table A (compounds I.B.A-1 to I.B.A-323).

Table 3: Compounds of the formula I.C, in which R¹ and R² for each individual compound corresponds in each case to one line A-1 to A-323 of Table A (compounds I.C.A-1 to I.C.A-323)

Table 4: Compounds of the formula I.D, in which R¹ and R² for each individual compound corresponds in each case to one line A-1 to A-323 of Table A (compounds I.D.A-1 to I.D.A-323).

Table 5: Compounds of the formula I.E, in which R¹ and R² for each individual compound corresponds in each case to one line A-1 to A-323 of Table A (compounds I.E.A-1 to I.E.A-323).

Table 6: Compounds of the formula I.F, in which R¹ and R² for each individual compound corresponds in each case to one line A-1 to A-323 of Table A (compounds I.F.A-1 to I.F.A-323).

Table 7: Compounds of the formula I.G, in which R¹ and R² for each individual compound corresponds in each case to one line A-1 to A-323 of Table A(compounds I.G.A-1 to I.G.A-323).

Table 8: Compounds of the formula I.H, in which R¹ and R² for each individual compound corresponds in each case to one line A-1 to A-323 of Table A (compounds I.H.A-1 to I.H.A-323).

Table 9: Compounds of the formula I.J, in which R¹ and R² for each individual compound corresponds in each case to one line A-1 to A-323 of Table A (compounds I.J.A-1 to I.J.A-323).

Table 10: Compounds of the formula I.K, in which R¹ and R² for each individual compound corresponds in each case to one line A-1 to A-323 of Table A (compounds I.K.A-1 to I.K.A-323).

Table 11: Compounds of the formula I.L, in which R¹ and R² for each individual compound corresponds in each case to one line A-1 to A-323 of Table A (compounds I.L.A-1 to I.L.A-323).

TABLE A

| No | R¹ | R² |
|---|---|---|
| A-1 | difluoromethyl | hydrogen |
| A-2 | trifluoromethyl | hydrogen |
| A-3 | 2,2-difluoroethyl | hydrogen |
| A-4 | 2,2,2-trifluoroethyl | hydrogen |
| A-5 | 2-chloro-2- | hydrogen |
| A-6 | 2-chloro-2,2- | hydrogen |
| A-7 | 2,2,2-trichloroethyl | hydrogen |
| A-8 | pentafluoroethyl | hydrogen |
| A-9 | 3,3,3-trifluoropropyl | hydrogen |
| A-10 | CH₂CF₂CF₃ | hydrogen |
| A-11 | CH₂CF₂CF₃ | hydrogen |
| A-12 | CH(CH₃)CF₃ | hydrogen |
| A-13 | CH₂CF₂CF₃ | hydrogen |
| A-14 | CH₂C(CH₃)₂F | hydrogen |
| A-15 | CH₂CH(CH₃)CF₃ | hydrogen |
| A-16 | CH₂C(CH₃)₂CF₃ | hydrogen |
| A-17 | 2,2-difluoro-cycloproplymethyl | hydrogen |
| A-18 | difluoromethyl | methyl |
| A-19 | trifluoromethyl | methyl |
| A-20 | 2,2-difluoroethyl | methyl |
| A-21 | 2,2,2-trifluoroethyl | methyl |
| A-22 | 2-chloro-2- | methyl |
| A-23 | 2-chloro-2,2- | methyl |
| A-24 | 2,2,2-trichloroethyl | methyl |
| A-25 | pentafluoroethyl | methyl |
| A-26 | 3,3,3-trifluoropropyl | methyl |
| A-27 | CH₂CF₂CF₃ | methyl |
| A-28 | CH₂CF₂CF₃ | methyl |
| A-29 | CH(CH₃)CF₃ | methyl |
| A-30 | CH₂CF₂CH₃ | methyl |
| A-31 | CH₂C(CH₃)₂F | methyl |
| A-32 | CH₂CH(CH₃)CF₃ | methyl |
| A-33 | CH₂C(CH₃)₂CF₃ | methyl |
| A-34 | 2,2-difluoro-cycloproplymethyl | methyl |
| A-35 | difluoromethyl | ethyl |
| A-36 | trifluoromethyl | ethyl |
| A-37 | 2,2-difluoroethyl | ethyl |
| A-38 | 2,2,2-trifluoroethyl | ethyl |
| A-39 | 2-chloro-2- | ethyl |
| A-40 | 2-chloro-2,2- | ethyl |
| A-41 | 2,2,2-trichloroethyl | ethyl |
| A-42 | pentafluoroethyl | ethyl |
| A-43 | 3,3,3-trifluoropropyl | ethyl |
| A-44 | CH₂CF₂CF₃ | ethyl |
| A-45 | CF₂CF₂CF₃ | ethyl |
| A-46 | CH(CH₃)CF₃ | ethyl |
| A-47 | CH₂CF₂CH₃ | ethyl |
| A-48 | CH₂C(CH₃)₂F | ethyl |
| A-49 | CH₂CH(CH₃)CF₃ | ethyl |
| A-50 | CH₂C(CH₃)₂CF₃ | ethyl |
| A-51 | 2,2-difluoro-cycloproplymethyl | ethyl |
| A-52 | difluoromethyl | n-propyl |
| A-53 | trifluoromethyl | n-propyl |
| A-54 | 2,2-difluoroethyl | n-propyl |
| A-55 | 2,2,2-trifluoroethyl | n-propyl |
| A-56 | 2-chloro-2- | n-propyl |
| A-57 | 2-chloro-2,2- | n-propyl |
| A-58 | 2,2,2-trichloroethyl | n-propyl |
| A-59 | pentafluoroethyl | n-propyl |
| A-60 | 3,3,3-trifluoropropyl | n-propyl |
| A-61 | CH₂CF₂CF₃ | n-propyl |
| A-62 | CF₂CF₂CF₃ | n-propyl |
| A-63 | CH(CH₃)CF₃ | n-propyl |
| A-64 | CH₂CF₂CH₃ | n-propyl |
| A-65 | CH₂C(CH₃)₂F | n-propyl |
| A-66 | CH₂CH(CH₃)CF₃ | n-propyl |
| A-67 | CH₂C(CH₃)₂CF₃ | n-propyl |
| A-68 | 2,2-difluoro-cycloproplymethyl | n-propyl |
| A-69 | difluoromethyl | vinyl |
| A-70 | trifluoromethyl | vinyl |
| A-71 | 2,2-difluoroethyl | vinyl |
| A-72 | 2,2,2-trifluoroethyl | vinyl |
| A-73 | 2-chloro-2- | vinyl |
| A-74 | 2-chloro-2,2- | vinyl |
| A-75 | 2,2,2-trichloroethyl | vinyl |
| A-76 | pentafluoroethyl | vinyl |
| A-77 | 3,3,3-trifluoropropyl | vinyl |
| A-78 | CH₂CF₂CF₃ | vinyl |
| A-79 | CF₂CF₂CF₃ | vinyl |
| A-80 | CH(CH₃)CF₃ | vinyl |
| A-81 | CH₂CF₂CH₃ | vinyl |
| A-82 | CH₂C(CH₃)₂F | vinyl |
| A-83 | CH₂CH(CH₃)CF₃ | vinyl |
| A-84 | CH₂C(CH₃)₂CF₃ | vinyl |
| A-85 | 2,2-difluoro-cycloproplymethyl | vinyl |
| A-86 | difluoromethyl | tert-butyl |
| A-87 | trifluoromethyl | tert-butyl |
| A-88 | 2,2-difluoroethyl | tert-butyl |
| A-89 | 2,2,2-trifluoroethyl | tert-butyl |
| A-90 | 2-chloro-2- | tert-butyl |
| A-91 | 2-chloro-2,2- | tert-butyl |
| A-92 | 2,2,2-trichloroethyl | tert-butyl |
| A-93 | pentafluoroethyl | tert-butyl |
| A-94 | 3,3,3-trifluoropropyl | tert-butyl |
| A-95 | CH₂CF₂CF₃ | tert-butyl |
| A-96 | CF₂CF₂CF₃ | tert-butyl |
| A-97 | CH(CH₃)CF₃ | tert-butyl |
| A-98 | CH₂CF₂CH₃ | tert-butyl |
| A-99 | CH₂C(CH₃)₂F | tert-butyl |
| A-100 | CH₂CH(CH₃)CF₃ | tert-butyl |
| A-101 | CH₂C(CH₃)₂CF₃ | tert-butyl |
| A-102 | 2,2-difluoro-cycloproplymethyl | tert-butyl |
| A-103 | difluoromethyl | 1-methyl-propyl |
| A-104 | trifluoromethyl | 1-methyl-propyl |
| A-105 | 2,2-difluoroethyl | 1-methyl-propyl |
| A-106 | 2,2,2-trifluoroethyl | 1-methyl-propyl |
| A-107 | 2-chloro-2-fluoroethyl | 1-methyl-propyl |
| A-108 | 2-chloro-2,2-difluoroethyl | 1-methyl-propyl |
| A-109 | 2,2,2-trichloroethyl | 1-methyl-propyl |
| A-110 | pentafluoroethyl | 1-methyl-propyl |
| A-111 | 3,3,3-trifluoropropyl | 1-methyl-propyl |
| A-112 | CH₂CF₂CF₃ | 1-methyl-propyl |
| A-113 | CF₂CF₂CF₃ | 1-methyl-propyl |
| A-114 | CH(CH₃)CF₃ | 1-methyl-propyl |
| A-115 | CH₂CF₂CH₃ | 1-methyl-propyl |
| A-116 | CH₂C(CH₃)₂F | 1-methyl-propyl |
| A-117 | CH₂CH(CH₃)CF₃ | 1-methyl-propyl |
| A-118 | CH₂C(CH₃)₂CF₃ | 1-methyl-propyl |
| A-119 | 2,2-difluoro-cycloproplymethyl | 1-methyl-propyl |
| A-120 | difluoromethyl | iso-propyl |
| A-121 | trifluoromethyl | iso-propyl |
| A-122 | 2,2-difluoroethyl | iso-propyl |
| A-123 | 2,2,2-trifluoroethyl | iso-propyl |
| A-124 | 2-chloro-2- | iso-propyl |
| A-125 | 2-chloro-2,2- | iso-propyl |
| A-126 | 2,2,2-trichloroethyl | iso-propyl |
| A-127 | pentafluoroethyl | iso-propyl |
| A-128 | 3,3,3-trifluoropropyl | iso-propyl |
| A-129 | CH₂CF₂CF₃ | iso-propyl |
| A-130 | CF₂CF₂CF₃ | iso-propyl |
| A-131 | CH(CH₃)CF₃ | iso-propyl |
| A-132 | CH₂CF₂CH₃ | iso-propyl |
| A-133 | CH₂C(CH₃)₂F | iso-propyl |
| A-134 | CH₂CH(CH₃)CF₃ | iso-propyl |
| A-135 | CH₂C(CH₃)₂CF₃ | iso-propyl |
| A-136 | 2,2-difluoro-cycloproplymethyl | iso-propyl |
| A-137 | difluoromethyl | cyclopropyl |
| A-138 | trifluoromethyl | cyclopropyl |
| A-139 | 2,2-difluoroethyl | cyclopropyl |
| A-140 | 2,2,2-trifluoroethyl | cyclopropyl |
| A-141 | 2-chloro-2- | cyclopropyl |
| A-142 | 2-chloro-2,2- | cyclopropyl |
| A-143 | 2,2,2-trichloroethyl | cyclopropyl |
| A-144 | pentafluoroethyl | cyclopropyl |
| A-145 | 3,3,3-trifluoropropyl | cyclopropyl |
| A-146 | CH₂CF₂CF₃ | cyclopropyl |
| A-147 | CF₂CF₂CF₃ | cyclopropyl |
| A-148 | CH(CH₃)CF₃ | cyclopropyl |
| A-149 | CH₂CF₂CH₃ | cyclopropyl |
| A-150 | CH₂C(CH₃)₂F | cyclopropyl |
| A-151 | CH₂CH(CH₃)CF₃ | cyclopropyl |
| A-152 | CH₂C(CH₃)₂CF₃ | cyclopropyl |
| A-153 | 2,2-difluoro-cycloproplymethyl | cyclopropyl |
| A-154 | difluoromethyl | cyclobutyl |
| A-155 | trifluoromethyl | cyclobutyl |
| A-156 | 2,2-difluoroethyl | cyclobutyl |
| A-157 | 2,2,2-trifluoroethyl | cyclobutyl |
| A-158 | 2-chloro-2- | cyclobutyl |
| A-159 | 2-chloro-2,2- | cyclobutyl |
| A-160 | 2,2,2-trichloroethyl | cyclobutyl |
| A-161 | pentafluoroethyl | cyclobutyl |
| A-162 | 3,3,3-trifluoropropyl | cyclobutyl |
| A-163 | CH₂CF₂CF₃ | cyclobutyl |
| A-164 | CF₂CF₂CF₃ | cyclobutyl |
| A-165 | CH(CH₃)CF₃ | cyclobutyl |
| A-166 | CH₂CF₂CH₃ | cyclobutyl |
| A-167 | CH₂C(CH₃)₂F | cyclobutyl |
| A-168 | CH₂CH(CH₃)CF₃ | cyclobutyl |
| A-169 | CH₂C(CH₃)₂CF₃ | cyclobutyl |
| A-170 | 2,2-difluoro-cycloproplymethyl | cyclobutyl |
| A-171 | difluoromethyl | cyclopentyl |
| A-172 | trifluoromethyl | cyclopentyl |
| A-173 | 2,2-difluoroethyl | cyclopentyl |
| A-174 | 2,2,2-trifluoroethyl | cyclopentyl |
| A-175 | 2-chloro-2- | cyclopentyl |
| A-176 | 2-chloro-2,2- | cyclopentyl |
| A-177 | 2,2,2-trichloroethyl | cyclopentyl |
| A-178 | pentafluoroethyl | cyclopentyl |
| A-179 | 3,3,3-trifluoropropyl | cyclopentyl |
| A-180 | CH₂CF₂CF₃ | cyclopentyl |
| A-181 | CF₂CF₂CF₃ | cyclopentyl |
| A-182 | CH(CH₃)CF₃ | cyclopentyl |
| A-183 | CH₂CF₂CH₃ | cyclopentyl |
| A-184 | CH₂C(CH₃)₂F | cyclopentyl |
| A-185 | CH₂CH(CH₃)CF₃ | cyclopentyl |
| A-186 | CH₂C(CH₃)₂CF₃ | cyclopentyl |
| A-187 | 2,2-difluoro-cycloproplymethyl | cyclopentyl |
| A-188 | difluoromethyl | cyclohexyl |
| A-189 | trifluoromethyl | cyclohexyl |
| A-190 | 2,2-difluoroethyl | cyclohexyl |
| A-191 | 2,2,2-trifluoroethyl | cyclohexyl |
| A-192 | 2-chloro-2- | cyclohexyl |
| A-193 | 2-chloro-2,2- | cyclohexyl |
| A-194 | 2,2,2-trichloroethyl | cyclohexyl |
| A-195 | pentafluoroethyl | cyclohexyl |
| A-196 | 3,3,3-trifluoropropyl | cyclohexyl |
| A-197 | CH₂CF₂CF₃ | cyclohexyl |
| A-198 | CF₂CF₂CF₃ | cyclohexyl |
| A-199 | CH(CH₃)CF₃ | cyclohexyl |
| A-200 | CH₂CF₂CH₃ | cyclohexyl |
| A-201 | CH₂C(CH₃)₂F | cyclohexyl |
| A-202 | CH₂CH(CH₃)CF₃ | cyclohexyl |
| A-203 | CH₂C(CH₃)₂CF₃ | cyclohexyl |
| A-204 | 2,2-difluoro-cycloproplymethyl | cyclohexyl |
| A-205 | difluoromethyl | allyl |
| A-206 | trifluoromethyl | allyl |
| A-207 | 2,2-difluoroethyl | allyl |
| A-208 | 2,2,2-trifluoroethyl | allyl |
| A-209 | 2-chloro-2- | allyl |
| A-210 | 2-chloro-2,2- | allyl |
| A-211 | 2,2,2-trichloroethyl | allyl |
| A-212 | pentafluoroethyl | allyl |
| A-213 | 3,3,3-trifluoropropyl | allyl |
| A-214 | CH₂CF₂CF₃ | allyl |
| A-215 | CF₂CF₂CF₃ | allyl |
| A-216 | CH(CH₃)CF₃ | allyl |
| A-217 | CH₂CF₂CH₃ | allyl |
| A-218 | CH₂C(CH₃)₂F | allyl |
| A-219 | CH₂CH(CH₃)CF₃ | allyl |
| A-220 | CH₂C(CH₃)₂CF₃ | allyl |
| A-221 | 2,2-difluoro-cycloproplymethyl | allyl |
| A-222 | difluoromethyl | phenyl |
| A-223 | trifluoromethyl | phenyl |
| A-224 | 2,2-difluoroethyl | phenyl |
| A-225 | 2,2,2-trifluoroethyl | phenyl |
| A-226 | 2-chloro-2- | phenyl |
| A-227 | 2-chloro-2,2- | phenyl |
| A-228 | 2,2,2-trichloroethyl | phenyl |
| A-229 | pentafluoroethyl | phenyl |
| A-230 | 3,3,3-trifluoropropyl | phenyl |
| A-231 | CH₂CF₂CF₃ | phenyl |
| A-232 | CF₂CF₂CF₃ | phenyl |
| A-233 | CH(CH₃)CF₃ | phenyl |
| A-234 | CH₂CF₂CH₃ | phenyl |
| A-235 | CH₂C(CH₃)₂F | phenyl |
| A-236 | CH₂CH(CH₃)CF₃ | phenyl |
| A-237 | CH₂C(CH₃)₂CF₃ | phenyl |
| A-238 | 2,2-difluoro-cycloproplymethyl | phenyl |
| A-239 | difluoromethyl | 4-fluoro-phenyl |
| A-240 | trifluoromethyl | 4-fluoro-phenyl |
| A-241 | 2,2-difluoroethyl | 4-fluoro-phenyl |
| A-242 | 2,2,2-trifluoroethyl | 4-fluoro-phenyl |
| A-243 | 2-chloro-2-fluoroethyl | 4-fluoro-phenyl |
| A-244 | 2-chloro-2,2-difluoroethyl | 4-fluoro-phenyl |
| A-245 | 2,2,2-trichloroethyl | 4-fluoro-phenyl |
| A-246 | pentafluoroethyl | 4-fluoro-phenyl |
| A-247 | 3,3,3-trifluoropropyl | 4-fluoro-phenyl |
| A-248 | CH₂CF₂CF₃ | 4-fluoro-phenyl |
| A-249 | CF₂CF₂CF₃ | 4-fluoro-phenyl |
| A-250 | CH(CH₃)CF₃ | 4-fluoro-phenyl |
| A-251 | CH₂CF₂CH₃ | 4-fluoro-phenyl |
| A-252 | CH₂C(CH₃)₂F | 4-fluoro-phenyl |
| A-253 | CH₂CH(CH₃)CF₃ | 4-fluoro-phenyl |
| A-254 | CH₂C(CH₃)₂CF₃ | 4-fluoro-phenyl |
| A-255 | 2,2-difluoro-cycloproplymethyl | 4-fluoro-phenyl |
| A-256 | difluoromethyl | 2-fluoro-phenyl |
| A-257 | trifluoromethyl | 2-fluoro-phenyl |
| A-258 | 2,2-difluoroethyl | 2-fluoro-phenyl |
| A-259 | 2,2,2-trifluoroethyl | 2-fluoro-phenyl |
| A-260 | 2-chloro-2-fluoroethyl | 2-fluoro-phenyl |
| A-261 | 2-chloro-2,2-difluoroethyl | 2-fluoro-phenyl |
| A-262 | 2,2,2-trichloroethyl | 2-fluoro-phenyl |
| A-263 | pentafluoroethyl | 2-fluoro-phenyl |
| A-264 | 3,3,3-trifluoropropyl | 2-fluoro-phenyl |
| A-265 | CH₂CF₂CF₃ | 2-fluoro-phenyl |
| A-266 | CF₂CF₂CF₃ | 2-fluoro-phenyl |
| A-267 | CH(CH₃)CF₃ | 2-fluoro-phenyl |
| A-268 | CH₂CF₂CH₃ | 2-fluoro-phenyl |
| A-269 | CH₂C(CH₃)₂F | 2-fluoro-phenyl |
| A-270 | CH₂CH(CH₃)CF₃ | 2-fluoro-phenyl |
| A-271 | CH₂C(CH₃)₂CF₃ | 2-fluoro-phenyl |
| A-272 | 2,2-difluoro-cycloproplymethyl | 2-fluoro-phenyl |
| A-273 | difluoromethyl | 2,4-difluoro-phenyl |
| A-274 | trifluoromethyl | 2,4-difluoro-phenyl |
| A-275 | 2,2-difluoroethyl | 2,4-difluoro-phenyl |
| A-276 | 2,2,2-trifluoroethyl | 2,4-difluoro-phenyl |
| A-277 | 2-chloro-2-fluoroethyl | 2,4-difluoro-phenyl |
| A-278 | 2-chloro-2,2-difluoroethyl | 2,4-difluoro-phenyl |
| A-279 | 2,2,2-trichloroethyl | 2,4-difluoro-phenyl |
| A-280 | pentafluoroethyl | 2,4-difluoro-phenyl |
| A-281 | 3,3,3-trifluoropropyl | 2,4-difluoro-phenyl |
| A-282 | CH₂CF₂CF₃ | 2,4-difluoro-phenyl |
| A-283 | CF₂CF₂CF₃ | 2,4-difluoro-phenyl |
| A-284 | CH(CH₃)CF₃ | 2,4-difluoro-phenyl |
| A-285 | CH₂CF₂CH₃ | 2,4-difluoro-phenyl |
| A-286 | CH₂C(CH₃)₂F | 2,4-difluoro-phenyl |
| A-287 | CH₂CH(CH₃)CF₃ | 2,4-difluoro-phenyl |
| A-288 | CH₂C(CH₃)₂CF₃ | 2,4-difluoro-phenyl |
| A-289 | 2,2-difluoro-cycloproplymethyl | 2,4-difluoro-phenyl |
| A-290 | difluoromethyl | —NH(CH₃) |
| A-291 | trifluoromethyl | —NH(CH₃) |
| A-292 | 2,2-difluoroethyl | —NH(CH₃) |
| A-293 | 2,2,2-trifluoroethyl | —NH(CH₃) |
| A-294 | 2-chloro-2-fluoroethyl | —NH(CH₃) |
| A-295 | 2-chloro-2,2-difluoroethyl | —NH(CH₃) |
| A-296 | 2,2,2-trichloroethyl | —NH(CH₃) |
| A-297 | pentafluoroethyl | —NH(CH₃) |
| A-298 | 3,3,3-trifluoropropyl | —NH(CH₃) |
| A-299 | CH₂CF₂CF₃ | —NH(CH₃) |
| A-300 | CF₂CF₂CF₃ | —NH(CH₃) |
| A-301 | CH(CH₃)CF₃ | —NH(CH₃) |
| A-302 | CH₂CF₂CH₃ | —NH(CH₃) |
| A-303 | CH₂C(CH₃)₂F | —NH(CH₃) |
| A-304 | CH₂CH(CH₃)CF₃ | —NH(CH₃) |
| A-305 | CH₂C(CH₃)₂CF₃ | —NH(CH₃) |
| A-306 | 2,2-difluoro-cycloproplymethyl | —NH(CH₃) |
| A-307 | difluoromethyl | —N(CH₃)₂ |
| A-308 | trifluoromethyl | —N(CH₃)₂ |
| A-309 | 2,2-difluoroethyl | —N(CH₃)₂ |
| A-310 | 2,2,2-trifluoroethyl | —N(CH₃)₂ |
| A-311 | 2-chloro-2-fluoroethyl | —N(CH₃)₂ |
| A-312 | 2-chloro-2,2-difluoroethyl | —N(CH₃)₂ |
| A-313 | 2,2,2-trichloroethyl | —N(CH₃)₂ |
| A-314 | pentafluoroethyl | —N(CH₃)₂ |
| A-315 | 3,3,3-trifluoropropyl | —N(CH₃)₂ |
| A-316 | CH₂CF₂CF₃ | —N(CH₃)₂ |
| A-317 | CF₂CF₂CF₃ | —N(CH₃)₂ |
| A-318 | CH(CH₃)CF₃ | —N(CH₃)₂ |
| A-319 | CH₂CF₂CH₃ | —N(CH₃)₂ |
| A-320 | CH₂C(CH₃)₂F | —N(CH₃)₂ |
| A-321 | CH₂CH(CH₃)CF₃ | —N(CH₃)₂ |
| A-322 | CH₂C(CH₃)₂CF₃ | —N(CH₃)₂ |
| A-323 | 2,2-difluoro-cycloproplymethyl | —N(CH₃)₂ |

It is understood that when in aqueous media, the compounds of formula I according to the invention ma be present in a reversible equilibrium with the corresponding covalently hydrated forms (i.e., the compounds of formula Ia and formula Ib as shown below) at the CF₃-oxadiazole motif. This dynamic equilibrium may be important for the biological activity of the compounds of formula I. The designations of R^{A}, n, L, R¹, R², R³ and R⁴ with reference to the compounds of formula I of the present invention apply generally to the compounds of formula Ia and Ib, as do the specific disclosures of combinations of R¹ and R² as represented for the individual compounds disclosed in Tables 1 to 11 or the individual compounds disclosed in Table I. The variable “A” in the formulae depicted hereinafter shall represent the phenyl group in compounds of the formula I, which may be unsubstituted or substituted with 1 or 2 radicals R^{A}.

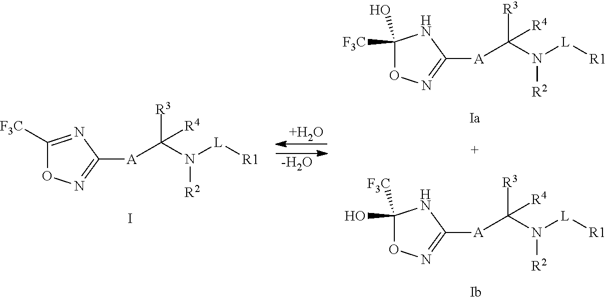

The compounds of the formula I can be prepared according to methods or in analogy to methods that are described in the prior art. The synthesis takes advantage of starting materials that are commercially available or may be prepared according to conventional procedures starting from readily available compounds. For example, compounds of the formula I can be prepared by reacting of oxadiazole amine II with compounds of type III in an organic solvent and in the presence of a base, wherein L is —S(═O)ₚ— or wherein, for compounds I.A, I.B, I.C, I.D, I.E, L is —C(═O)— and wherein LG is, for example, chloride or LG together with the group -L-R¹ forms an anhydride. Alternatively compound I can be obtained by reacting of compound II with the corresponding acid III (LG=OH) using peptide coupling reaction conditions such as EDCI and HOBt (for precedents see for example Bioorganic & Medicinal Chemistry Letters, 20(15), 4550-4554; 2010)

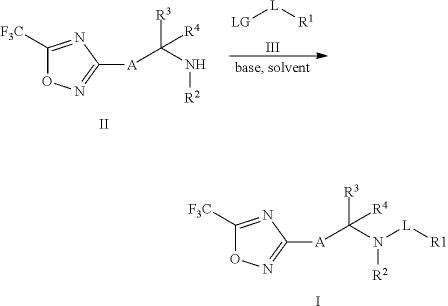

Compounds of the formula II can be prepared by reacting amidines of type IV with trifluoroacetic anhydride in an organic solvent, preferably an ethereal solvent at temperatures between 0° C. and 100° C., preferably at room temperature, as previously described in WO2013/008162 or in Kitamura et al. Chem. Pharm. Bull. 2001, 49, 268.

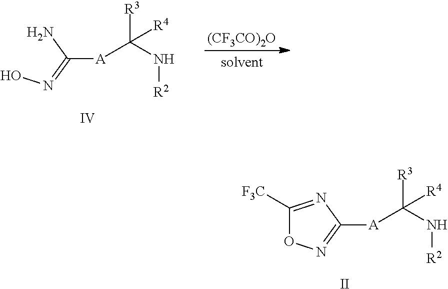

A skilled person will recognize that compounds of type IV can be accessed by treating nitriles of type V with hydroxylamine (or its HCl salt) in an organic solvent and in the presence of a base (for precedents see for example WO2009/074950, WO2006/013104, EP1932843 or in Kitamura et al. Chem. Pharm. Bull. 2001, 49, 268).

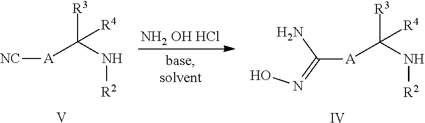

Compounds V are either commercially available or can be accessed through known methods that are known to a person skilled in the art.

Alternatively compounds I can be prepared from compounds of type VI by treatment with trifluoroacetic anhydride in a suitable solvent, such as tetrahydrofuran, at a temperature between 0° C. and 25° C. For related examples, see Kitamura, S et al. Chem. Pharm. Bull, 2001, 49, 268.

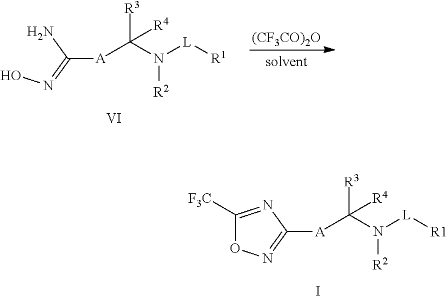

In one aspect the invention relates to a process for preparing compounds of the formula I, which comprises the process step of reacting a compound of the formula VI with trifluoroacetic anhydride, to give compounds of the formula I, and wherein the variables R^{A}, n, L, R¹, R², R³ and R⁴ are as defined or preferably defined herein for compounds of the formula I. Another embodiment of the invention relates to intermediate compounds of the formula VI, wherein the variables R^{A}, n, L, R¹, R², R³ and R⁴ are as defined or preferably defined herein for compounds of the formula I.

Preferably the group L in compounds of the formula VI is —S(═O)₂—.

In one aspect the invention relates to intermediate compounds of the formula VI.a

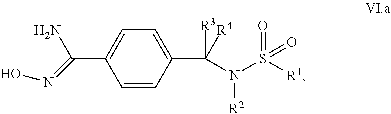

wherein the variables R¹, R², R³ and R⁴ are as defined or preferably defined herein for compounds of the formula I.

In a further embodiment the invention relates to intermediate compounds of the formula VI.a, wherein R³ and R⁴ independently of each other are hydrogen, halogen, C₁-C₆-alkyl or C₁-C₆-haloalkyl; or R³ and R⁴ together with the carbon atom to which they are bound form a cyclopropyl ring; R² is hydrogen, C₁-C₆-alkyl, C₂-C₆-alkenyl, C₂-C₆-alkynyl, C₃-C₈-cycloalkyl or C₃-C₈-cycloalkyl-C₁-C₄-alkyl; and wherein R¹ is C₁-C₆-alkyl; and wherein the alkyl group is substituted with 1, 2, 3, 4, 5 or up to the maximum possible number of identical or different halogen atoms; preferably the halogen atom is fluorine; and wherein the alkyl group is further unsubstituted or, in addition to the halogen atoms, substituted with 1, 2, 3 or up to the maximum possible number of identical or different groups selected from the group consisting of C₁-C₆-alkyl or C₃-C₈-cycloalkyl; and wherein the cycloalkyl group is unsubstituted or substituted with 1, 2, 3 or 4 or up to the maximum possible number of radicals selected from the group consisting of chlorine and fluorine.

In a further embodiment the invention relates to intermediate compounds of the formula VI.a, wherein R³ and R⁴ independently of each other are hydrogen, halogen, C₁-C₆-alkyl or C₁-C₆-haloalkyl; or R³ and R⁴ together with the carbon atom to which they are bound form a cyclopropyl ring; in particular R³ and R⁴ are hydrogen; and R² is C₁-C₆-alkyl, C₂-C₆-alkenyl, C₂-C₆-alkynyl, C₃-C₈-cycloalkyl or C₃-C₈-cycloalkyl-C₁-C₄-alkyl; and wherein R¹ is selected from the group consisting of difluoromethyl, trifluoromethyl, 2,2-difluoroethyl, 2,2,2-trifluoroethyl, 2-chloro-2-fluoroethyl, 2-chloro-2,2-difluoroethyl, 2,2,2-trichloroethyl and pentafluoroethyl, 3,3,3-trifluoropropyl, CH₂CF₂CF₃ or CF₂CF₂CF₅, CH(CH₃)CF₃, CH₂CF₂CH₃, CH₂C(CH₃)₂F, CH₂CH(CH₃)CF₃, CH₂C(CH₃)₂CF₃ and 2,2-difluorocycloproplymethyl.

Especially preferred intermediates are compounds of the formula VI.a, wherein R³ and R⁴ are hydrogen; R² is hydrogen, methyl, ethyl, iso-propyl, cyclopropyl or allyl; and wherein R¹ is 2,2,2-trifluoroethyl or 2,2-difluorocycloproplymethyl.

Particularly preferred examples of intermediates are compounds of the formula VI.a, namely compounds VI.a.A-1 to VI.a.A-323, wherein the meaning of the radicals R¹ and R² for each individual compound corresponds in each case to one line A-1 to A-323 of Table A; and wherein the meaning of the radicals R³ and R⁴ is hydrogen.

Compounds of the formula VI can be prepared from compounds of the formula VII by treating them with a hydroxylamine hydrochloride salt in the presence of a base, such as sodium carbonate, in a suitable solvent, such as methanol, at a temperature between 0° C. and 100° C. For related examples, see Kitamura, S et al. Chem. Pharm. Bull, 2001, 49, 268.

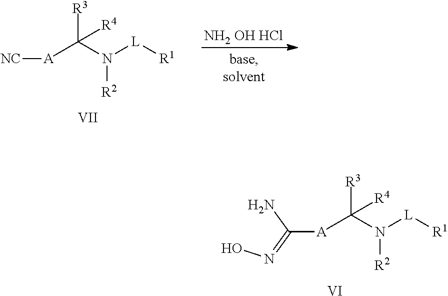

Accordingly, another embodiment of the invention relates to intermediate compounds of the formula VII, wherein the variables R^{A}, n, L, R¹, R², R³ and R⁴ are as defined or preferably defined herein for compounds of the formula I.

Preferably the group L in compounds of the formula VII is —S(═O)₂—. Preferably A is phenyl. In one aspect the invention relates to intermediate compounds of the formula VII.a

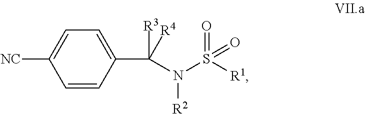

wherein the variables R¹, R², R³ and R⁴ are as defined or preferably defined herein for compounds of the formula I.

In a further embodiment the invention relates to intermediate compounds of the formula VII.a, wherein R³ and R⁴ independently of each other are hydrogen, halogen, C₁-C₆-alkyl or C₁-C₆-haloalkyl; or R³ and R⁴ together with the carbon atom to which they are bound form a cyclopropyl ring; R² is hydrogen, C₁-C₆-alkyl, C₂-C₆-alkenyl, C₂-C₆-alkynyl, C₃-C₈-cycloalkyl or C₃-C₈-cycloalkyl-C₁-C₄-alkyl; and wherein R¹ is C₁-C₆-alkyl; and wherein the alkyl group is substituted with 1, 2, 3, 4, 5 or up to the maximum possible number of identical or different halogen atoms; preferably the halogen atom is fluorine; and wherein the alkyl group is further unsubstituted or, in addition to the halogen atoms, substituted with 1, 2, 3 or up to the maximum possible number of identical or different groups selected from the group consisting of C₁-C₆-alkyl or C₃-C₈-cycloalkyl; and wherein the cycloalkyl group is unsubstituted or substituted with 1, 2, 3 or 4 or up to the maximum possible number of radicals selected from the group consisting of chlorine and fluorine.

In a further embodiment the invention relates to intermediate compounds of the formula VII.a, wherein R³ and R⁴ independently of each other are hydrogen, halogen, C₁-C₆-alkyl or C₁-C₆-haloalkyl; or R³ and R⁴ together with the carbon atom to which they are bound form a cyclopropyl ring; in particular R³ and R⁴ are hydrogen; and R² is C₁-C₆-alkyl, C₂-C₆-alkenyl, C₂-C₆-alkynyl, C₃-C₈-cycloalkyl or C₃-C₈-cycloalkyl-C₁-C₄-alkyl; and wherein R¹ is selected from the group consisting of difluoromethyl, trifluoromethyl, 2,2-difluoroethyl, 2,2,2-trifluoroethyl, 2-chloro-2-fluoroethyl, 2-chloro-2,2-difluoroethyl, 2,2,2-trichloroethyl and pentafluoroethyl, 3,3,3-trifluoropropyl, CH₂CF₂CF₃ or CF₂CF₂CF₅, CH(CH₃)CF₃, CH₂CF₂CH₃, CH₂C(CH₃)₂F, CH₂CH(CH₃)CF₃, CH₂C(CH₃)₂CF₃ and 2,2-difluorocycloproplymethyl.

Especially preferred intermediates are compounds of the formula VII.a, wherein R³ and R⁴ are hydrogen; R² is hydrogen, methyl, ethyl, iso-propyl, cyclopropyl or allyl; and wherein R¹ is 2,2,2-trifluoroethyl or 2,2-difluorocycloproplymethyl.

Particularly preferred examples of intermediates are compounds of the formula VII.a, namely compounds VII.a.A-1 to VII.a.A-323, wherein the meaning of the radicals R¹ and R² for each individual compound corresponds in each case to one line A-1 to A-323 of Table A; and wherein the meaning of the radicals R³ and R⁴ is hydrogen.

The compounds of the formula VII can be prepared by reacting of amine V with compounds of type III in analogy to the procedure described above for the transformation of compounds of the formula II to compounds of the formula I.

The compounds I and the compositions according to the invention are particularly important in the control of a multitude of phytopathogenic fungi on various cultivated plants, such as cereals, e. g. wheat, rye, barley, triticale, oats or rice; beet, e. g. sugar beet or fodder beet; fruits, such as pomes, stone fruits or soft fruits, e. g. apples, pears, plums, peaches, almonds, cherries, strawberries, raspberries, blackberries or gooseberries; leguminous plants, such as lentils, peas, alfalfa or soybeans; oil plants, such as rape, mustard, olives, sunflowers, coconut, cocoa beans, castor oil plants, oil palms, ground nuts or soybeans; cucurbits, such as squashes, cucumber or melons; fiber plants, such as cotton, flax, hemp or jute; citrus fruit, such as oranges, lemons, grapefruits or mandarins; vegetables, such as spinach, lettuce, asparagus, cabbages, carrots, onions, tomatoes, potatoes, cucurbits or paprika; lauraceous plants, such as avocados, cinnamon or camphor; energy and raw material plants, such as corn, soybean, rape, sugar cane or oil palm; corn; tobacco; nuts; coffee; tea; bananas; vines (table grapes and grape juice grape vines); hop; turf; sweet leaf (also called Stevia); natural rubber plants or ornamental and forestry plants, such as flowers, shrubs, broad-leaved trees or evergreens, e. g. conifers; and on the plant propagation material, such as seeds, and the crop material of these plants. Preferably, compounds I and compositions thereof, respectively are used for controlling a multitude of fungi on field crops, such as potatoes sugar beets, tobacco, wheat, rye, barley, oats, rice, corn, cotton, soybeans, rape, legumes, sunflowers, coffee or sugar cane; fruits; vines; ornamentals; or vegetables, such as cucumbers, tomatoes, beans or squashes.

The term “plant propagation material” is to be understood to denote all the generative parts of the plant such as seeds and vegetative plant material such as cuttings and tubers (e. g. potatoes), which can be used for the multiplication of the plant. This includes seeds, roots, fruits, tubers, bulbs, rhizomes, shoots, sprouts and other parts of plants, including seedlings and young plants, which are to be transplanted after germination or after emergence from soil. These young plants may also be protected before transplantation by a total or partial treatment by immersion or pouring.

Preferably, treatment of plant propagation materials with compounds I and compositions thereof, respectively, is used for controlling a multitude of fungi on cereals, such as wheat, rye, barley and oats; rice, corn, cotton and soybeans.

The term “cultivated plants” is to be understood as including plants which have been modified by mutagenesis or genetic engineering to provide a new trait to a plant or to modify an already present trait. Mutagenesis includes techniques of random mutagenesis using X-rays or mutagenic chemicals, but also techniques of targeted mutagenesis, to create mutations at a specific locus of a plant genome. Targeted mutagenesis techniques frequently use oligonucleotides or proteins like CRISPR/Cas, zinc-finger nucleases, TALENs or mega-nucleases to achieve the targeting effect. Genetic engineering usually uses recombinant DNA techniques to create modifications in a plant genome which under natural circumstances cannot readily be obtained by cross breeding, mutagenesis or natural recombination. Typically, one or more genes are integrated into the genome of a plant to add a trait or improve a trait. These integrated genes are also referred to as transgenes in the art, while plant comprising such transgenes are referred to as transgenic plants. The process of plant transformation usually produces several transformation events, which differ in the genomic locus in which a transgene has been integrated. Plants comprising a specific transgene on a specific genomic locus are usually described as comprising a specific “event”, which is referred to by a specific event name. Traits which have been introduced in plants or have been modified include herbicide tolerance, insect resistance, increased yield and tolerance to abiotic conditions, like drought. Herbicide tolerance has been created by using mutagenesis as well as using genetic engineering. Plants which have been rendered tolerant to acetolactate synthase (ALS) inhibitor herbicides by mutagenesis and breeding comprise plant varieties commercially available under the name Clearfield®.

Herbicide tolerance has been created via the use of transgenes to glyphosate, glufosinate, 2,4-D, dicamba, oxynil herbicides, like bromoxynil and ioxynil, sulfonylurea herbicides, ALS inhibitors and 4-hydroxyphenylpyruvate dioxygenase (HPPD) inhibitors, like isoxaflutole and mesotrione.

Transgenes which have been used to provide herbicide tolerance traits comprise: for tolerance to glyphosate: cp4 epsps, epsps grg23ace5, mepsps, 2mepsps, gat4601, gat4621, goxv247; for tolerance to glufosinate: pat and bar, for tolerance to 2,4-D: aad-1, aad-12; for tolerance to dicamba: dmo; for tolerance to oxynil herbicies: bxn; for tolerance to sulfonylurea herbicides: zm-hra, csr1-2, gm-hra, S4-HrA; for tolerance to ALS inhibitors: csr1-2; and for tolerance to HPPD inhibitors: hppdPF, W336, avhppd-03.

Transgenic corn events comprising herbicide tolerance genes include, but are not limited to, DAS40278, MON801, MON802, MON809, MON810, MON832, MON87411, MON87419, MON87427, MON88017, MON89034, NK603, GA21, MZHG0JG, HCEM485, VCO-Ø1981-5, 676, 678, 680, 33121, 4114, 59122, 98140, Bt10, Bt176, CBH-351, DBT418, DLL25, MS3, MS6, MZIR098, T25, TC1507 and TC_{6275.} Transgenic soybean events comprising herbicide tolerance genes include, but are not limited to, GTS 40-3-2, MON87705, MON87708, MON87712, MON87769, MON89788, A2704-12, A2704-21, A5547-127, A5547-35, DP356043, DAS44406-6, DAS68416-4, DAS-81419-2, GU262, SYHTØH2, W62, W98, FG72 and CV127. Transgenic cotton events comprising herbicide tolerance genes include, but are not limited to, 19-51a, 31707, 42317, 81910, 281-24-236, 3006-210-23, BXN10211, BXN10215, BXN10222, BXN10224, MON1445, MON1698, MON88701, MON88913, GHB119, GHB614, LLCotton25, T303-3 and T304-40. Transgenic canola events comprising herbicide tolerance genes are for example, but not excluding others, MON88302, HCR-1, HCN10, HCN28, HCN92, MS1, MS8, PHY14, PHY23, PHY35, PHY36, RF1, RF2 and RF3.

Insect resistance has mainly been created by transferring bacterial genes for insecticidal proteins to plants: Transgenes which have most frequently been used are toxin genes of Bacillus spp. and synthetic variants thereof, like cry1A, cry1Ab, cry1Ab-Ac, cry1Ac, cry1A.105, cry1F, cry1Fa2, cry2Ab2, cry2Ae, mcry3A, ecry3.1Ab, cry3Bb1, cry34Ab1, cry35Ab1, cry9C, vip3A(a), vip3Aa20. However, also genes of plant origin, such as genes coding for protease inhibitors, like CpTI and pinII, have been transferred to other plants. A further approach uses transgenes such as dvsnf7 to produce double-stranded RNA in plants.

Transgenic corn events comprising genes for insecticidal proteins or double stranded RNA include, but are not limited to, Bt10, Bt11, Bt176, MON801, MON802, MON809, MON810, MON863, MON87411, MON88017, MON89034, 33121, 4114, 5307, 59122, TC1507, TC6275, CBH-351, MIR162, DBT418 and MZIR098. Transgenic soybean events comprising genes for insecticidal proteins include, but are not limited to, MON87701, MON87751 and DAS-81419. Transgenic cotton events comprising genes for insecticidal proteins include, but are not limited to, SGK321, MON531, MON757, MON1076, MON15985, 31707, 31803, 31807, 31808, 42317, BNLA-601, Event1, COT67B, COT102, T303-3, T304-40, GFM Cry1A, GK12, MLS 9124, 281-24-236, 3006-210-23, GHB119 and SGK321.

Increased yield has been created by using the transgene athb17, being present for example in corn event MON87403, or by using the transgene bbx32, being present for example in the soybean event MON87712.

Cultivated plants comprising a modified oil content have been created by using the transgenes: gm-fad2-1, Pj.D6D, Nc.Fad3, fad2-1A and fatb1-A. Soybean events comprising at least one of these genes are: 260-05, MON87705 and MON87769.

Tolerance to abiotic conditions, such as drought, has been created by using the transgene cspB, comprised by the corn event MON87460 and by using the transgene Hahb-4, comprised by soybean event IND-ØØ41Ø-5.

Traits are frequently combined by combining genes in a transformation event or by combining different events during the breeding process resulting in a cultivated plant with stacked traits. Preferred combinations of traits are combinations of herbicide tolerance traits to different groups of herbicides, combinations of insect tolerance to different kind of insects, in particular tolerance to lepidopteran and coleopteran insects, combinations of herbicide tolerance with one or several types of insect resistance, combinations of herbicide tolerance with increased yield as well as combinations of herbicide tolerance and tolerance to abiotic conditions.

Plants comprising singular or stacked traits as well as the genes and events providing these traits are well known in the art. For example, detailed information as to the mutagenized or integrated genes and the respective events are available from websites of the organizations “International Service for the Acquisition of Agri-biotech Applications (ISAAA)” (http://www.isaaa.org/gmapprovaldatabase) and the “Center for Environmental Risk Assessment (CERA)” (http://cera-gmc.org/GMCropDatabase). Further information on specific events and methods to detect them can be found for canola events MS1, MS8, RF3, GT73, MON88302, KK179 in WO01/031042, WO01/041558, WO01/041558, WO02/036831, WO11/153186, WO13/003558, for cotton events MON1445, MON15985, MON531 (MON15985), LLCotton25, MON88913, COT102, 281-24-236, 3006-210-23, COT67B, GHB614, T304-40, GHB119, MON88701, 81910 in WO02/034946, WO02/100163, WO02/100163, WO03/013224, WO04/072235, WO04/039986, WO05/103266, WO05/103266, WO06/128573, WO07/017186, WO08/122406, WO08/151780, WO12/134808, WO13/112527; for corn events GA21, MON810, DLL25, TC1507, MON863, MIR604, LY038, MON88017, 3272, 59122, NK603, MIR162, MON89034, 98140, 32138, MON87460, 5307, 4114, MON87427, DAS40278, MON87411, 33121, MON87403, MON87419 in WO98/044140, U.S. Ser. No. 02/102,582, U.S. Ser. No. 03/126634, WO04/099447, WO04/011601, WO05/103301, WO05/061720, WO05/059103, WO06/098952, WO06/039376, US2007/292854, WO07/142840, WO07/140256, WO08/112019, WO09/103049, WO09/111263, WO10/077816, WO11/084621, WO11/062904, WO11/022469, WO13/169923, WO14/116854, WO15/053998, WO15/142571; for potato events E12, F10, J3, J55, V11, X17, Y9 in WO14/178910, WO14/178913, WO14/178941, WO14/179276, WO16/183445, WO17/062831, WO17/062825; for rice events LLRICE06, LLRICE601, LLRICE62 in WO00/026345, WO00/026356, WO00/026345; and for soybean events H7-1, MON89788, A2704-12, A5547-127, DP305423, DP356043, MON87701, MON87769, CV127, MON87705, DAS68416-4, MON87708, MON87712, SYHT0H2, DAS81419, DAS81419×DAS44406-6, MON87751 in WO04/074492, WO06/130436, WO06/108674, WO06/108675, WO08/054747, WO08/002872, WO09/064652, WO09/102873, WO10/080829, WO10/037016, WO11/066384, WO11/034704, WO12/051199, WO12/082548, WO13/016527, WO13/016516, WO14/201235.

The use of compounds I and compositions according to the invention, respectively, on cultivated plants may result in effects which are specific to a cultivated plant comprising a certain gene or event. These effects might involve changes in growth behavior or changed resistance to biotic or abiotic stress factors. Such effects may in particular comprise enhanced yield, enhanced resistance or tolerance to insects, nematodes, fungal, bacterial, mycoplasma, viral or viroid pathogens as well as early vigour, early or delayed ripening, cold or heat tolerance as well as changed amino acid or fatty acid spectrum or content.

The compounds I and compositions thereof, respectively, are particularly suitable for controlling the following plant diseases:

Albugo spp. (white rust) on ornamentals, vegetables (e. g. A. candida) and sunflowers (e. g. A. tragopogonis); Alternaria spp. (Alternaria leaf spot) on vegetables, rape (A. brassicola or brassicae), sugar beets (A. tenuis), fruits, rice, soybeans, potatoes (e. g. A. solani or A. alternata), tomatoes (e. g. A. solani or A. alternata) and wheat; Aphanomyces spp. on sugar beets and vegetables; Ascochyta spp. on cereals and vegetables, e. g. A. tritici (anthracnose) on wheat and A. hordei on barley; Bipolaris and Drechslera spp. (teleomorph: Cochliobolus spp.), e. g. Southern leaf blight (D. maydis) or Northern leaf blight (B. zeicola) on corn, e. g. spot blotch (B. sorokiniana) on cereals and e. g. B. oryzae on rice and turfs; Blumeria (formerly Eysiphe) graminis (powdery mildew) on cereals (e. g. on wheat or barley); Botrytis cinerea (teleomorph: Botryotinia fuckeliana: grey mold) on fruits and berries (e. g. strawberries), vegetables (e. g. lettuce, carrots, celery and cabbages), rape, flowers, vines, forestry plants and wheat; Bremia lactucae (downy mildew) on lettuce; Ceratocystis (syn. Ophiostoma) spp. (rot or wilt) on broad-leaved trees and evergreens, e. g. C. ulmi (Dutch elm disease) on elms; Cercospora spp. (Cercospora leaf spots) on corn (e. g. Gray leaf spot: C. zeae-maydis), rice, sugar beets (e. g. C. beticola), sugar cane, vegetables, coffee, soybeans (e. g. C. sojina or C. kikuchii) and rice; Cladosporium spp. on tomatoes (e. g. C. fulvum: leaf mold) and cereals, e. g. C. herbarum (black ear) on wheat; Claviceps purpurea (ergot) on cereals; Cochliobolus (anamorph: Helminthosporium of Bipolaris) spp. (leaf spots) on corn (C. carbonum), cereals (e. g. C. sativus, anamorph: B. sorokiniana) and rice (e. g. C. miyabeanus, anamorph: H. oryzae); Colletotrichum (teleomorph: Glomerella) spp. (anthracnose) on cotton (e. g. C. gossypii), corn (e. g. C. graminicola: Anthracnose stalk rot), soft fruits, potatoes (e. g. C. coccodes: black dot), beans (e. g. C. lindemuthianum) and soybeans (e. g. C. truncatum or C. gloeosporioides); Corticium spp., e. g. C. sasakii (sheath blight) on rice; Corynespora cassiicola (leaf spots) on soybeans and ornamentals; Cycloconium spp., e. g. C. oleaginum on olive trees; Cylindrocarpon spp. (e. g. fruit tree canker or young vine decline, teleomorph: Nectria or Neonectria spp.) on fruit trees, vines (e. g. C. liriodendri, teleomorph: Neonectria liriodendri: Black Foot Disease) and ornamentals; Dematophora (teleomorph: Rosellinia) necatrix (root and stem rot) on soybeans; Diaporthe spp., e. g. D. phaseolorum (damping off) on soybeans; Drechslera (syn. Helminthosporium, teleomorph: Pyrenophora) spp. on corn, cereals, such as barley (e. g. D. teres, net blotch) and wheat (e. g. D. tritici-repentis: tan spot), rice and turf; Esca (dieback, apoplexy) on vines, caused by Formifiporia (syn. Phellinus) punctata, F. mediterranea, Phaeomoniella chlamydospora (earlier Phaeoacremonium chlamydosporum), Phaeoacremonium aleophilum and/or Botryosphaeria obtusa; Elsinoe spp. on pome fruits (E. pyri), soft fruits (E. veneta: anthracnose) and vines (E. ampelina: anthracnose); Entyloma oryzae (leaf smut) on rice; Epicoccum spp. (black mold) on wheat; Elysiphe spp. (powdery mildew) on sugar beets (E. betae), vegetables (e. g. E. pisi), such as cucurbits (e. g. E. cichoracearum), cabbages, rape (e. g. E. cruciferarum); Eutypa lata (Eutypa canker or dieback, anamorph: Cytosporina lata, syn. Libertella blepharis) on fruit trees, vines and ornamental woods; Exserohilum (syn. Helminthosporium) spp. on corn (e. g. E. turcicum); Fusarium (teleomorph: Gibberella) spp. (wilt, root or stem rot) on various plants, such as F. graminearum or F. culmorum (root rot, scab or head blight) on cereals (e. g. wheat or barley), F. oxysporum on tomatoes, F. solani (f. sp. glycines now syn. F. virguliforme) and F. tucumaniae and F. brasiliense each causing sudden death syndrome on soybeans, and F. verticillioides on corn; Gaeumannomyces graminis (take-all) on cereals (e. g. wheat or barley) and corn; Gibberella spp. on cereals (e. g. G. zeae) and rice (e. g. G. fujikuroi: Bakanae disease); Glomerella cingulata on vines, pome fruits and other plants and G. gossypii on cotton; Grainstaining complex on rice; Guignardia bewellii (black rot) on vines; Gymnosporangium spp. on rosaceous plants and junipers, e. g. G. sabinae (rust) on pears; Helminthosporium spp. (syn. Drechslera, teleomorph: Cochliobolus) on corn, cereals and rice; Hemileia spp., e. g. H. vastatrix (coffee leaf rust) on coffee; Isariopsis clavispora (syn. Cladosporium vitis) on vines; Macrophomina phaseolina (syn. phaseoli) (root and stem rot) on soybeans and cotton; Microdochium (syn. Fusarium) nivale (pink snow mold) on cereals (e. g. wheat or barley); Microsphaera diffusa (powdery mildew) on soybeans; Monilinia spp., e. g. M. laxa, M. fructicola and M. fructigena (bloom and twig blight, brown rot) on stone fruits and other rosaceous plants; Mycosphaerella spp. on cereals, bananas, soft fruits and ground nuts, such as e. g. M. graminicola (anamorph: Septoria tritici, Septoria blotch) on wheat or M. fijiensis (black Sigatoka disease) on bananas; Peronospora spp. (downy mildew) on cabbage (e. g. P. brassicae), rape (e. g. P. parasitica), onions (e. g. P. destructor), tobacco (P. tabacina) and soybeans (e. g. P. manshurica); Phakopsora pachyrhizi and P. meibomiae (soybean rust) on soybeans; Phialophora spp. e. g. on vines (e. g. P. tracheiphila and P. tetraspora) and soybeans (e. g. P. gregata: stem rot); Phoma lingam (root and stem rot) on rape and cabbage and P. betae (root rot, leaf spot and damping-off) on sugar beets; Phomopsis spp. on sunflowers, vines (e. g. P. viticola: can and leaf spot) and soybeans (e. g. stem rot: P. phaseoli, teleomorph: Diaporthe phaseolorum); Physoderma maydis (brown spots) on corn; Phytophthora spp. (wilt, root, leaf, fruit and stem root) on various plants, such as paprika and cucurbits (e. g. P. capsici), soybeans (e. g. P. megasperma, syn. P. sojae), potatoes and tomatoes (e. g. P. infestans: late blight) and broad-leaved trees (e. g. P. ramorum: sudden oak death); Plasmodiophora brassicae (club root) on cabbage, rape, radish and other plants; Plasmopara spp., e. g. P. viticola (grapevine downy mildew) on vines and P. halstedii on sunflowers; Podosphaera spp. (powdery mildew) on rosaceous plants, hop, pome and soft fruits, e. g. P. leucotricha on apples; Polymyxa spp., e. g. on cereals, such as barley and wheat (P. graminis) and sugar beets (P. betae) and thereby transmitted viral diseases; Pseudocercosporella herpotrichoides (eyespot, teleomorph: Tapesia yallundae) on cereals, e. g. wheat or barley; Pseudoperonospora (downy mildew) on various plants, e. g. P. cubensis on cucurbits or P. humili on hop; Pseudopezicula tracheiphila (red fire disease or ‘rotbrenner’, anamorph: Phialophora) on vines; Puccinia spp. (rusts) on various plants, e. g. P. triticina (brown or leaf rust), P. striiformis (stripe or yellow rust), P. hordei (dwarf rust), P. graminis (stem or black rust) or P. recondita (brown or leaf rust) on cereals, such as e. g. wheat, barley or rye, P. kuehnii (orange rust) on sugar cane and P. asparagi on asparagus; Pyrenophora (anamorph: Drechslera) tritici-repentis (tan spot) on wheat or P. teres (net blotch) on barley; Pyricularia spp., e. g. P. oryzae (teleomorph: Magnaporthe grisea, rice blast) on rice and P. grisea on turf and cereals; Pythium spp. (damping-off) on turf, rice, corn, wheat, cotton, rape, sunflowers, soybeans, sugar beets, vegetables and various other plants (e. g. P. ultimum or P. aphanidermatum); Ramularia spp., e. g. R. collo-cygni (Ramularia leaf spots, Physiological leaf spots) on barley and R. beticola on sugar beets; Rhizoctonia spp. on cotton, rice, potatoes, turf, corn, rape, potatoes, sugar beets, vegetables and various other plants, e. g. R. solani (root and stem rot) on soybeans, R. solani (sheath blight) on rice or R. cerealis (Rhizoctonia spring blight) on wheat or barley; Rhizopus stolonifer (black mold, soft rot) on strawberries, carrots, cabbage, vines and tomatoes; Rhynchosporium secalis (scald) on barley, rye and triticale; Sarocladium oryzae and S. attenuatum (sheath rot) on rice; Sclerotinia spp. (stem rot or white mold) on vegetables and field crops, such as rape, sunflowers (e. g. S. sclerotiorum) and soybeans (e. g. S. rolfsii or S. sclerotiorum); Septoria spp. on various plants, e. g. S. glycines (brown spot) on soybeans, S. tritici (Septoria blotch) on wheat and S. (syn. Stagonospora) nodorum (Stagonospora blotch) on cereals; Uncinula (syn. Erysiphe) necator(powdery mildew, anamorph: Oidium tuckeri) on vines; Setospaeria spp. (leaf blight) on corn (e. g. S. turcicum, syn. Helminthosporium turcicum) and turf; Sphacelotheca spp. (smut) on corn, (e. g. S. reiliana: head smut), sorghum and sugar cane; Sphaerotheca fuliginea (powdery mildew) on cucurbits; Spongospora subterranea (powdery scab) on potatoes and thereby transmitted viral diseases; Stagonospora spp. on cereals, e. g. S. nodorum (Stagonospora blotch, teleomorph: Leptosphaeria [syn. Phaeosphaeria] nodorum) on wheat; Synchytrium endobioticum on potatoes (potato wart disease); Taphrina spp., e. g. T. deformans (leaf curl disease) on peaches and T. pruni (plum pocket) on plums; Thielaviopsis spp. (black root rot) on tobacco, pome fruits, vegetables, soybeans and cotton, e. g. T. basicola (syn. Chalara elegans); Tilletia spp. (common bunt or stinking smut) on cereals, such as e. g. T. tritici (syn. T. caries, wheat bunt) and T. controversa (dwarf bunt) on wheat; Typhula incarnata (grey snow mold) on barley or wheat; Urocystis spp., e. g. U. occulta (stem smut) on rye; Uromyces spp. (rust) on vegetables, such as beans (e. g. U. appendiculatus, syn. U. phaseoli) and sugar beets (e. g. U. betae); Ustilago spp. (loose smut) on cereals (e. g. U. nuda and U. avaenae), corn (e. g. U. maydis: corn smut) and sugar cane; Venturia spp. (scab) on apples (e. g. V. inaequalis) and pears; and Verticillium spp. (wilt) on various plants, such as fruits and ornamentals, vines, soft fruits, vegetables and field crops, e. g. V. dahliae on strawberries, rape, potatoes and tomatoes. In a preferred embodiment the compounds I and compositions thereof, respectively, are particularly suitable for controlling the following plant diseases: Puccinia spp. (rusts) on various plants, for example, but not limited to P. triticina (brown or leaf rust), P. striiformis (stripe or yellow rust), P. hordei (dwarf rust), P. graminis (stem or black rust) or P. recondita (brown or leaf rust) on cereals, such as e. g. wheat, barley or rye and Phakopsoraceae spp. on various plants, in particular Phakopsora pachyrhizi and P. melbomiae (soybean rust) on soybeans. The compounds I and compositions thereof, respectively, are also suitable for controlling harmful fungi in the protection of stored products or harvest and in the protection of materials. The term “protection of materials” is to be understood to denote the protection of technical and non-living materials, such as adhesives, glues, wood, paper and paperboard, textiles, leather, paint dispersions, plastics, cooling lubricants, fiber or fabrics, against the infestation and destruction by harmful microorganisms, such as fungi and bacteria. As to the protection of wood and other materials, the particular attention is paid to the following harmful fungi: Ascomycetes such as Ophiostoma spp., Ceratocystis spp., Aureobasidium pullulans, Sclerophoma spp., Chaetomium spp., Humicola spp., Petriella spp., Trichurus spp.; Basidiomycetes such as Coniophora spp., Coriolus spp., Gloeophyllum spp., Lentinus spp., Pleurotus spp., Poria spp., Serpula spp. and Tyromyces spp., Deuteromycetes such as Aspergillus spp., Cladosporium spp., Penicillium spp., Trichoderma spp., Alternaria spp., Paecilomyces spp. and Zygomycetes such as Mucor spp., and in addition in the protection of stored products and harvest the following yeast fungi are worthy of note: Candida spp. and Saccharomyces cerevisae. The method of treatment according to the invention can also be used in the field of protecting stored products or harvest against attack of fungi and microorganisms. According to the present invention, the term “stored products” is understood to denote natural substances of plant or animal origin and their processed forms, which have been taken from the natural life cycle and for which long-term protection is desired. Stored products of crop plant origin, such as plants or parts thereof, for example stalks, leafs, tubers, seeds, fruits or grains, can be protected in the freshly harvested state or in processed form, such as pre-dried, moistened, comminuted, ground, pressed or roasted, which process is also known as post-harvest treatment. Also falling under the definition of stored products is timber, whether in the form of crude timber, such as construction timber, electricity pylons and barriers, or in the form of finished articles, such as furniture or objects made from wood. Stored products of animal origin are hides, leather, furs, hairs and the like. The combinations according the present invention can prevent disadvantageous effects such as decay, discoloration or mold. Preferably “stored products” is understood to denote natural substances of plant origin and their processed forms, more preferably fruits and their processed forms, such as pomes, stone fruits, soft fruits and citrus fruits and their processed forms.

The compounds I and compositions thereof, respectively, may be used for improving the health of a plant. The invention also relates to a method for improving plant health by treating a plant, its propagation material and/or the locus where the plant is growing or is to grow with an effective amount of compounds I and compositions thereof, respectively.

The term “plant health” is to be understood to denote a condition of the plant and/or its products which is determined by several indicators alone or in combination with each other such as yield (e. g. increased biomass and/or increased content of valuable ingredients), plant vigor (e. g. improved plant growth and/or greener leaves (“greening effect”)), quality (e. g. improved content or composition of certain ingredients) and tolerance to abiotic and/or biotic stress. The above identified indicators for the health condition of a plant may be interdependent or may result from each other.

The compounds of formula I can be present in different crystal modifications whose biological activity may differ. They are likewise subject matter of the present invention.

The compounds I are employed as such or in form of compositions by treating the fungi or the plants, plant propagation materials, such as seeds, soil, surfaces, materials or rooms to be protected from fungal attack with a fungicidally effective amount of the active substances. The application can be carried out both before and after the infection of the plants, plant propagation materials, such as seeds, soil, surfaces, materials or rooms by the fungi.

Plant propagation materials may be treated with compounds I as such or a composition comprising at least one compound I prophylactically either at or before planting or transplanting. The invention also relates to agrochemical compositions comprising an auxiliary and at least one compound I according to the invention.

An agrochemical composition comprises a fungicidally effective amount of a compound I. The term “effective amount” denotes an amount of the composition or of the compounds I, which is sufficient for controlling harmful fungi on cultivated plants or in the protection of materials and which does not result in a substantial damage to the treated plants. Such an amount can vary in a broad range and is dependent on various factors, such as the fungal species to be controlled, the treated cultivated plant or material, the climatic conditions and the specific compound I used.

The compounds I, their N-oxides and salts can be converted into customary types of agrochemical compositions, e. g. solutions, emulsions, suspensions, dusts, powders, pastes, granules, pressings, capsules, and mixtures thereof. Examples for composition types are suspensions (e. g. SC, OD, FS), emulsifiable concentrates (e. g. EC), emulsions (e. g. EW, EO, ES, ME), capsules (e. g. CS, ZC), pastes, pastilles, wettable powders or dusts (e. g. WP, SP, WS, DP, DS), pressings (e. g. BR, TB, DT), granules (e. g. WG, SG, GR, FG, GG, MG), insecticidal articles (e. g. LN), as well as gel formulations for the treatment of plant propagation materials such as seeds (e. g. GF). These and further compositions types are defined in the “Catalogue of pesticide formulation types and international coding system”, Technical Monograph No. 2, 6^{th} Ed. May 2008, CropLife International. The compositions are prepared in a known manner, such as described by Mollet and Grubemann, Formulation technology, Wiley VCH, Weinheim, 2001; or Knowles, New developments in crop protection product formulation, Agrow Reports DS243, T&F Informa, London, 2005.

Suitable auxiliaries are solvents, liquid carriers, solid carriers or fillers, surfactants, dispersants, emulsifiers, wetters, adjuvants, solubilizers, penetration enhancers, protective colloids, adhesion agents, thickeners, humectants, repellents, attractants, feeding stimulants, compatibilizers, bactericides, anti-freezing agents, anti-foaming agents, colorants, tackifiers and binders.

Suitable solvents and liquid carriers are water and organic solvents, such as mineral oil fractions of medium to high boiling point, e. g. kerosene, diesel oil; oils of vegetable or animal origin; aliphatic, cyclic and aromatic hydrocarbons, e. g. toluene, paraffin, tetrahydronaphthalene, alkylated naphthalenes; alcohols, e. g. ethanol, propanol, butanol, benzyl alcohol, cyclohexanol; glycols; DMSO; ketones, e. g. cyclohexanone; esters, e. g. lactates, carbonates, fatty acid esters, gamma-butyrolactone; fatty acids; phosphonates; amines; amides, e. g. N-methyl pyrrolidone, fatty acid dimethyl amides; and mixtures thereof. Suitable solid carriers or fillers are mineral earths, e. g. silicates, silica gels, talc, kaolins, limestone, lime, chalk, clays, dolomite, diatomaceous earth, bentonite, calcium sulfate, magnesium sulfate, magnesium oxide; polysaccharides, e. g. cellulose, starch; fertilizers, e. g. ammonium sulfate, ammonium phosphate, ammonium nitrate, ureas; products of vegetable origin, e. g. cereal meal, tree bark meal, wood meal, nutshell meal, and mixtures thereof. Suitable surfactants are surface-active compounds, such as anionic, cationic, nonionic and amphoteric surfactants, block polymers, polyelectrolytes, and mixtures thereof. Such surfactants can be used as emulsifier, dispersant, solubilizer, wetter, penetration enhancer, protective colloid, or adjuvant. Examples of surfactants are listed in McCutcheon's, Vol. 1: Emulsifiers & Detergents, McCutcheon's Directories, Glen Rock, USA, 2008 (International Ed. or North American Ed.).

Suitable anionic surfactants are alkali, alkaline earth or ammonium salts of sulfonates, sulfates, phosphates, carboxylates, and mixtures thereof. Examples of sulfonates are alkylaryl sulfonates, diphenyl sulfonates, alpha-olefin sulfonates, lignin sulfonates, sulfonates of fatty acids and oils, sulfonates of ethoxylated alkylphenols, sulfonates of alkoxylated arylphenols, sulfonates of condensed naphthalenes, sulfonates of dodecyl- and tridecylbenzenes, sulfonates of naphthalenes and alkyl naphthalenes, sulfosuccinates or sulfosuccinamates. Examples of sulfates are sulfates of fatty acids and oils, of ethoxylated alkylphenols, of alcohols, of ethoxylated alcohols, or of fatty acid esters. Examples of phosphates are phosphate esters. Examples of carboxylates are alkyl carboxylates, and carboxylated alcohol or alkylphenol ethoxylates.

Suitable nonionic surfactants are alkoxylates, N-substituted fatty acid amides, amine oxides, esters, sugar-based surfactants, polymeric surfactants, and mixtures thereof. Examples of alkoxylates are compounds such as alcohols, alkylphenols, amines, amides, arylphenols, fatty acids or fatty acid esters which have been alkoxylated with 1 to 50 equivalents. Ethylene oxide and/or propylene oxide may be employed for the alkoxylation, preferably ethylene oxide. Examples of N-substituted fatty acid amides are fatty acid glucamides or fatty acid alkanolamides. Examples of esters are fatty acid esters, glycerol esters or monoglycerides. Examples of sugar-based surfactants are sorbitans, ethoxylated sorbitans, sucrose and glucose esters or alkylpolyglucosides. Examples of polymeric surfactants are home- or copolymers of vinyl pyrrolidone, vinyl alcohols, or vinyl acetate.

Suitable cationic surfactants are quaternary surfactants, for example quaternary ammonium compounds with one or two hydrophobic groups, or salts of long-chain primary amines. Suitable amphoteric surfactants are alkylbetains and imidazolines. Suitable block polymers are block polymers of the A-B or A-B-A type comprising blocks of polyethylene oxide and polypropylene oxide, or of the A-B-C type comprising alkanol, polyethylene oxide and polypropylene oxide. Suitable polyelectrolytes are polyacids or polybases. Examples of polyacids are alkali salts of polyacrylic acid or polyacid comb polymers. Examples of polybases are polyvinyl amines or polyethylene amines.

Suitable adjuvants are compounds, which have a negligible or even no pesticidal activity themselves, and which improve the biological performance of the compound I on the target. Examples are surfactants, mineral or vegetable oils, and other auxiliaries. Further examples are listed by Knowles, Adjuvants and additives, Agrow Reports DS256, T&F Informa UK, 2006, chapter 5.

Suitable thickeners are polysaccharides (e. g. xanthan gum, carboxymethyl cellulose), inorganic clays (organically modified or unmodified), polycarboxylates, and silicates.

Suitable bactericides are bronopol and isothiazolinone derivatives such as alkylisothiazolinones and benzisothiazolinones.

Suitable anti-freezing agents are ethylene glycol, propylene glycol, urea and glycerin.

Suitable anti-foaming agents are silicones, long chain alcohols, and salts of fatty acids.

Suitable colorants (e. g. in red, blue, or green) are pigments of low water solubility and water-soluble dyes. Examples are inorganic colorants (e. g. iron oxide, titan oxide, iron hexacyanoferrate) and organic colorants (e. g. alizarin-, azo- and phthalocyanine colorants). Suitable tackifiers or binders are polyvinyl pyrrolidones, polyvinyl acetates, polyvinyl alcohols, polyacrylates, biological or synthetic waxes, and cellulose ethers.

Examples for composition types and their preparation are:

i) Water-soluble concentrates (SL, LS)

10-60 wt % of a compound I and 5-15 wt % wetting agent (e. g. alcohol alkoxylates) are dissolved in water and/or in a water-soluble solvent (e. g. alcohols) ad 100 wt %. The active substance dissolves upon dilution with water.

ii) Dispersible concentrates (DC)

5-25 wt % of a compound I and 1-10 wt % dispersant (e. g. polyvinyl pyrrolidone) are dissolved in organic solvent (e. g. cyclohexanone) ad 100 wt %. Dilution with water gives a dispersion.

iii) Emulsifiable concentrates (EC)

15-70 wt % of a compound I and 5-10 wt % emulsifiers (e. g. calcium dodecylbenzenesulfonate and castor oil ethoxylate) are dissolved in water-insoluble organic solvent (e. g. aromatic hydrocarbon) ad 100 wt %. Dilution with water gives an emulsion.

iv) Emulsions (EW, EO, ES)

5-40 wt % of a compound I and 1-10 wt % emulsifiers (e. g. calcium dodecylbenzenesulfonate and castor oil ethoxylate) are dissolved in 20-40 wt % water-insoluble organic solvent (e. g. aromatic hydrocarbon). This mixture is introduced into water ad 100 wt % by means of an emulsifying machine and made into a homogeneous emulsion. Dilution with water gives an emulsion.

v) Suspensions (SC, OD, FS)

In an agitated ball mill, 20-60 wt % of a compound I are comminuted with addition of 2-10 wt % dispersants and wetting agents (e. g. sodium lignosulfonate and alcohol ethoxylate), 0.1-2 wt % thickener (e. g. xanthan gum) and water ad 100 wt % to give a fine active substance suspension. Dilution with water gives a stable suspension of the active substance. For FS type composition up to 40 wt % binder (e. g. polyvinyl alcohol) is added.

vi) Water-dispersible granules and water-soluble granules (WG, SG)

50-80 wt % of a compound I are ground finely with addition of dispersants and wetting agents (e. g. sodium lignosulfonate and alcohol ethoxylate) ad 100 wt % and prepared as water-dispersible or water-soluble granules by means of technical appliances (e. g. extrusion, spray tower, fluidized bed). Dilution with water gives a stable dispersion or solution of the active substance.

vii) Water-dispersible powders and water-soluble powders (WP, SP, WS)

50-80 wt % of a compound I are ground in a rotor-stator mill with addition of 1-5 wt % dispersants (e. g. sodium lignosulfonate), 1-3 wt % wetting agents (e. g. alcohol ethoxylate) and solid carrier (e. g. silica gel) ad 100 wt %. Dilution with water gives a stable dispersion or solution of the active substance.

viii) Gel (GW, GF)

In an agitated ball mill, 5-25 wt % of a compound I are comminuted with addition of 3-10 wt % dispersants (e. g. sodium lignosulfonate), 1-5 wt % thickener (e. g. carboxymethyl cellulose) and water ad 100 wt % to give a fine suspension of the active substance. Dilution with water gives a stable suspension of the active substance.

ix) Microemulsion (ME)

5-20 wt % of a compound I are added to 5-30 wt % organic solvent blend (e. g. fatty acid dimethyl amide and cyclohexanone), 10-25 wt % surfactant blend (e. g. alcohol ethoxylate and arylphenol ethoxylate), and water ad 100%. This mixture is stirred for 1 h to produce spontaneously a thermodynamically stable microemulsion.

x) Microcapsules (CS)

An oil phase comprising 5-50 wt % of a compound I, 0-40 wt % water insoluble organic solvent (e. g. aromatic hydrocarbon), 2-15 wt % acrylic monomers (e. g. methylmethacrylate, methacrylic acid and a di- or triacrylate) are dispersed into an aqueous solution of a protective colloid (e. g. polyvinyl alcohol). Radical polymerization results in the formation of poly(meth)acrylate microcapsules. Alternatively, an oil phase comprising 5-50 wt % of a compound I according to the invention, 0-40 wt % water insoluble organic solvent (e. g. aromatic hydrocarbon), and an isocyanate monomer (e. g. diphenylmethene-4,4′-diisocyanatae) are dispersed into an aqueous solution of a protective colloid (e. g. polyvinyl alcohol). The addition of a polyamine (e. g. hexamethylenediamine) results in the formation of polyurea microcapsules. The monomers amount to 1-10 wt %. The wt % relate to the total CS composition.

xi) Dustable powders (DP, DS)

1-10 wt % of a compound I are ground finely and mixed intimately with solid carrier (e. g. finely divided kaolin) ad 100 wt %.

xii) Granules (GR, FG)

0.5-30 wt % of a compound I is ground finely and associated with solid carrier (e. g. silicate) ad 100 wt %. Granulation is achieved by extrusion, spray-drying or fluidized bed.

xiii) Ultra-low volume liquids (UL)

1-50 wt % of a compound I are dissolved in organic solvent (e. g. aromatic hydrocarbon) ad 100 wt %.

The compositions types i) to xiii) may optionally comprise further auxiliaries, such as 0.1-1 wt % bactericides, 5-15 wt % anti-freezing agents, 0.1-1 wt % anti-foaming agents, and 0.1-1 wt % colorants.

The agrochemical compositions generally comprise between 0.01 and 95%, preferably between 0.1 and 90%, more preferably between 1 and 70%, and in particular between 10 and 60%, by weight of active substance. The active substances are employed in a purity of from 90% to 100%, preferably from 95% to 100% (according to NMR spectrum).

For the purposes of treatment of plant propagation materials, particularly seeds, solutions for seed treatment (LS), Suspoemulsions (SE), flowable concentrates (FS), powders for dry treatment (DS), water-dispersible powders for slurry treatment (WS), water-soluble powders (SS), emulsions (ES), emulsifiable concentrates (EC), and gels (GF) are usually employed. The compositions in question give, after two-to-tenfold dilution, active substance concentrations of from 0.01 to 60% by weight, preferably from 0.1 to 40%, in the ready-to-use preparations. Application can be carried out before or during sowing. Methods for applying compound I and compositions thereof, respectively, onto plant propagation material, especially seeds, include dressing, coating, pelleting, dusting, and soaking as well as in-furrow application methods. Preferably, compound I or the compositions thereof, respectively, are applied on to the plant propagation material by a method such that germination is not induced, e. g. by seed dressing, pelleting, coating and dusting.

When employed in plant protection, the amounts of active substances applied are, depending on the kind of effect desired, from 0.001 to 2 kg per ha, preferably from 0.005 to 2 kg per ha, more preferably from 0.05 to 0.9 kg per ha, and in particular from 0.1 to 0.75 kg per ha. In treatment of plant propagation materials such as seeds, e. g. by dusting, coating or drenching seed, amounts of active substance of from 0.1 to 1000 g, preferably from 1 to 1000 g, more preferably from 1 to 100 g and most preferably from 5 to 100 g, per 100 kilogram of plant propagation material (preferably seeds) are generally required.

When used in the protection of materials or stored products, the amount of active substance applied depends on the kind of application area and on the desired effect. Amounts customarily applied in the protection of materials are 0.001 g to 2 kg, preferably 0.005 g to 1 kg, of active substance per cubic meter of treated material.

Various types of oils, wetters, adjuvants, fertilizer, or micronutrients, and further pesticides (e. g. herbicides, insecticides, fungicides, growth regulators, safeners, biopesticides) may be added to the active substances or the compositions comprising them as premix or, if appropriate not until immediately prior to use (tank mix). These agents can be admixed with the compositions according to the invention in a weight ratio of 1:100 to 100:1, preferably 1:10 to 10:1.

A pesticide is generally a chemical or biological agent (such as pestidal active ingredient, compound, composition, virus, bacterium, antimicrobial or disinfectant) that through its effect deters, incapacitates, kills or otherwise discourages pests. Target pests can include insects, plant pathogens, weeds, mollusks, birds, mammals, fish, nematodes (roundworms), and microbes that destroy property, cause nuisance, spread disease or are vectors for disease. The term “pesticide” includes also plant growth regulators that alter the expected growth, flowering, or reproduction rate of plants; defoliants that cause leaves or other foliage to drop from a plant, usually to facilitate harvest; desiccants that promote drying of living tissues, such as unwanted plant tops; plant activators that activate plant physiology for defense of against certain pests; safeners that reduce unwanted herbicidal action of pesticides on crop plants; and plant growth promoters that affect plant physiology e.g. to increase plant growth, biomass, yield or any other quality parameter of the harvestable goods of a crop plant.

The user applies the composition according to the invention usually from a predosage device, a knapsack sprayer, a spray tank, a spray plane, or an irrigation system. Usually, the agrochemical composition is made up with water, buffer, and/or further auxiliaries to the desired application concentration and the ready-to-use spray liquor or the agrochemical composition according to the invention is thus obtained. Usually, 20 to 2000 liters, preferably 50 to 400 liters, of the ready-to-use spray liquor are applied per hectare of agricultural useful area. According to one embodiment, individual components of the composition according to the invention such as parts of a kit or parts of a binary or ternary mixture may be mixed by the user himself in a spray tank or any other kind of vessel used for applications (e. g. seed treater drums, seed pelleting machinery, knapsack sprayer) and further auxiliaries may be added, if appropriate.

Consequently, one embodiment of the invention is a kit for preparing a usable pesticidal composition, the kit comprising a) a composition comprising component 1) as defined herein and at least one auxiliary; and b) a composition comprising component 2) as defined herein and at least one auxiliary; and optionally c) a composition comprising at least one auxiliary and optionally a further active component 3) as defined herein.

Mixing the compounds I or the compositions comprising them in the use form as fungicides with other fungicides results in many cases in an expansion of the fungicidal spectrum of activity being obtained or in a prevention of fungicide resistance development. Furthermore, in many cases, synergistic effects are obtained.

The following list of pesticides II (e. g. pesticidally-active substances and biopesticides), in conjunction with which the compounds I can be used, is intended to illustrate the possible combinations but does not limit them:

A) Respiration inhibitors: Inhibitors of complex III at Qₒ site: azoxystrobin (A.1.1), coumeth-oxystrobin (A.1.2), coumoxystrobin (A.1.3), dimoxystrobin (A.1.4), enestroburin (A.1.5), fenaminstrobin (A.1.6), fenoxystrobin/flufenoxystrobin (A.1.7), fluoxastrobin (A.1.8), kresoxim-methyl (A.1.9), mandestrobin (A.1.10), metominostrobin (A.1.11), orysastrobin (A.1.12), picoxy-strobin (A.1.13), pyraclostrobin (A.1.14), pyrametostrobin (A.1.15), pyraoxystrobin (A.1.16), trifloxystrobin (A.1.17), 2-(2-(3-(2,6-dichlorophenyl)-1-methyl-allylideneaminooxymethyl)-phenyl)-2-methoxyimino-N-methyl-acetamide (A.1.18), pyribencarb (A.1.19), triclopyricarb/chlorodincarb (A.1.20), famoxadone (A.1.21), fenamidone (A.1.21), methyl-N-[2-[(1,4-dimethyl-5-phenyl-pyrazol-3-yl)oxylmethyl]phenyl]-N-methoxy-carbamate (A.1.22), 1-[2-[[1-(4-chlorophenyl)pyrazol-3-yl]oxymethyl]-3-methyl-phenyl]-4-methyl-tetrazol-5-one (A.1.25), 1-methyl-4-[3-methyl-2-[[1-[3-(trifluoromethyl)phenyl]-ethylideneamino]oxymethyl]phenyl]-tetrazol-5-one (A.1.33), (Z,2E)-5-[1-(2,4-dichlorophenyl)pyrazol-3-yl]-oxy-2-methoxyimino-N,3-dimethyl-pent-3-enamide (A.1.34), (Z2E)-5-[1-(4-chlorophenyl)pyrazol-3-yl]oxy-2-methoxyimino-N,3-dimethyl-pent-3-enamide (A.1.35), pyriminostrobin (A.1.36), bifujunzhi (A.1.37), 2-(ortho-((2,5-dimethylphenyl-oxymethylen)phenyl)-3-methoxy-acrylic acid methylester (A.1.38).

Inhibitors of complex III at Qᵢ site: cyazofamid (A.2.1), amisulbrom (A.2.2), [(6S,7R,8R)-8-benzyl-3-[(3-hydroxy-A-methoxy-pyridine-2-carbonyl)amino]-6-methyl-4,9-dioxo-1,5-dioxonan-7-yl] 2-methylpropanoate (A.2.3), fenpicoxamid (A,2.4), [(6S,7R,8R)-8-benzyl-3-[[4-methoxy-3-(propanoyloxymethoxy)pyridine-2-carbonyl]amino]-6-methyl-4,9-dioxo-1,5-dioxonan-7-yl] 2-methylpropanoate (A.2.5).

Inhibitors of complex II: benodanil (A.3.1), benzovindiflupyr (A.3.2), bixafen (A.3.3), boscalid (A.3.4), carboxin (A.3.5), fenfuram (A.3.6), fluopyram (A.3.7), flutolanil (A.3.8), fluxapyroxad (A.3.9), furametpyr (A.3.10), isofetamid (A.3.11), isopyrazam (A.3.12), mepronil (A.3.13), oxycarboxin (A.3.14), penflufen (A.3.15), penthiopyrad (A.3.16), 3-(difluoromethyl)-N-methoxy-1-methyl-N-[1-methyl-2-(2,4,6-trichlorophenyl)ethyl]pyrazole-4-carboxamide (A.3.17), N-[2-(3,4-difluorophenyl)phenyl]-3-(trifluoromethyl)pyrazine-2-carboxamide (A.3.18), sedaxane (A.3.19), tecloftalam (A.3.20), thifluzamide (A.3.21), inpyrfluxam (A.3.22), pyrapropoyne (A.3.23), fluindapyr (A.3.28), methyl (E)-2-[2-[(5-cyano-2-methyl-phenoxy)methyl]phenyl]-3-methoxy-prop-2-enoate (A.3.30), isoflucypram (A.3.31), 2-(difluoromethyl)-N-(1,1,3-trimethyl-indan-4-yl)pyridine-3-carboxamide (A.3.32), 2-(difluoromethyl)-N-[(3R)-1,1,3-trimethylindan-4-yl]pyridine-3-carboxamide (A.3.33), 2-(difluoromethyl)-N-(3-ethyl-1,1-dimethyl-indan-4-yl)¬pyridine-3-carboxamide (A.3.34), 2-(difluoromethyl)-N-[(3R)-3-ethyl-1,1-dimethyl-indan-4-yl]¬pyridine-3-carboxamide (A.3.35), 2-(difluoromethyl)-N-(1,1-dimethyl-3-propyl-indan-4-yl)¬ipy¬iridine-3-carboxamide (A.3.36), 2-(difluoromethyl)-N-[(3R)-1,1-dimethyl-3-propyl-indan-4-yl]¬pyridine-3-carboxamide (A.3.37), 2-(difluoromethyl)-N-(3-isobutyl-1,1-dimethyl-indan-4-yl)¬pyridine-3-carboxamide (A.3.38), 2-(difluoromethyl)-N-[(3R)-3-isobutyl-1,1-dimethyl-indan-4 yl]pyridine-3-carboxamide (A.3.39), 1,3-dimethyl-N-(1,1,3-trimethylindan-4-yl)pyrazole-4-carboxamide (A.3.41), 3-(difluoromethyl)-N-(7-fluoro-1,1,3-trimethyl-indan-4-yl)-1-methyl-pyrazole-4-carboxamide (A.3.42).

Other respiration inhibitors: diflumetorim (A.4.1); nitrophenyl derivates: binapacryl (A.4.2), dinobuton (A.4.3), dinocap (A.4.4), fluazinam (A.4.5), meptyldinocap (A.4.6), ferimzone (A.4.7); organometal compounds: fentin salts, e. g. fentin-acetate (A.4.8), fentin chloride (A.4.9) or fentin hydroxide (A.4.10); ametoctradin (A.4.11); silthiofam (A.4.12).

B) Sterol Biosynthesis Inhibitors (SBI Fungicides)

C14 demethylase inhibitors: triazoles: azaconazole (B.1.1), bitertanol (B.1.2), bromuconazole (B.1.3), cyproconazole (B.1.4), difenoconazole (B.1.5), diniconazole (B.1.6), diniconazole-M (B.1.7), epoxiconazole (B.1.8), fenbuconazole (B.1.9), fluquinconazole (B.1.10), flusilazole (B.1.11), flutriafol (B.1.12), hexaconazole (B.1.13), imibenconazole (B.1.14), ipconazole (B.1.15), metconazole (B.1.17), myclobutanil (B.1.18), oxpoconazole (B.1.19), paclobutrazole (B.1.20), penconazole (B.1.21), propiconazole (B.1.22), prothioconazole (B.1.23), simeconazole (B.1.24), tebuconazole (B.1.25), tetraconazole (B.1.26), triadimefon (B.1.27), triadimenol (B.1.28), triticonazole (B.1.29), uniconazole (B.1.30), 2-(2,4-difluorophenyl)-1,1-difluoro-3-(tetrazol-1-yl)-1-[5-[4-(2,2,2-trifluoroethoxy)phenyl]-2-pyridyl]propan-2-ol (B.1.31), 2-(2,4-difluorophenyl)-1,1-difluoro-3-(tetrazol-1-yl)-1-[5-[4-(trifluoromethoxy)phenyl]-2-pyridyl]propan-2-ol (B.1.32), ipfentrifluconazole (B.1.37), mefentrifluconazole (B.1.38), 2-[2-chloro-4-(4-chlorophenoxy)phenyl]-3-methyl-1-(1,2,4-triazol-1-yl)butan-2-ol (B.1.39), 2-[4-(4-chlorophenoxy)-2-(trifluoromethyl)phenyl]-1-(1,2,4-triazol-1-yl)pentan-2-ol (B.1.40), 2-[4-(4-fluorophenoxy)-2-(trifluoromethyl)phenyl]-1-(1,2,4-triazol-1-yl)propan-2-ol (B.1.41), 2-[2-chloro-4-(4-chlorophenoxy)phenyl]-1-(1,2,4-triazol-1-yl)pent-3-yn-2-ol (B.1.42), 2-(chloromethyl)-2-methyl-5-(p-tolylmethyl)-1-(1,2,4-triazol-1-ylmethyl)cyclopentanol (B.1.43); imidazoles: imazalil (B.1.44), pefurazoate (B.1.45), prochloraz (B.1.46), triflumizol (B.1.47); pyrimidines, pyridines and piperazines: fenarimol (B.1.49), pyrifenox (B.1.50), triforine (B.1.51), [3-(4-chloro-2-fluoro-phenyl)-5-(2,4-difluorophenyl)isoxazol-4-yl]-(3-pyridyl)methanol (B.1.52).

Delta14-reductase inhibitors: aldimorph (B.2.1), dodemorph (B.2.2), dodemorph-acetate (B.2.3), fenpropimorph (B.2.4), tridemorph (B.2.5), fenpropidin (B.2.6), piperalin (B.2.7), spiroxamine (B.2.8).

Inhibitors of 3-keto reductase: fenhexamid (B.3.1).

Other Sterol biosynthesis inhibitors: chlorphenomizole (B.4.1).

C) Nucleic Acid Synthesis Inhibitors

Phenylamides or acyl amino acid fungicides: benalaxyl (C.1.1), benalaxyl-M (C.1.2), kiralaxyl (C.1.3), metalaxyl (C.1.4), metalaxyl-M (C.1.5), ofurace (C.1.6), oxadixyl (C.1.7).

Other nucleic acid synthesis inhibitors: hymexazole (C.2.1), octhilinone (C.2.2), oxolinic acid (C.2.3), bupirimate (C.2.4), 5-fluorocytosine (C.2.5), 5-fluoro-2-(p-tolylmethoxy)pyrimidin-4-amine (C.2.6), 5-fluoro-2-(4-fluorophenylmethoxy)pyrimidin-4-amine (C.2.7), 5-fluoro-2-(4-chlorophenylmethoxy)pyrimidin-4 amine (C.2.8).

D) Inhibitors of Cell Division and Cytoskeleton

Tubulin inhibitors: benomyl (D.1.1), carbendazim (D.1.2), fuberidazole (D1.3), thiabendazole (D.1.4), thiophanate-methyl (D.1.5), 3-chloro-4-(2,6-difluorophenyl)-6-methyl-5-phenyl-pyrida-zine (D.1.6), 3-chloro-6-methyl-5-phenyl-4-(2,4,6-trifluorophenyl)pyridazine (D.1.7), N-ethyl-2-[(3-ethynyl-8-methyl-6-quinolyl)oxy]butanamide (D.1.8), N-ethyl-2-[(3-ethynyl-8-methyl-6-quinolyl)oxy]-2-methylsulfanyl-acetamide (D.1.9), 2-[(3-ethynyl-8-methyl-6-quinolyl)oxy]-N-(2-fluoroethyl)butanamide (D.1.10), 2-[(3-ethynyl-8-methyl-6-quinolyl)oxy]-N-(2-fluoroethyl)-2-methoxy-acetamide (D.1.11), 2-[(3-ethynyl-8-methyl-6-quinolyl)oxy]-N-propyl-butanamide (D.1.12), 2-[(3-ethynyl-8-methyl-6-quinolyl)oxy]-2-methoxy-N-propyl-acetamide (D.1.13), 2-[(3-ethynyl-8-methyl-6-quinolyl)oxy]-2-methylsulfanyl-N-propyl-acetamide (D.1.14), 2-[(3-ethynyl-8-methyl-6-quinolyl)oxy]-N-(2-fluoroethyl)-2-methylsulfanyl-acetamide (D.1.15), 4-(2-bromo-4-fluoro-phenyl)-N-(2-chloro-6-fluoro-phenyl)-2,5-dimethyl-pyrazol-3-amine (D.1.16). Other cell division inhibitors: diethofencarb (D.2.1), ethaboxam (D.2.2), pencycuron (D.2.3), fluopicolide (D.2.4), zoxamide (D.2.5), metrafenone (D.2.6), pyriofenone (D.2.7).

E) Inhibitors of Amino Acid and Protein Synthesis

Methionine synthesis inhibitors: cyprodinil (E.1.1), mepanipyrim (E.1.2), pyrimethanil (E.1.3). Protein synthesis inhibitors: blasticidin-S (E.2.1), kasugamycin (E.2.2), kasugamycin hydrochloride-hydrate (E.2.3), mildiomycin (E.2.4), streptomycin (E.2.5), oxytetracyclin (E.2.6).

F) Signal Transduction Inhibitors

MAP/histidine kinase inhibitors: fluoroimid (F.1.1), iprodione (F.1.2), procymidone (F.1.3), vinclozolin (F.1.4), fludioxonil (F.1.5). G protein inhibitors: quinoxyfen (F.2.1).

G) Lipid and Membrane Synthesis Inhibitors

Phospholipid biosynthesis inhibitors: edifenphos (G.1.1), iprobenfos (G.1.2), pyrazophos (G.1.3), isoprothiolane (G.1.4).

Lipid peroxidation: dicloran (G.2.1), quintozene (G.2.2), tecnazene (G.2.3), tolclofos-methyl (G.2.4), biphenyl (G.2.5), chloroneb (G.2.6), etridiazole (G.2.7).

Phospholipid biosynthesis and cell wall deposition: dimethomorph (G.3.1), flumorph (G.3.2), mandipropamid (G.3.3), pyrimorph (G.3.4), benthiavalicarb (G.3.5), iprovalicarb (G.3.6), valifenalate (G.3.7).

Compounds affecting cell membrane permeability and fatty acides: propamocarb (G.4.1). Inhibitors of oxysterol binding protein: oxathiapiprolin (G.5.1), 2-{3-[2-(1-{[3,5-bis(difluoromethyl-1H-pyrazol-1-yl]acetyl}piperidin-4-yl)-1,3-thiazol-4-yl]-4,5-dihydro-1,2-oxazol-5-yl}phenyl methanesulfonate (G.5.2), 2-{3-[2-(1-{[3,5-bis(difluoromethyl)-1H-pyrazol-1-yl]acetyl}piperidin-4-yl) 1,3-thiazol-4-yl]-4,5-dihydro-1,2-oxazol-5-yl}-3-chlorophenyl methanesulfonate (G.5.3), 4-[1-[2-[3-(difluoromethyl)-5-methyl-pyrazol-1-yl]acetyl]-4-piperidyl]-N-tetralin-1-yl-pyridine-2-carboxamide (G.5.4), 4-[1-[2-[3,5-bis(difluoromethyl)pyrazol-1-yl]acetyl]-4-piperidyl]-N-tetralin-1-yl-pyridine-2-carboxamide (G.5.5), 4-[1-[2-[3-(difluoromethyl)-5-(trifluoromethyl)pyrazol-1-yl]acetyl]-4-piperidyl]-N-tetralin-1-yl-pyridine-2-carboxamide (G.5.6), 4-[1-[2-[5-cyclopropyl-3-(difluoromethyl)pyrazol-1-yl]acetyl]-4-piperidyl]-N-tetralin-1-yl-pyridine-2-carboxamide (G.5.7), 4-[1-[2-[5-methyl-3-(trifluoromethyl)pyrazol-1-yl]acetyl]-4-piperidyl]-N-tetralin-1-yl-pyridine-2-carboxamide (G.5.8), 4-[1-[2-[5-(difluoromethyl)-3-(trifluoromethyl)pyrazol-1-yl]acetyl]-4-piperidyl]-N-tetralin-1-yl-pyridine-2-carboxamide (G.5.9), 4-[1-[2-[3,5-bis(trifluoromethyl)pyrazol-1-yl]acetyl]-4-piperidyl]-N-tetralin-1-yl-pyridine-2-carboxamide (G.5.10), (4-[1-[2-[5-cyclopropyl-3-(trifluoromethyl)pyrazol-1-yl]acetyl]-4-piperidyl]-N-tetralin-1-yl-pyridine-2-carboxamide (G.5.11).

H) Inhibitors with Multi Site Action

Inorganic active substances: Bordeaux mixture (H.1.1), copper (H.1.2), copper acetate (H.1.3), copper hydroxide (H.1.4), copper oxychloride (H.1.5), basic copper sulfate (H.1.6), sulfur (H.1.7).

Thio- and dithiocarbamates: ferbam (H.2.1), mancozeb (H.2.2), maneb (H.2.3), metam (H.2.4), metiram (H.2.5), propineb (H.2.6), thiram (H.2.7), zineb (H.2.8), ziram (H.2.9).

Organochlorine compounds: anilazine (H.3.1), chlorothalonil (H.3.2), captafol (H.3.3), captan (H.3.4), folpet (H.3.5), dichlofluanid (H.3.6), dichlorophen (H.3.7), hexachlorobenzene (H.3.8), pentachlorphenole (H.3.9) and its salts, phthalide (H.3.10), tolylfluanid (H.3.11).

Guanidines and others: guanidine (H.4.1), dodine (H.4.2), dodine free base (H.4.3), guazatine (H.4.4), guazatine-acetate (H.4.5), iminoctadine (H.4.6), iminoctadine-triacetate (H.4.7), iminoctadine-tris(albesilate) (H.4.8), dithianon (H.4.9), 2,6-dimethyl-1H,5H-[1,4]dithiino[2,3-c:5,6-c′]dipyrrole-1,3,5,7(2H,6H)-tetraone (H.4.10).

I) Cell Wall Synthesis Inhibitors

Inhibitors of glucan synthesis: validamycin (I.1.1), polyoxin B (I.1.2).

Melanin synthesis inhibitors: pyroquilon (I.2.1), tricyclazole (I.2.2), carpropamid (I.2.3), dicyclomet (I.2.4), fenoxanil (I.2.5).

J) Plant Defence Inducers

Acibenzolar-S-methyl (J.1.1), probenazole (J.1.2), isotianil (J.1.3), tiadinil (J.1.4), prohexadione-calcium (J.1.5); phosphonates: fosetyl (J.1.6), fosetyl-aluminum (J.1.7), phosphorous acid and its salts (J.1.8), potassium or sodium bicarbonate (J.1.9), 4-cyclopropyl-N-(2,4-dimethoxyphenyl)thiadiazole-5-carboxamide (J.1.10), calcium phosphonate (J.1.11), potassium phosphonate (J.1.12).

K) Unknown Mode of Action

Bronopol (K.1.1), chinomethionat (K.1.2), cyflufenamid (K.1.3), cymoxanil (K.1.4), dazomet (K.1.5), debacarb (K.1.6), diclocymet (K.1.7), diclomezine (K.1.8), difenzoquat (K.1.9), di-fenzoquat-methylsulfate (K.1.10), diphenylamin (K.1.11), fenitropan (K.1.12), fenpyrazamine (K.1.13), flumetover (K.1.14), flusulfamide (K.1.15), flutianil (K.1.16), harpin (K.1.17), metha-sulfocarb (K.1.18), nitrapyrin (K.1.19), nitrothal-isopropyl (K.1.20), tolprocarb (K.1.21), oxin-copper (K.1.22), proquinazid (K.1.23), tebufloquin (K.1.24), tecloftalam (K.1.25), triazoxide (K.1.26), N′-(4-(4-chloro-3-trifluoromethyl-phenoxy)-2,5-dimethyl-phenyl)-N-ethyl-N-methyl formamidine (K.1.27), N′-(4-(4-fluoro-3-trifluoromethyl-phenoxy)-2,5-dimethyl-phenyl)-N-ethyl-N-methyl formamidine (K.1.28), N′-[4-[[3-[(4-chlorophenyl)methyl]-1,2,4-thiadiazol-5-yl]oxy]-2,5-dimethyl-phenyl]-N-ethyl-N-methyl-formamidine (K.1.29), N′-(5-bromo-6-indan-2-yloxy-2-methyl-3-pyridyl)-N-ethyl-N-methyl-formamidine (K.1.30), N′-[5-bromo-6-[1-(3,5-difluorophenyl)ethoxy]-2-methyl-3-pyridyl]-N-ethyl-N-methyl-formamidine (K.1.31), N′-[5-bromo-6-(4-isopropylcyclohexoxy)-2-methyl-3-pyridyl]-N-ethyl-N-methyl-formamidine (K.1.32), N′-[5-bromo-2-methyl-6-(1-phenylethoxy)-3-pyridyl]-N-ethyl-N-methyl-formamidine (K.1.33), N′-(2-methyl-5-trifluoromethyl-4-(3-trimethylsilanyl-propoxy)-phenyl)-N-ethyl-N-methyl formamidine (K.1.34), N′-(5-difluoromethyl-2-methyl-4-(3-trimethylsilanyl-propoxy)-phenyl)-N-ethyl-N-methyl formamidine (K.1.35), 2-(4-chloro-phenyl)-N-[4-(3,4-dimethoxy-phenyl)-isoxazol-5-yl]-2-prop-2-ynyloxy-acetamide (K.1.36), 3-[5-(4-chloro-phenyl)-2,3-dimethyl-isoxazolidin-3-yl]-pyridine (pyrisoxazole) (K.1.37), 3-[5-(4-methylphenyl)-2,3-dimethyl-isoxazolidin-3 yl]-pyridine (K.1.38), 5-chloro-1-(4,6-dimethoxy-pyrimidin-2-yl)-2-methyl-1H-benzoimidazole (K.1.39), ethyl (Z)-3-amino-2-cyano-3-phenyl-prop-2-enoate (K.1.40), picarbutrazox (K.1.41), pentyl N-[6-[[(Z)-[(1-methyltetrazol-5-yl)-phenyl-methylene]amino]oxymethyl]-2-pyridyl]carbamate (K.1.42), but-3-ynyl N-[6-[[(Z)-[(1-methyltetrazol-5-yl)-phenyl-methylene]amino]oxymethyl]-2-pyridyl]carbamate (K.1.43), 2-[2-[(7,8-difluoro-2-methyl-3-quinolyl)oxy]-6-fluoro-phenyl]propan-2-ol (K.1.44), 2-[2-fluoro-6-[(8-fluoro-2-methyl-3-quinolyl)oxy]phen-yl]propan-2-ol (K.1.45), 3-(5-fluoro-3,3,4,4-tetramethyl-3,4-dihydroisoquinolin-1-yl)quinoline (K.1.46), quinofumelin (K.1.47), 3-(4,4,5-trifluoro-3,3-dimethyl-3,4-dihydroisoquinolin-1-yl)quinoline (K.1.48), 9-fluoro-2,2-dimethyl-5-(3-quinolyl)-3H-1,4-benzoxazepine (K.1.49), 2-(6-benzyl-2-pyridyl)quinazoline (K.1.50), 2-[6-(3-fluoro-4-methoxy-phenyl)-5-methyl-2-pyridyl]quinazoline (K.1.51), dichlobentiazox (K.1.52), N′-(2,5-dimethyl-4-phenoxy-phenyl)-N-ethyl-N-methyl-formamidine (K.1.53), pyrifenamine (K.1.54).

M) Growth Regulators

abscisic acid (M.1.1), amidochlor, ancymidol, 6-benzylaminopurine, brassinolide, butralin, chlormequat, chlormequat chloride, choline chloride, cyclanilide, daminozide, dikegulac, dimethipin, 2,6-dimethylpuridine, ethephon, flumetralin, flurprimidol, fluthiacet, forchlorfenuron, gibberellic acid, inabenfide, indole-3-acetic acid, maleic hydrazide, mefluidide, mepiquat, mepiquat chloride, naphthaleneacetic acid, N-6-benzyladenine, paclobutrazol, prohexadione, prohexadione-calcium, prohydrojasmon, thidiazuron, triapenthenol, tributyl phosphorotrithioate, 2,3,5-tri-iodobenzoic acid, trinexapac-ethyl and uniconazole;

N) Herbicides from Classes N.1 to N.15

N.1 Lipid biosynthesis inhibitors: alloxydim (N.1.1), alloxydim-sodium (N.1.2), butroxydim (N.1.3), clethodim (N.1.4), clodinafop (N.1.5), clodinafop-propargyl (N.1.6), cycloxydim (N.1.7), cyhalofop (N.1.8), cyhalofop-butyl (N.1.9), diclofop(N.1.10), diclofop-methyl (N.1.11), fenoxaprop (N.1.12), fenoxaprop-ethyl (N.1.13), fenoxaprop-P (N.1.14), fenoxaprop-P-ethyl (N.1.15), fluazifop (N.1.16), fluazifop-butyl (N.1.17), fluazifop-P (N.1.18), fluazifop-P-butyl (N.1.19), haloxyfop (N.1.20), haloxyfop-methyl (N.1.21), haloxyfop-P (N.1.22), haloxyfop-P-methyl (N.1.23), metamifop (N.1.24), pinoxaden (N.1.25), profoxydim (N.1.26), propaquizafop (N.1.27), quizalofop (N.1.28), quizalofop-ethyl (N.1.29), quizalofop-tefuryl (N.1.30), quizalofop-P (N.1.31), quizalofop-P-ethyl (N.1.32), quizalofop-P-tefuryl (N.1.33), sethoxydim (N.1.34), tepraloxydim (N.1.35), tralkoxydim (N.1.36), 4-(4′-chloro-4-cyclo-propyl-2′-fluoro[1,1′-biphenyl]-3-yl)-5-hydroxy-2,2,6,6-tetramethyl-2H-pyran-3(6H)-one ((N.1.37) CAS 1312337-72-6); 4-(2′,4′-dichloro-4-cyclopropyl[1,1′-biphenyl]-3-yl)-5-hydroxy-2,2,6,6-tetramethyl-2H-pyran-3(6H)-one ((N.1.38) CAS 1312337-45-3); 4-(4′-chloro-4-ethyl-2′-fluoro[1,1′-biphenyl]-3-yl)-5-hydroxy-2,2,6,6-tetramethyl-2H-pyran-3(6H)-one ((N.1.39) CAS 1033757-93-5); 4-(2′,4′-Dichloro-4-ethyl[1,1′-biphenyl]-3-yl)-2,2,6,6-tetramethyl-2H-pyran-3,5(4H,6H)-dione ((N.1.40) CAS 1312340-84-3); 5-(acetyloxy)-4-(4′-chloro-4-cyclopropyl-2′-fluoro[1,1′-biphenyl]-3-yl)-3,6-dihydro-2,2,6,6-tetramethyl-2H-pyran-3-one ((N.1.41) CAS 1312337-48-6); 5-(acetyloxy)-4-(2′,4′-dichloro-4-cyclopropyl-[1,1′-biphenyl]-3-yl)-3,6-dihydro-2,2,6,6-tetramethyl-2H-pyran-3-one (N.1.42); 5-(acetyloxy)-4-(4′-chloro-4-ethyl-2′-fluoro[1,1′-biphenyl]-3-yl)-3,6-dihydro-2,2,6,6-tetramethyl-2H-pyran-3-one ((N.1.43) CAS 1312340-82-1); 5-(acetyloxy)-4-(2′,4′-dichloro-4-ethyl[1,1′-biphenyl]-3-yl)-3,6-dihydro-2,2,6,6-tetramethyl-2H-pyran-3-one ((N.1.44) CAS 1033760-55-2); 4-(4′-chloro-4-cyclopropyl-2′-fluoro[1,1′-biphenyl]-3-yl)-5,6-dihydro-2,2,6,6-tetramethyl-5-oxo-2H-pyran-3-ylcarbonic acid methyl ester ((N.1.45) CAS 1312337-51-1); 4-(2′,4′-dichloro-4-cyclopropyl-[1,1′-biphenyl]-3-yl)-5,6-dihydro-2,2,6,6-tetramethyl-5-oxo-2H-pyran-3-yl carbonic acid methyl ester (N.1.46); 4-(4′-chloro-4-ethyl-2′-fluoro[1,1′-biphenyl]-3-yl)-5,6-dihydro-2,2,6,6-tetramethyl-5-oxo-2H-pyran-3-ylcarbonic acid methyl ester ((N.1.47) CAS 1312340-83-2); 4-(2′,4′-dichloro-4-ethyl¬[1,1′-biphenyl]-3-yl)-5,6-dihydro-2,2,6,6-tetramethyl-5-oxo-2H-pyran-3-ylcarbonic acid methyl ester ((N.1.48) CAS 1033760-58-5); benfuresate (N.1.49), butylate (N.1.50), cycloate (N.1.51), dalapon (N.1.52), dimepiperate (N.1.53), EPTC (N.1.54), esprocarb (N.1.55), ethofumesate (N.1.56), flupropanate (N.1.57), molinate (N.1.58), orbencarb (N.1.59), pebulate (N.1.60), prosulfocarb (N.1.61), TCA (N.1.62), thiobencarb (N.1.63), tiocarbazil (N.1.64), triallate (N.1.65) and vernolate (N.1.66);

N.2 ALS inhibitors: amidosulfuron (N.2.1), azimsulfuron (N.2.2), bensulfuron (N.2.3), bensul-furon-methyl (N.2.4), chlorimuron (N.2.5), chlorimuron-ethyl (N.2.6), chlorsulfuron (N.2.7), cinosulfuron (N.2.8), cyclosulfamuron (N.2.9), ethametsulfuron (N.2.10), ethametsulfuron-methyl (N.2.11), ethoxysulfuron (N.2.12), flazasulfuron (N.2.13), flucetosulfuron (N.2.14), flupyrsulfuron (N.2.15), flupyrsulfuron-methyl-sodium (N.2.16), foramsulfuron (N.2.17), halosulfuron (N.2.18), halosulfuron-methyl (N.2.19), imazosulfuron (N.2.20), iodosulfuron (N.2.21), iodosulfuron-methyl-sodium (N.2.22), iofensulfuron (N.2.23), iofensulfuron-sodium (N.2.24), mesosulfuron (N.2.25), metazosulfuron (N.2.26), metsulfuron (N.2.27), metsulfuron-methyl (N.2.28), nicosulfuron (N.2.29), orthosulfamuron (N.2.30), oxasulfuron (N.2.31), primisulfuron (N.2.32), primisulfuron-methyl (N.2.33), propyrisulfuron (N.2.34), prosulfuron (N.2.35), pyrazosulfuron (N.2.36), pyrazosulfuron-ethyl (N.2.37), rimsulfuron (N.2.38), sulfometuron (N.2.39), sulfometuron-methyl (N.2.40), sulfosulfuron (N.2.41), thifensulfuron (N.2.42), thifensulfuron-methyl (N.2.43), triasulfuron (N.2.44), tribenuron (N.2.45), tribenuron-methyl (N.2.46), trifloxysulfuron (N.2.47), triflusulfuron (N.2.48), triflusulfuron-methyl (N.2.49), tritosulfuron (N.2.50), imazamethabenz (N.2.51), imazamethabenz-methyl (N.2.52), imazamox (N.2.53), imazapic (N.2.54), imazapyr (N.2.55), imazaquin (N.2.56), imazethapyr (N.2.57); cloransulam (N.2.58), cloransulam-methyl (N.2.59), diclosulam (N.2.60), flumetsulam (N.2.61), florasulam (N.2.62), metosulam (N.2.63), penoxsulam (N.2.64), pyrimisulfan (N.2.65) and pyroxsulam (N.2.66); bispyribac (N.2.67), bispyribac-sodium (N.2.68), pyribenzoxim (N.2.69), pyriftalid (N.2.70), pyriminobac (N.2.71), pyriminobac-methyl (N.2.72), pyrithiobac (N.2.73), pyrithiobac-sodium (N.2.74), 4-[[[2-[(4,6-dimethoxy-2-pyrimidinyl)oxy]phenyl]methyl]amino]-benzoic acid-1-methyl¬ethyl ester ((N.2.75) CAS 420138-41-6), 4-[[[2-[(4,6-dimethoxy-2-pyrimidinyl)oxy]phenyl]¬methyl]amino]-benzoic acid propyl ester ((N.2.76) CAS 420138-40-5), N-(4-bromophenyl)-2-[(4,6-dimethoxy-2-pyrimidinyl)oxy]benzenemethanamine ((N.2.77) CAS 420138-01-8); flucarbazone (N.2.78), flucarbazone-sodium (N.2.79), propoxycarbazone (N.2.80), propoxycarbazone-sodium (N.2.81), thiencarbazone (N.2.82), thiencarbazone-methyl (N.2.83), triafamone (N.2.84);

N.3 Photosynthesis inhibitors: amicarbazone (N.3.1); chlorotriazine (N.3.2); ametryn (N.3.3), atrazine (N.3.4), chloridazone (N.3.5), cyanazine (N.3.6), desmetryn (N.3.7), dimethametryn (N.3.8),hexazinone (N.3.9), metribuzin (N.3.10), prometon (N.3.11), prometryn (N.3.12), propazine (N.3.13), simazine (N.3.14), simetryn (N.3.15), terbumeton (N.3.16), terbuthylazin (N.3.17), terbutryn (N.3.18), trietazin (N.3.19); chlorobromuron (N.3.20), chlorotoluron (N.3.21), chloroxuron (N.3.22), dimefuron (N.3.23), diuron (N.3.24), fluometuron (N.3.25), isoproturon (N.3.26), isouron (N.3.27), linuron (N.3.28), metamitron (N.3.29), methabenzthiazuron (N.3.30), metobenzuron (N.3.31), metoxuron (N.3.32), monolinuron (N.3.33), neburon (N.3.34), siduron (N.3.35), tebuthiuron (N.3.36), thiadiazuron (N.3.37), desmedipham (N.3.38), karbutilat (N.3.39), phenmedipham (N.3.40), phenmedipham-ethyl (N.3.41), bromofenoxim (N.3.42), bromoxynil (N.3.43) and its salts and esters, ioxynil (N.3.44) and its salts and esters, bromacil (N.3.45), lenacil (N.3.46), terbacil (N.3.47), bentazon (N.3.48), bentazon-sodium (N.3.49), pyridate (N.3.50), pyridafol (N.3.51), pentanochlor (N.3.52), propanil (N.3.53); diquat (N.3.54), diquat-dibromide (N.3.55), paraquat (N.3.56), paraquat-dichloride (N.3.57), paraquat-dimetilsulfate (N.3.58);

N.4 protoporphyrinogen-IX oxidase inhibitors: acifluorfen (N.4.1), acifluorfen-sodium (N.4.2), azafenidin (N.4.3), bencarbazone (N.4.4), benzfendizone (N.4.5), bifenox (N.4.6), butafenacil (N.4.7), carfentrazone (N.4.8), carfentrazone-ethyl (N.4.9), chlormethoxyfen (N.4.10), cinidon-ethyl (N.4.11), fluazolate (N.4.12), flufenpyr (N.4.13), flufenpyr-ethyl (N.4.14), flumiclorac (N.4.15), flumiclorac-pentyl (N.4.16), flumioxazin (N.4.17), fluoroglycofen (N.4.18), fluoroglycofen-ethyl (N.4.19), fluthiacet (N.4.20), fluthiacet-methyl (N.4.21), fomesafen (N.4.22), halosafen (N.4.23), lactofen (N.4.24), oxadiargyl (N.4.25), oxadiazon (N.4.26), oxyfluorfen (N.4.27), pentoxazone (N.4.28), profluazol (N.4.29), pyraclonil (N.4.30), pyraflufen (N.4.31), pyraflufen-ethyl (N.4.32), saflufenacil (N.4.33), sulfentrazone (N.4.34), thidiazimin (N.4.35), tiafenacil (N.4.36), trifludimoxazin (N.4.37), ethyl [3-[2-chloro-4-fluoro-5-(1-methyl-6-trifluoromethyl-2,4-dioxo-1,2,3,4-tetrahydropyrimidin-3-yl)phenoxy]-2-pyridyloxy]acetate ((N.4.38) CAS 353292-31-6), N-ethyl-3-(2,6-dichloro-4-trifluoro-methylphenoxy)-5-methyl-1H-pyrazole-1-carboxamide ((N.4.39) CAS 452098-92-9), N tetrahydrofurfuryl-3-(2,6-dichloro-4-trifluoromethylphenoxy)-5-methyl-1H-pyrazole-1-carboxamide ((N.4.40) CAS 915396-43-9), N-ethyl-3-(2-chloro-6-fluoro-4-trifluoromethyl¬phenoxy)-5-methyl-1H-pyrazole-1-carboxamide ((N.4.41) CAS 452099-05-7), N tetrahydro¬furfuryl-3-(2-chloro-6-fluoro-4-trifluoro¬methylphenoxy)-5-methyl-1H-pyrazole-1-carboxamide ((N.4.42) CAS 452100-03-7), 3-[7-fluoro-3-oxo-4-(prop-2-ynyl)-3,4-dihydro-2H-benzo[1,4]oxazin-6-yl]-1,5-dimethyl-6-thioxo-[1,3,5]triazinan-2,4-dione ((N.4.43) CAS 451484-50-7), 2-(2,2,7-trifluoro-3-oxo-4-prop-2-ynyl-3,4-dihydro-2H-benzo[1,4]oxazin-6-yl)-4,5,6,7-tetrahydro-isoindole-1,3-dione ((N.4.44) CAS 1300118-96-0), 1-methyl-6-trifluoro¬methyl-3-(2,2,7-tri-fluoro-3-oxo-4-prop-2-ynyl-3,4-dihydro-2H-benzo[1,4]oxazin-6-yl)-1H-pyrimidine-2,4-dione ((N.4.45) CAS 1304113-05-0), methyl (E)-4-[2-chloro-5-[4-chloro-5-(difluoromethoxy)-1H-methyl-pyrazol-3-yl]-4-fluoro-phenoxy]-3-methoxy-but-2-enoate ((N.4.46) CAS 948893-00-3), 3-[7-chloro-5-fluoro-2-(trifluoromethyl)-1H-benzimidazol-4-yl]-1-methyl-6-(trifluoromethyl)-1H-pyrimidine-2,4-dione ((N.4.47) CAS 212754-02-4);

N.5 Bleacher herbicides: beflubutamid (N.5.1), diflufenican (N.5.2), fluridone (N.5.3), flurochloridone (N.5.4), flurtamone (N.5.5), norflurazon (N.5.6), picolinafen (N.5.7), 4-(3-trifluoromethyl¬phenoxy)-2-(4-trifluoromethylphenyl)pyrimidine ((N.5.8) CAS 180608-33-7); benzobicyclon (N.5.9), benzofenap (N.5.10), bicyclopyrone (N.5.11), clomazone (N.5.12), fenquintrione (N.5.13), isoxaflutole (N.5.14), mesotrione (N.5.15), pyrasulfotole (N.5.16), pyrazolynate (N.5.17), pyrazoxyfen (N.5.18), sulcotrione (N.5.19), tefuryltrione (N.5.20), tembotrione (N.5.21), tolpyralate (N.5.22), topramezone (N.5.23); aclonifen (N.5.24), amitrole (N.5.25), flumeturon (N.5.26);

N.6 EPSP synthase inhibitors: glyphosate (N.6.1), glyphosate-isopropylammonium (N.6.2), glyposate-potassium (N.6.3), glyphosate-trimesium (sulfosate) (N.6.4);

N.7 Glutamine synthase inhibitors: bilanaphos (bialaphos) (N.7.1), bilanaphos-sodium (N.7.2), glufosinate (N.7.3), glufosinate-P (N.7.4), glufosinate-ammonium (N.7.5);

N.8 DHP synthase inhibitors: asulam (N.8.1);

N.9 Mitosis inhibitors: benfluralin (N.9.1), butralin (N.9.2), dinitramine (N.9.3), ethalfluralin (N.9.4), fluchloralin (N.9.5), oryzalin (N.9.6), pendimethalin (N.9.7), prodiamine (N.9.8), trifluralin (N.9.9); amiprophos (N.9.10), amiprophos-methyl (N.9.11), butamiphos (N.9.12); chlorthal (N.9.13), chlorthal-dimethyl (N.9.14), dithiopyr (N.9.15), thiazopyr (N.9.16), propyzamide (N.9.17), tebutam (N.9.18); carbetamide (N.9.19), chlorpropham (N.9.20), flamprop (N.9.21), flamprop-isopropyl (N.9.22), flamprop-methyl (N.9.23), flamprop-M-isopropyl (N.9.24), flamprop-M-methyl (N.9.25), propham (N.9.26);

N.10 VLCFA inhibitors: acetochlor (N.10.1), alachlor (N.10.2), butachlor (N.10.3), dimethachlor (N.10.4), dimethenamid (N.10.5), dimethenamid-P (N.10.6), metazachlor (N.10.7), metolachlor (N.10.8), metolachlor-S (N.10.9), pethoxamid (N.10.10), pretilachlor (N.10.11), propachlor (N.10.12), propisochlor (N.10.13), thenylchlor (N.10.14), flufenacet (N.10.15), mefenacet (N.10.16), diphenamid (N.10.17), naproanilide (N.10.18), napropamide (N.10.19), napropamide-M (N.10.20), fentrazamide (N.10.21), anilofos (N.10.22), cafenstrole (N.10.23), fenoxasulfone (N.10.24), ipfencarbazone (N.10.25), piperophos (N.10.26), pyroxasulfone (N.10.27), isoxazoline compounds of the formulae II.1, II.2, II.3, II.4, II.5, II.6, II.7, II.8 and II.9

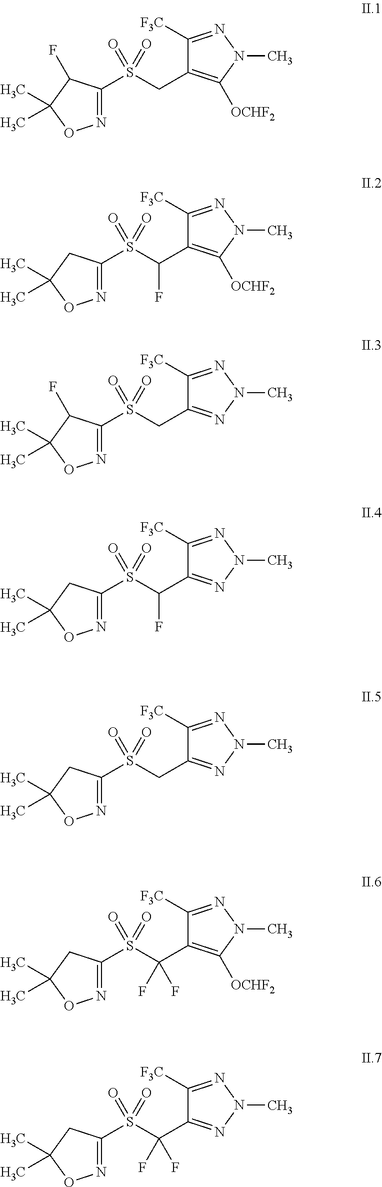

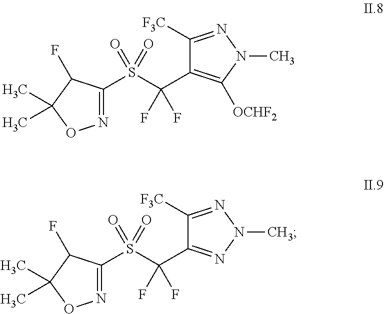

N.11 Cellulose biosynthesis inhibitors: chlorthiamid (N.11.1), dichlobenil (N.11.2), flupoxam (N.11.3), indaziflam (N.11.4), isoxaben (N.11.5), triaziflam (N.11.6), 1-cyclohexyl-5-pentafluorphenyloxy-14-[1,2,4,6]thiatriazin-3-ylamine ((N.11.7) CAS 175899-01-1); N.12 Decoupler herbicides: dinoseb (N.12.1), dinoterb (N.12.2), DNOC (N.12.3) and its salts; N.13 Auxinic herbicides: 2,4-D (N.13.1) and its salts and esters, clacyfos (N.13.2), 2,4-DB (N.13.3) and its salts and esters, aminocyclopyrachlor (N.13.4) and its salts and esters, aminopyralid (N.13.5) and its salts such as aminopyralid-dimethylammonium (N.13.6), aminopyralid-tris(2-hydroxypropyl)ammonium (N.13.7) and its esters, benazolin (N.13.8), benazolin-ethyl (N.13.9), chloramben (N.13.10) and its salts and esters, clomeprop (N.13.11), clopyralid (N.13.12) and its salts and esters, dicamba (N.13.13) and its salts and esters, dichlorprop (N.13.14) and its salts and esters, dichlorprop-P (N.13.15) and its salts and esters, fluroxypyr (N.13.16), fluroxypyr-butometyl (N.13.17), fluroxypyr-meptyl (N.13.18), halauxifen (N.13.) and its salts and esters (CAS 943832-60-8); MCPA (N.13.) and its salts and esters, MCPA-thioethyl (N.13.19), MCPB (N.13.20) and its salts and esters, mecoprop (N.13.21) and its salts and esters, mecoprop-P (N.13.22) and its salts and esters, picloram (N.13.23) and its salts and esters, quinclorac (N.13.24), quinmerac (N.13.25), TBA (2,3,6) (N.13.26) and its salts and esters, triclopyr (N.13.27) and its salts and esters, 4-amino-3-chloro-6-(4-chloro-2-fluoro-3-meth-oxyphenyl)-5-fluoropyridine-2-carboxylic acid (N.13.28), benzyl 4-amino-3-chloro-6-(4-chloro-2-fluoro-3-methoxyphenyl)-5-fluoropyridine-2-carboxylate ((N.13.29) CAS 1390661-72-9);

N.14 Auxin transport inhibitors: diflufenzopyr (N.14.1), diflufenzopyr-sodium (N.14.2), naptalam (N.14.3) and naptalam-sodium (N.14.4);

N.15 Other herbicides: bromobutide (N.15.1), chlorflurenol (N.15.2), chlorflurenol-methyl (N.15.3), cinmethylin (N.15.4), cumyluron (N.15.5), cyclopyrimorate ((N.15.6) CAS 499223-49-3) and its salts and esters, dalapon (N.15.7), dazomet (N.15.8), difenzoquat (N.15.9), difenzoquat-metilsulfate (N.15.10), dimethipin (N.15.11), DSMA (N.15.12), dymron (N.15.13), endothal (N.15.14) and its salts, etobenzanid (N.15.15), flurenol (N.15.16), flurenol-butyl (N.15.17), flurprimidol (N.15.18), fosamine (N.15.19), fosamine-ammonium (N.15.20), indanofan (N.15.21), maleic hydrazide (N.15.22), mefluidide (N.15.23), metam (N.15.24), methiozolin ((N.15.25) CAS 403640-27-7), methyl azide (N.15.26), methyl bromide (N.15.27), methyl-dymron (N.15.28), methyl iodide (N.15.29), MSMA (N.15.30), oleic acid (N.15.31), oxaziclomefone (N.15.32), pelargonic acid (N.15.33), pyributicarb (N.15.34), quinoclamine (N.15.35), tridiphane (N.15.36);

O) Insecticides from Classes 0.1 to 0.29

O.1 Acetylcholine esterase (AChE) inhibitors: aldicarb (O.1.1), alanycarb (O.1.2), bendiocarb (O.1.3), benfuracarb (O.1.4), butocarboxim (O.1.5), butoxycarboxim (O.1.6), carbaryl (O.1.7), carbofuran (O.1.8), carbosulfan (O.1.9), ethiofencarb (O.1.10), fenobucarb (O.1.11), formetanate (O.1.12), furathiocarb (O.1.13), isoprocarb (O.1.14), methiocarb (O.1.15), methomyl (O.1.16), metolcarb (O.1.17), oxamyl (O.1.18), pirimicarb (O.1.19), propoxur (O.1.20), thiodicarb (O.1.21), thiofanox (O.1.22), trimethacarb (O.1.23), XMC (O.1.24), xylylcarb (O.1.25) and triazamate (O.1.26), acephate (O.1.27), azamethiphos (O.1.28), azinphos-ethyl (O.1.29), azinphosmethyl (O.1.30), cadusafos (O.1.31), chlorethoxyfos (O.1.32), chlorfenvinphos (O.1.33), chlormephos (O.1.34), chlorpyrifos (O.1.35), chlorpyrifos-methyl (O.1.36), coumaphos (O.1.37), cyanophos (O.1.38), demeton-S-methyl (O.1.39), diazinon (O.1.40), dichlorvos/DDVP (O.1.41), dicrotophos (O.1.42), dimethoate (O.1.43), dimethylvinphos (O.1.44), disulfoton (O.1.45), EPN (O.1.46), ethion (O.1.47), ethoprophos (O.1.48), famphur (O.1.49), fenamiphos (O.1.50), fenitrothion (O.1.51), fenthion (O.1.52), fosthiazate (O.1.53), heptenophos (O.1.54), imicyafos (O.1.55), isofenphos (O.1.56), isopropyl O-(methoxyaminothio-phosphoryl) salicylate (O.1.57), isoxathion (O.1.58), malathion (O.1.59), mecarbam (O.1.60), methamidophos (O.1.61), methidathion (O.1.62), mevinphos (O.1.63), monocrotophos (O.1.64), naled (O.1.65), omethoate (O.1.66), oxydemeton-methyl (O.1.67), parathion (O.1.68), parathion-methyl (O.1.69), phenthoate (O.1.70), phorate (O.1.71), phosalone (O.1.72), phosmet (O.1.73), phosphamidon (0.1.74), phoxim (0.1.75), pirimiphos-methyl (O.1.76), profenofos (O.1.77), propetamphos (O.1.78), prothiofos (O.1.79), pyraclofos (O.1.80), pyridaphenthion (O.1.81), quinalphos (O.1.82), sulfotep (O.1.83), tebupirimfos (O.1.84), temephos (O.1.85), terbufos (O.1.86), tetrachlorvinphos (O.1.87), thiometon (O.1.88), triazophos (O.1.89), trichlorfon (O.1.90), vamidothion (O.1.91);

O.2 GABA-gated chloride channel antagonists: endosulfan (O.2.1), chlordane (O.2.2), ethiprole (O.2.3), fipronil (O.2.4), flufiprole (O.2.5), pyrafluprole (O.2.6), pyriprole (O.2.7),

O.3 Sodium channel modulators: acrinathrin (O.3.1), allethrin (O.3.2), d-cis-trans allethrin (O.3.3), d-trans allethrin (O.3.4), bifenthrin (O.3.5), bioallethrin (O.3.6), bioallethrin S-cylclopentenyl (O.3.7), bioresmethrin (O.3.8), cycloprothrin (O.3.9), cyfluthrin (O.3.10), beta-cyfluthrin (O.3.11), cyhalothrin (O.3.12), lambda-cyhalothrin (O.3.13), gamma-cyhalothrin (O.3.14), cypermethrin (O.3.15), alpha-cypermethrin (O.3.16), beta-cypermethrin (O.3.17), theta-cypermethrin (O.3.18), zeta-cypermethrin (O.3.19), cyphenothrin (O.3.20), deltamethrin (O.3.21), empenthrin (O.3.22), esfenvalerate (O.3.23), etofenprox (O.3.24), fenpropathrin (O.3.25), fenvalerate (O.3.26), flucythrinate (O.3.27), flumethrin (O.3.28), tau-fluvalinate (O.3.29), halfenprox (O.3.30), heptafluthrin (O.3.31), imiprothrin (O.3.32), meperfluthrin (O.3.33), metofluthrin (O.3.34), momfluorothrin (O.3.35), permethrin (O.3.36), phenothrin (O.3.37), prallethrin (O.3.38), profluthrin (O.3.39), pyrethrin (pyrethrum) (O.3.40), resmethrin (O.3.41), silafluofen (O.3.42), tefluthrin (O.3.43), tetramethylfluthrin (O.3.44), tetramethrin (O.3.45), tralomethrin (O.3.46) and transfluthrin (O.3.47), DDT (O.3.48), methoxychlor (O.3.49);

O.4 Nicotinic acetylcholine receptor agonists (nAChR): acetamiprid (O.4.1), clothianidin (O.4.2), cycloxaprid (O.4.3), dinotefuran (O.4.4), imidacloprid (O.4.5), nitenpyram (O.4.6), thiacloprid (O.4.7), thiamethoxam (O.4.8), (2E)-1-[(6-chloropyridin-3-yl)methyl]-N′-nitro-2-pentylidene-hydrazinecarboximidamide (O.4.9), 1-[(6-chloropyridin-3-yl)methyl]-7-methyl-8-nitro-5-propoxy-1,2,3,5,6,7-hexahydroimidazo[1,2-a]pyridine (O.4.10), nicotine (O.4.11), sulfoxaflor (O.4.12), flupyradifurone (O.4.13), triflumezopyrim (O.4.14);

O.5 Nicotinic acetylcholine receptor allosteric activators: spinosad (O.5.1), spinetoram (O.5.2);

O.6 Chloride channel activators: abamectin (O.6.1), emamectin benzoate (O.6.2), ivermectin (O.6.3), lepimectin (O.6.4), milbemectin (O.6.5);

O.7 Juvenile hormone mimics: hydroprene (O.7.1), kinoprene (O.7.2), methoprene (O.7.3), fenoxycarb (O.7.4), pyriproxyfen (O.7.5);

O.8 miscellaneous non-specific (multi-site) inhibitors: methyl bromide (O.8.1) and other alkyl halides, chloropicrin (O.8.2), sulfuryl fluoride (O.8.3), borax (O.8.4), tartar emetic (O.8.5);

O.9 Chordotonal organ TRPV channel modulators: pymetrozine (O.9.1), flonicamid (O.9.2), pyrifluquinazon (O.9.3);

O.10 Mite growth inhibitors: clofentezine (O.10.1), hexythiazox (O.10.2), diflovidazin (O.10.3), etoxazole (O.10.4);

O.11 Microbial disruptors of insect midgut membranes: the Bt crop proteins: Cry1Ab, Cry1Ac, Cry1Fa, Cry2Ab, mCry3A, Cry3Ab, Cry3Bb, Cry34/35Ab1;

O.12 Inhibitors of mitochondrial ATP synthase: diafenthiuron (O.12.1), azocyclotin (O.12.2), cyhexatin (O.12.3), fenbutatin oxide (O.12.4), propargite (O.12.5), tetradifon (O.12.6);

O.13 Uncouplers of oxidative phosphorylation via disruption of the proton gradient: chlorfenapyr (O.13.1), DNOC (O.13.2), sulfluramid (O.13.3);

O.14 Nicotinic acetylcholine receptor (nAChR) channel blockers: bensultap (O.14.1), cartap hydrochloride (O.14.2), thiocyclam (O.14.3), thiosultap sodium (O.14.4);

O.15 Inhibitors of the chitin biosynthesis type 0: bistrifluron (O.15.1), chlorfluazuron (O.15.2), diflubenzuron (O.15.3), flucycloxuron (O.15.4), flufenoxuron (O.15.5), hexaflumuron (O.15.6), lufenuron (O.15.7), novaluron (O.15.8), noviflumuron (O.15.9), teflubenzuron (O.15.10), triflumuron (O.15.11);

O.16 Inhibitors of the chitin biosynthesis type 1: buprofezin (O.16.1);

O.17 Moulting disruptors: cyromazine (O.17.1);

O.18 Ecdyson receptor agonists: methoxyfenozide (O.18.1), tebufenozide (O.18.2), halofenozide (O.18.3), fufenozide (O.18.4), chromafenozide (O.18.5);

O.19 Octopamin receptor agonists: amitraz (O.19.1);

O.20 Mitochondrial complex III electron transport inhibitors: hydramethylnon (O.20.1), acequinocyl (O.20.2), fluacrypyrim (O.20.3), bifenazate (O.20.4);

O.21 Mitochondrial complex I electron transport inhibitors: fenazaquin (O.21.1), fenpyroximate (O.21.2), pyrimidifen (O.21.3), pyridaben (O.21.4), tebufenpyrad (O.21.5), tolfenpyrad (O.21.6), rotenone (O.21.7);

O.22 Voltage-dependent sodium channel blockers: indoxacarb (O.22.1), metaflumizone (O.22.2), 2-[2-(4-cyanophenyl)-1-[3-(trifluoromethyl)phenyl]ethylidene]-N-[4-(difluoromethoxy)phenyl]-hydrazinecarboxamide (O.22.3), N-(3-chloro-2-methylphenyl)-2-[(4-chlorophenyl)-[4-[methyl(methylsulfonyl)amino]phenyl]methylene]-hydrazinecarboxamide (O.22.4);

O.23 Inhibitors of the of acetyl CoA carboxylase: spirodiclofen (O.23.1), spiromesifen (O.23.2), spirotetramat (O.23.3), spiropidion (O.23.4);

O.24 Mitochondrial complex IV electron transport inhibitors: aluminium phosphide (O.24.1), calcium phosphide (O.24.2), phosphine (O.24.3), zinc phosphide (O.24.4), cyanide (O.24.5);

O.25 Mitochondrial complex II electron transport inhibitors: cyenopyrafen (O.25.1), cyflumetofen (O.25.2);

O.26 Ryanodine receptor-modulators: flubendiamide (O.26.1), chlorantraniliprole (O.26.2), cyantraniliprole (O.26.3), cyclaniliprole (O.26.4), tetraniliprole (O.26.5), (R)-3-chloro-N1-{2-methyl-4-[1,2,2,2-tetrafluoro-1-(trifluoromethyl)ethyl]phenyl}-N2-(1-methyl-2-methylsulfonylethyl)phthalamide (O.26.6), (S)-3-chloro-N1-{2-methyl-4-[1,2,2,2-tetrafluoro-1-(trifluoromethyl)ethyl]phenyl}-N2-(1-methyl-2-methylsulfonylethyl)phthalamide (O.26.7), methyl-2-[3,5-dibromo-2-({[3-bromo-1-(3-chloropyridin-2-yl)-1H-pyrazol-5-yl]carbonyl}amino)benzoyl]-1,2-dimethylhydrazinecarboxylate (0.26.8), N-[4,6-dichloro-2-[(diethyl-lambda-4-sulfanylidene)-carbamoyI]-phenyl]-2-(3-chloro-2-pyridyl)-5-(trifluoromethyl)pyrazole-3-carboxamide (O.26.9), N-[4-chloro-2-[(diethyl-lambda-4-sulfanylidene)carbamoyl]-6-methyl-phenyl]-2-(3-chloro-2-pyridyl)-5-(trifluoromethyl)pyrazole-3-carboxamide (O.26.10), N-[4-chloro-2-[(di-2-propyl-lambda-4-sulfanylidene)carbamoyl]-6-methyl-phenyl]-2-(3-chloro-2-pyridyl)-5-(trifluoromethyl)pyrazole-3-carboxamide (O.26.11), N-[4,6-dichloro-2-[(di-2-propyl-lambda-4-sulfanylidene)carbamoyl]-phenyl]-2-(3-chloro-2-pyridyl)-5-(trifluoromethyl)pyrazole-3-carboxamide (O.26.12), N-[4,6-di-bromo-2-[(diethyl-lambda-4-sulfanylidene)carbamoyl]-phenyl]-2-(3-chloro-2-pyridyl)-5-(tri-fluoromethyl)pyrazole-3-carboxamide (O.26.13), N-[2-(5-amino-1,3,4-thiadiazol-2-yl)-4-chloro-6-methylphenyl]-3-bromo-1-(3-chloro-2-pyridinyl)-1H-pyrazole-5-carboxamide (O.26.14), 3-chloro-1-(3-chloro-2-pyridinyl)-N-[2,4-dichloro-6-[[(1-cyano-1-methylethyl)amino]carbonyl]phenyl]-1H-pyrazole-5-carboxamide (O.26.15), 3-bromo-N-[2,4-dichloro-6-(methylcarbamoyl)phenyl]-1-(3,5-dichloro-2-pyridyl)-1H-pyrazole-5-carboxamide (O.26.16), N-[4-chloro-2-[[(1,1-dimethylethyl)amino]carbonyl]-6-methylphenyl]-1-(3-chloro-2-pyridinyl)-3-(fluoromethoxy)-1H-pyrazole-5-carboxamide (O.26.17), cyhalodiamide (O.26.18);

O.27. insecticidal active compounds of unknown or uncertain mode of action: afidopyropen (O.27.1), afoxolaner (O.27.2), azadirachtin (O.27.3), amidoflumet (O.27.4), benzoximate (O.27.5), broflanilide (O.27.7), bromopropylate (O.27.8), chinomethionat (O.27.9), cryolite (O.27.10), dicloromezotiaz (O.27.11), dicofol (O.27.12), flufenerim (O.27.13), flometoquin (O.27.14), fluensulfone (O.27.15), fluhexafon (O.27.16), fluopyram (O.27.17), fluralaner (O.27.19), metoxadiazone (O.27.20), piperonyl butoxide (O.27.21), pyflubumide (O.27.22), pyridalyl (O.27.23), tioxazafen (O.27.26), 11-(4-chloro-2,6-dimethylphenyl)-12-hydroxy-1,4-dioxa-9-azadispiro[4.2.4.2]-tetradec-11-en-10-one (O.27.28), 3-(4′-fluoro-2,4-dimethylbiphenyl-3-yl)-4-hydroxy-8-oxa-1-azaspiro[4.5]dec-3-en-2-one (O.27.28), 1-[2-fluoro-4-methyl-5-[(2,2,2-trifluoroethyl)sulfinyl]phenyl]-3-(trifluoromethyl)-1H-1,2,4-triazole-5-amine (O.27.29), Bacillus firmus I-1582 (O.27.30), flupyrimin (O.27.31), fluazaindolizine (O.27.42), 4-[5-(3,5-dichlorophenyl)-5-(trifluoromethyl)-4H-isoxazol-3-yl]-2-methyl-N-(1-oxothietan-3-yl)benzamide (O.27.43), fluxametamide (O.27.44), 5-[3-[2,6-dichloro-4-(3,3-dichloroallyloxy)phenoxy]propoxy]-1H-pyrazole (O.27.45), 3-(benzoylmethylamino)-N-[2-bromo-4-[1,2,2,3,3,3-hexafluoro-1-(trifluoromethyl)propyl]-6-(trifluoromethyl)phenyl]-2-fluoro-benzamide (O.27.46), 3-(benzoylmethylamino)-2-fluoro-N-[2-iodo-4-[1,2,2,2-tetrafluoro-1-(trifluoromethyl)ethyl]-6-(trifluoromethyl)phenyl]-benzamide (O.27.47), N-[3-[[[2-iodo-4-[1,2,2,2-tetrafluoro-1-(trifluoromethyl)ethyl]-6-(trifluoromethyl)phenyl]amino]carbonyl]phenyl]-N-methyl-benzamide (O.27.48), N-[3-[[[2-bromo-4-[1,2,2,2-tetrafluoro-1-(trifluoromethyl)ethyl]-6-(trifluoromethyl)phenyl]amino]carbonyl]-2-fluorophenyl]-4-fluoro-N-methyl-benzamide (O.27.49), 4-fluoro-N-[2-fluoro-3-[[[2-iodo-4-[1,2,2,2-tetrafluoro-1-(trifluoromethyl)ethyl]-6-(trifluoro-methyl)phenyl]amino]carbonyl]phenyl]-N-methyl-benzamide (O.27.50), 3-fluoro-N-[2-fluoro-3-[[[2-iodo-4-[1,2,2,2-tetrafluoro-1-(trifluoromethyl)ethyl]-6(trifluoromethyl)phenyl]amino]-carbonyl]phenyl]-N-methyl-benzamide (O.27.51), 2-chloro-N-[3-[[[2-iodo-4-[1,2,2,2-tetrafluoro-1-(trifluoromethyl)ethyl]-6-(trifluoromethyl)phenyl]amino]carbonyl]phenyl]-3-pyridinecarboxamide (O.27.52), 4-cyano-N-[2-cyano-5-[[2,6-dibromo-4-[1,2,2,3,3,3-hexafluoro-1-(trifluoromethyl)propyl]phenyl]carbamoyl]phenyl]-2-methyl-benzamide (O.27.53), 4-cyano-3-[(4-cyano-2-methyl-benzoyl)amino]-N-[2,6-dichloro-4-[1,2,2,3,3,3-hexafluoro-1-(trifluoromethyl)-propyl]phenyl]-2-fluoro-benzamide (O.27.54), N-[5-[[2-chloro-6-cyano-4-[1,2,2,3,3,3-hexafluoro-1-(trifluoromethyl)propyl]phenyl]carbamoyl]-2-cyano-phenyl]-4-cyano-2-methyl-benzamide (O.27.55), N-[5-[[2-bromo-6-chloro-4-[2,2,2-trifluoro-1-hydroxy-1-(trifluoromethyl)ethyl]phenyl]carbamoyl]-2-cyano-phenyl]-4-cyano-2-methyl-benzamide (O.27.56), N-[5-[[2-bromo-6-chloro-4-[1,2,2,3,3,3-hexafluoro-1-(trifluoromethyl)-propyl]phenyl]carbamoyl]-2-cyano-phenyl]-4-cyano-2-methyl-benzamide (O.27.57), 4-cyano-N-[2-cyano-5-[[2,6-dichloro-4-[1,2,2,3,3,3-hexafluoro-1-(trifluoromethyl)propyl]phenyl]-carbamoyl]phenyl]-2-methyl-benzamide (O.27.58), 4-cyano-N-[2-cyano-5-[[2,6-dichloro-4-[1,2,2,2-tetrafluoro-1-(trifluoromethyl)ethyl]phenyl]carbamoyl]phenyl]-2-methyl-benzamide (O.27.59), N-[5-[[2-bromo-6-chloro-4-[1,2,2,2-tetrafluoro-1-(trifluoromethyl)ethyl]phenyl]carbamoyl]-2-cyano-phenyl]-4-cyano-2-methyl-benzamide (O.27.60), 2-(1,3-dioxan-2-yl)-6-[2-(3-pyridinyl)-5-thiazolyl]-pyridine, 2-[6-[2-(5-fluoro-3-pyridinyl)-5-thiazolyl]-2-pyridinyl]-pyrimidine (O.27.61), 2-[6-[2-(3-pyridinyl)-5-thiazolyl]-2-pyridinyl]-pyrimidine (O.27.62), N-methylsulfonyl-6-[2-(3-pyridyl)thiazol-5-yl]pyridine-2-car-boxamide (O.27.63), N-methylsulfonyl-6-[2-(3-pyridyl)thiazol-5-yl]pyridine-2-carboxamide (O.27.64), N-ethyl-N-[4-methyl-2-(3-pyridyl)thiazol-5-yl]-3-methylthio-propanamide (O.27.65), N-methyl-N-[4-methyl-2-(3-pyridyl)thiazol-5-yl]-3-methylthio-propanamide (O.27.66), N,2-dimethyl-N-[4-methyl-2-(3-pyridyl)thiazol-5-yl]-3-methylthio-propanamide (O.27.67), N-ethyl-2-methyl-N-[4-methyl-2-(3-pyridyl)thiazol-5-yl]-3-methylthio-propanamide (O.27.68), N-[4-chloro-2-(3-pyridyl)thiazol-5-yl]-N-ethyl-2-methyl-3-methylthio-propanamide (O.2769.), N-[4-chloro-2-(3-pyridyl)thiazol-5-yl]-N,2-dimethyl-3-methylthio-propanamide (O.27.70), N-[4-chloro-2-(3-pyridyl)thiazol-5-yl]-N-methyl-3-methylthio-propanamide (O.27.71), N-[4-chloro-2-(3-pyridyl)thiazol-5-yl]-N-ethyl-3-methylthio-propanamide (O.27.72), 1-[(6-chloro-3-pyridinyl)methyl]-1,2,3,5,6,7-hexahydro-5-methoxy-7-methyl-8-nitro-imidazo[1,2-a]pyridine (O.27.73), 1-[(6-chloropyridin-3-yl)methyl]-7-methyl-8-nitro-1,2,3,5,6,7-hexahydroimidazo[1,2-a]pyridin-5-ol (O.27.74), 1-isopropyl-N,5-dimethyl-N-pyridazin-4-yl-pyrazole-4-carboxamide (O.27.75), 1-(1,2-dimethylpropyl)-N-ethyl-5-methyl-N-pyridazin-4-yl-pyrazole-4-carboxamide (O.27.76), N,5-dimethyl-N-pyridazin-4-yl-1-(2,2,2-trifluoro-1-methyl-ethyl)pyrazole-4-carboxamide (O.27.77), 1-[1-(1-cyanocyclopropyl)ethyl]-N-ethyl-5-methyl-N-pyridazin-4-yl-pyrazole-4-carboxamide (O.27.78), N-ethyl-1-(2-fluoro-1-methyl-propyl)-5-methyl-N-pyridazin-4-yl-pyrazole-4-carboxamide (O.27.79), 1-(1,2-dimethylpropyl)-N,5-dimethyl-N-pyridazin-4-yl-pyrazole-4-carboxamide (O.27.80), 1-[1-(1-cyanocyclopropyl)ethyl]-N,5-dimethyl-N-pyridazin-4-yl-pyrazole-4-carboxamide (O.27.81), N-methyl-1-(2-fluoro-1-methyl-propyl]-5-methyl-N-pyridazin-4-yl-pyrazole-4-carboxamide (O.27.82), 1-(4,4-difluorocyclohexyl)-N-ethyl-5-methyl-N-pyridazin-4-yl-pyrazole-4-carboxamide (O.27.83), 1-(4,4-difluorocyclohexyl)-N,5-dimethyl-N-pyridazin-4-yl-pyrazole-4-carboxamide (O.27.84), N-(1-methylethyl)-2-(3-pyridinyl)-2H-indazole-4-carboxamide (O.27.85), N-cyclopropyl-2-(3-pyridinyl)-2H-indazole-4-carboxamide (O.27.86), N-cyclohexyl-2-(3-pyridinyl)-2H-indazole-4-carboxamide (O.27.87), 2-(3-pyridinyl)-N-(2,2,2-trifluoroethyl)-2H-indazole-4-carboxamide (O.27.88), 2-(3-pyridinyl)-N-[(tetrahydro-2-furanyl)methyl]-2H-indazole-5-carboxamide (O.27.89), methyl 2-[[2-(3-pyridinyl)-2H-indazol-5-yl]carbonyl]hydrazinecarboxylate (O.27.90), N-[(2,2-difluorocyclopropyl)methyl]-2-(3-pyridinyl)-2H-indazole-5-carboxamide (O.27.91), N-(2,2-difluoropropyl)-2-(3-pyridinyl)-2H-indazole-5-carboxamide (O.27.92), 2-(3-pyridinyl)-N-(2-pyrimidinylmethyl)-2H-indazole-5-carboxamide (O.27.93), N-[(5-methyl-2-pyrazinyl)methyl]-2-(3-pyridinyl)-2H-indazole-5-carboxamide (O.27.94), N-[3-chloro-1-(3-pyridyl)pyrazol-4-yl]-N-ethyl-3-(3,3,3-trifluoropropylsulfanyl)-propanamide (O.27.95), tyclopyrazoflor (O.27.96), N-[3-chloro-1-(3-pyridyl)pyrazol-4-yl]-3-[(2,2-difluorocyclopropyl)methylsulfanyl]-N-ethyl-propanamide (O.27.97), N-[3-chloro-1-(3-pyridyl)pyrazol-4-yl]-3-[(2,2-difluorocyclopropyl)methylsulfinyl]-N-ethyl-propanamide (O.27.98), sarolaner (O.27.99), lotilaner (O.27.100), N-[4-chloro-3-[[(phenylmethyl)amino]carbonyl]phenyl]-1-methyl-3-(1,1,2,2,2-pentafluoroethyl)-4-(trifluoromethyl)-1H-pyrazole-5-carboxamide (O.27.101), M.UN.22a 2-(3-ethylsulfonyl-2-pyridyl)-3-methyl-6-(trifluoromethyl)imidazo[4,5-b]pyridine (O.27.102), 2-[3-ethylsulfonyl-5-(trifluoromethyl)-2-pyridyl]-3-methyl-6-(trifluoromethyl)imidazo[4,5-b]pyridine (O.27.103), 4-[5-(3,5-dichlorophenyl)-5-(trifluoromethyl)-4H-isoxazol-3-yl]-N-[(4R)-2-ethyl-3-oxo-isoxazolidin-4-yl]-2-methyl-benzamide (O.27.104), 4-[5-(3,5-dichloro-4-fluoro-phenyl)-5-(trifluoromethyl)-4H-isoxazol-3-yl]-N-[(4R)-2-ethyl-3-oxo-isoxazolidin-4-yl]-2-methyl-benzamide (O.27.105), N-[4-chloro-3-(cyclopropylcarbamoyl)phenyl]-2-methyl-5-(1,1,2,2,2-pentafluoroethyl)-4-(trifluoromethyl)pyrazole-3-carboxamide (O.27.106), N-[4-chloro-3-[(1-cyanocyclopropyl)carbamoyl]phenyl]-2-methyl-5-(1,1,2,2,2-pentafluoroethyl)-4-(trifluoromethyl)pyrazole-3-carboxamide (O.27.107), acynonapyr (O.27.108), benzpyrimoxan (O.27.109), chloro-N-(1-cyanocyclopropyl)-5-[1-[2-methyl-5-(1,1,2,2,2-pentafluoroethyl)-4-(trifluoromethyl)pyrazol-3-yl]pyrazol-4-yl]benzamide (O.27.110).

The active substances referred to as component 2, their preparation and their activity e. g. against harmful fungi is known (cf.: http://www.alanwood.net/pesticides/); these substances are commercially available. The compounds described by IUPAC nomenclature, their preparation and their pesticidal activity are also known (cf. Can. J. Plant Sci. 48(6), 587-94, 1968; EP-A 141 317; EP-A 152 031; EP-A 226 917; EP-A 243 970; EP-A 256 503; EP-A 428 941; EP-A 532 022; EP-A 1 028 125; EP-A 1 035 122; EP-A 1 201 648; EP-A 1 122 244, JP 2002316902; DE 19650197; DE 10021412; DE 102005009458; U.S. Pat. No. 3,296,272; U.S. Pat. No. 3,325,503; WO 98/46608; WO 99/14187; WO 99/24413; WO 99/27783; WO 00/29404; WO 00/46148; WO 00/65913; WO 01/54501; WO 01/56358; WO 02/22583; WO 02/40431; WO 03/10149; WO 03/11853; WO 03/14103; WO 03/16286; WO 03/53145; WO 03/61388; WO 03/66609; WO 03/74491; WO 04/49804; WO 04/83193; WO 05/120234; WO 05/123689; WO 05/123690; WO 05/63721; WO 05/87772; WO 05/87773; WO 06/15866; WO 06/87325; WO 06/87343; WO 07/82098; WO 07/90624, WO 10/139271, WO 11/028657, WO 12/168188, WO 07/006670, WO 11/77514; WO 13/047749, WO 10/069882, WO 13/047441, WO 03/16303, WO 09/90181, WO 13/007767, WO 13/010862, WO 13/127704, WO 13/024009, WO 13/24010, WO 13/047441, WO 13/162072, WO 13/092224, WO 11/135833, CN 1907024, CN 1456054, CN 103387541, CN 1309897, WO 12/84812, CN 1907024, WO 09094442, WO 14/60177, WO 13/116251, WO 08/013622, WO 15/65922, WO 94/01546, EP 2865265, WO 07/129454, WO 12/165511, WO 11/081174, WO 13/47441, JP2015089883, JP2015120675, WO2015119246, WO2011135827, WO2012084812).

The present invention furthermore relates to agrochemical compositions comprising a mixture of at least one compound I (component 1) and at least one further active substance useful for plant protection, e. g. selected from the groups A) to O) (component 2), in particular one further fungicide, e. g. one or more fungicide from the groups A) to K), as described above, and if desired one suitable solvent or solid carrier. Those mixtures are of particular interest, since many of them at the same application rate show higher efficiencies against harmful fungi. Furthermore, combating harmful fungi with a mixture of compounds I and at least one fungicide from groups A) to K), as described above, is more efficient than combating those fungi with individual compounds I or individual fungicides from groups A) to K).

By applying compounds I together with at least one active substance from groups A) to O) a synergistic effect can be obtained, i.e. more then simple addition of the individual effects is obtained (synergistic mixtures).

This can be obtained by applying the compounds I and at least one further active substance simultaneously, either jointly (e. g. as tank-mix) or seperately, or in succession, wherein the time interval between the individual applications is selected to ensure that the active substance applied first still occurs at the site of action in a sufficient amount at the time of application of the further active substance(s). The order of application is not essential for working of the present invention.

When applying compound I and a pesticide II sequentially the time between both applications may vary e. g. between 2 hours to 7 days. Also a broader range is possible ranging from 0.25 hour to 30 days, preferably from 0.5 hour to 14 days, particularly from 1 hour to 7 days or from 1.5 hours to 5 days, even more preferred from 2 hours to 1 day.

In the binary mixtures and compositions according to the invention the weight ratio of the component 1) and the component 2) generally depends from the properties of the active components used, usually it is in the range of from 1:10,000 to 10,000:1, often it is in the range of from 1:100 to 100:1, regularly in the range of from 1:50 to 50:1, preferably in the range of from 1:20 to 20:1, more preferably in the range of from 1:10 to 10:1, even more preferably in the range of from 1:4 to 4:1 and in particular in the range of from 1:2 to 2:1.

According to further embodiments of the binary mixtures and compositions, the weight ratio of the component 1) and the component 2) usually is in the range of from 1000:1 to 1:1, often in the range of from 100:1 to 1:1, regularly in the range of from 50:1 to 1:1, preferably in the range of from 20:1 to 1:1, more preferably in the range of from 10:1 to 1:1, even more preferably in the range of from 4:1 to 1:1 and in particular in the range of from 2:1 to 1:1.

According to a further embodiments of the binary mixtures and compositions, the weight ratio of the component 1) and the component 2) usually is in the range of from 1:1 to 1:1000, often in the range of from 1:1 to 1:100, regularly in the range of from 1:1 to 1:50, preferably in the range of from 1:1 to 1:20, more preferably in the range of from 1:1 to 1:10, even more preferably in the range of from 1:1 to 1:4 and in particular in the range of from 1:1 to 1:2.

In the ternary mixtures, i.e. compositions according to the invention comprising the component 1) and component 2) and a compound III (component 3), the weight ratio of component 1) and component 2) depends from the properties of the active substances used, usually it is in the range of from 1:100 to 100:1, regularly in the range of from 1:50 to 50:1, preferably in the range of from 1:20 to 20:1, more preferably in the range of from 1:10 to 10:1 and in particular in the range of from 1:4 to 4:1, and the weight ratio of component 1) and component 3) usually it is in the range of from 1:100 to 100:1, regularly in the range of from 1:50 to 50:1, preferably in the range of from 1:20 to 20:1, more preferably in the range of from 1:10 to 10:1 and in particular in the range of from 1:4 to 4:1.

Any further active components are, if desired, added in a ratio of from 20:1 to 1:20 to the component 1).

These ratios are also suitable for inventive mixtures applied by seed treatment.

Accordingly, the present invention furthermore relates to mixtures comprising one compound of the formula I (component 1) and one pesticide II (component 2), wherein pesticide II is an active ingredient selected from the groups A) to O) defined above.

Further embodiments B-1 to B-1128 listed in Table B below relate to the mixtures comprising as active components one of the in the present specification individualized compounds of the formula I, which is selected from the group of compounds I.A.A-1 to I.A.A-323, I.B.A-1 to I.B.A-323, I.C.A-1 to I.C.A-323, I.D.A-1 to I.D.A-323, I.E.A-1 to I.E.A-323, I.F.A-1 to I.F.A-323, I.G.A-1 to I.G.A-323, I.H.A-1 to I.H.A-323, I.J.A-1 to I.J.A-323, I.K.A-1 to I.K.A-323 and I.L.A-1 to I.L.A-323 as defined in tables 1 to 11 above (component 1, a group represented by the expression “(I)”) and one pesticide II selected from the groups A) to O) as defined herein (component 2). Further embodiments B-1 to B-1128 listed in Table B below relate to the mixtures comprising as active components one of the in the present specification individualized compounds of the formula I, which is selected from the group of compounds Ex-1 to Ex-15 as defined in table I below (component 1, a group represented by the expression “(I)”) and one pesticide II selected from the groups A) to O) as defined herein (component 2).

Preferably, the compositions described in Table B comprise the active components in synergistically effective amounts.

TABLE B
B-1: (I) + (A.1.1), B-2: (I) + (A.1.2), B-3: (I) + (A.1.3), B-4: (I) + (A.1.4), B-5: (I) + (A.1.5),
B-6: (I) + (A.1.6), B-7: (I) + (A.1.7), B-8: (I) + (A.1.8), B-9: (I) + (A.1.9), B-10: (I) + (A.1.10),
B-11: (I) + (A.1.11), B-12: (I) + (A.1.12), B-13: (I) + (A.1.13), B-14: (I) + (A.1.14), B-15: (I) + (A.1.15),
B-16: (I) + (A.1.16), B-17: (I) + (A.1.17), B-18: (I) + (A.1.18), B-19: (I) + (A.1.19), B-20: (I) + (A.1.20),
B-21: (I) + (A.1.21), B-22: (I) + (A.1.22), B-23: (I) + (A.1.25), B-24: (I) + (A.1.33), B-25: (I) + (A.1.34),
B-26: (I) + (A.1.35), B-27: (I) + (A.1.36), B-28: (I) + (A.1.37), B-29: (I) + (A.1.38), B-30: (I) + (A.2.1),
B-31: (I) + (A.2.2), B-32: (I) + (A.2.3), B-33: (I) + (A.2.4), B-34: (I) + (A.2.5), B-35: (I) + (A.3.1),
B-36: (I) + (A.3.2), B-37: (I) + (A.3.3), B-38: (I) + (A.3.4), B-39: (I) + (A.3.5), B-40: (I) + (A.3.6),
B-41: (I) + (A.3.7), B-42: (I) + (A.3.8), B-43: (I) + (A.3.9), B-44: (I) + (A.3.10), B-45: (I) + (A.3.11),
B-46: (I) + (A.3.12), B-47: (I) + (A.3.13), B-48: (I) + (A.3.14), B-49: (I) + (A.3.15), B-50: (I) + (A.3.16),
B-51: (I) + (A.3.17), B-52: (I) + (A.3.18), B-53: (I) + (A.3.19), B-54: (I) + (A.3.20), B-55: (I) + (A.3.21),
B-56: (I) + (A.3.22), B-57: (I) + (A.3.23), B-58: (I) + (A.3.24), B-59: (I) + (A.3.25), B-60: (I) + (A.3.26),
B-61: (I) + (A.3.27), B-62: (I) + (A.3.28), B-63: (I) + (A.3.30), B-64: (I) + (A.3.31), B-65: (I) + (A.3.32),
B-66: (I) + (A.3.33), B-67: (I) + (A.3.34), B-68: (I) + (A.3.35), B-69: (I) + (A.3.36), B-70: (I) + (A.3.37),
B-71: (I) + (A.3.38), B-72: (I) + (A.3.39), B-73: (I) + (A.3.40), B-74: (I) + (A.3.41), B-75: (I) + (A.3.42),
B-76: (I) + (A.4.1), B-77: (I) + (A.4.2), B-78: (I) + (A.4.3), B-79: (I) + (A.4.4), B-80: (I) + (A.4.5),
B-81: (I) + (A.4.6), B-82: (I) + (A.4.7), B-83: (I) + (A.4.8), B-84: (I) + (A.4.9), B-85: (I) + (A.4.10),
B-86: (I) + (A.4.11), B-87: (I) + (A.4.12), B-88: (I) + (B.1.1), B-89: (I) + (B.1.2), B-90: (I) + (B.1.3),
B-91: (I) + (B.1.4), B-92: (I) + (B.1.5), B-93: (I) + (B.1.6), B-94: (I) + (B.1.7), B-95: (I) + (B.1.8),
B-96: (I) + (B.1.9), B-97: (I) + (B.1.10), B-98: (I) + (B.1.11), B-99: (I) + (B.1.12), B-100: (I) + (B.1.13),
B-101: (I) + (B.1.14), B-102: (I) + (B.1.15), B-103: (I) + (B.1.16), B-104: (I) + (B.1.17),
B-105: (I) + (B.1.18), B-106: (I) + (B.1.19), B-107: (I) + (B.1.20), B-108: (I) + (B.1.21),
B-109: (I) + (B.1.22), B-110: (I) + (B.1.23), B-111: (I) + (B.1.24), B-112: (I) + (B.1.25), B-113: (I) + (B.1.26),
B-114: (I) + (B.1.27), B-115: (I) + (B.1.28), B-116: (I) + (B.1.29), B-117: (I) + (B.1.30), B-118: (I) + (B.1.31),
B-119: (I) + (B.1.32), B-120: (I) + (B.1.37), B-121: (I) + (B.1.38), B-122: (I) + (B.1.39), B-123: (I) + (B.1.40),
B-124: (I) + (B.1.41), B-125: (I) + (B.1.42), B-126: (I) + (B.1.43), B-127: (I) + (B.1.44), B-128:
(I) + (B.1.45), B-129: (I) + (B.1.46), B-130: (I) + (B.1.47), B-131: (I) + (B.1.48), B-132: (I) + (B.1.49),
B-133: (I) + (B.1.50), B-134: (I) + (B.1.51), B-135: (I) + (B.1.52), B-136: (I) + (B.2.1),
B-137: (I) + (B.2.2),
B-138: (I) + (B.2.3), B-139: (I) + (B.2.4), B-140: (I) + (B.2.5), B-141: (I) + (B.2.6), B-142: (I) + (B.2.7),
B-143: (I) + (B.2.8), B-144: (I) + (B.3.1), B-145: (I) + (B.4.1), B-146: (I) + (C.1.1), B-147: (I) + (C.1.2),
B-148: (I) + (C.1.3), B-149: (I) + (C.1.4), B-150: (I) + (C.1.5), B-151: (I) + (C.1.6), B-152: (I) + (C.1.7),
B-153: (I) + (C.2.1), B-154: (I) + (C.2.2), B-155: (I) + (C.2.3), B-156: (I) + (C.2.4), B-157: (I) + (C.2.5),
B-158: (I) + (C.2.6), B-159: (I) + (C.2.7), B-160: (I) + (C.2.8), B-161: (I) + (D.1.1), B-162: (I) + (D.1.2),
B-163: (I) + (D.1.3), B-164: (I) + (D.1.4), B-165: (I) + (D.1.5), B-166: (I) + (D.1.6), B-167: (I) + (D.1.7),
B-168: (I) + (D.1.8), B-169: (I) + (D.1.9), B-170: (I) + (D.1.10), B-171: (I) + (D.1.11), B-172:
(I) + (D.1.12), B-173: (I) + (D.1.13), B-174: (I) + (D.1.14), B-175: (I) + (D.1.15), B-176: (I) + (D.1.16),
B-177: (I) + (D.2.1), B-178: (I) + (D.2.2), B-179: (I) + (D.2.3), B-180: (I) + (D.2.4), B-181: (I) + (D.2.5),
B-182: (I) + (D.2.6), B-183: (I) + (D.2.7), B-184: (I) + (E.1.1), B-185: (I) + (E.1.2), B-186: (I) + (E.1.3),
B-187: (I) + (E.2.1), B-188: (I) + (E.2.2), B-189: (I) + (E.2.3), B-190: (I) + (E.2.4), B-191: (I) + (E.2.5),
B-192: (I) + (E.2.6), B-193: (I) + (F.1.1), B-194: (I) + (F.1.2), B-195: (I) + (F.1.3), B-196: (I) + (F.1.4),
B-197: (I) + (F.1.5), B-198: (I) + (F.2.1), B-199: (I) + (G.1.1), B-200: (I) + (G.1.2), B-201: (I) + (G.1.3),
B-202: (I) + (G.1.4), B-203: (I) + (G.2.1), B-204: (I) + (G.2.2), B-205: (I) + (G.2.3), B-206: (I) + (G.2.4),
B-207: (I) + (G.2.5), B-208: (I) + (G.2.6), B-209: (I) + (G.2.7), B-210: (I) + (G.3.1), B-211: (I) + (G.3.2),
B-212: (I) + (G.3.3), B-213: (I) + (G.3.4), B-214: (I) + (G.3.5), B-215: (I) + (G.3.6), B-216: (I) + (G.3.7),
B-217: (I) + (G.4.1), B-218: (I) + (G.5.1), B-219: (I) + (G.5.2), B-220: (I) + (G.5.3), B-221: (I) + (G.5.4),
B-222: (I) + (G.5.5), B-223: (I) + (G.5.6), B-224: (I) + (G.5.7), B-225: (I) + (G.5.8), B-226: (I) + (G.5.9),
B-227: (I) + (G.5.10), B-228: (I) + (G.5.11), B-229: (I) + (H.1.1), B-230: (I) + (H.1.2),
B-231: (I) + (H.1.3),
B-232: (I) + (H.1.4), B-233: (I) + (H.1.5), B-234: (I) + (H.1.6), B-235: (I) + (H.1.7), B-236: (I) + (H.2.1),
B-237: (I) + (H.2.2), B-238: (I) + (H.2.3), B-239: (I) + (H.2.4), B-240: (I) + (H.2.5), B-241: (I) + (H.2.6),
B-242: (I) + (H.2.7), B-243: (I) + (H.2.8), B-244: (I) + (H.2.9), B-245: (I) + (H.3.1), B-246: (I) + (H.3.2),
B-247: (I) + (H.3.3), B-248: (I) + (H.3.4), B-249: (I) + (H.3.5), B-250: (I) + (H.3.6), B-251: (I) + (H.3.7),
B-252: (I) + (H.3.8), B-253: (I) + (H.3.9), B-254: (I) + (H.3.10), B-255: (I) + (H.3.11), B-256:
(I) + (H.4.1), B-257: (I) + (H.4.2), B-258: (I) + (H.4.3), B-259: (I) + (H.4.4), B-260: (I) + (H.4.5), B-261:
(I) + (H.4.6), B-262: (I) + (H.4.7), B-263: (I) + (H.4.8), B-264: (I) + (H.4.9), B-265: (I) + (H.4.10),
B-266: (I) + (I.1.1), B-267: (I) + (I.1.2), B-268: (I) + (I.2.1),
B-269: (I) + (I.2.2), B-270: (I) + (I.2.3), B-271:
(I) + (I.2.4), B-272: (I) + (I.2.5), B-273: (I) + (J.1.1), B-274: (I) + (J.1.2), B-275: (I) + (J.1.3), B-276:
(I) + (J.1.4), B-277: (I) + (J.1.5), B-278: (I) + (J.1.6), B-279: (I) + (J.1.7), B-280: (I) + (J.1.8), B-281:
(I) + (J.1.9), B-282: (I) + (J.1.10), B-283: (I) + (J.1.11), B-284: (I) + (J.1.12), B-285: (I) + (K.1.1), B-286:
(I) + (K.1.2), B-287: (I) + (K.1.3), B-288: (I) + (K.1.4), B-289: (I) + (K.1.5), B-290: (I) + (K.1.6), B-291:
(I) + (K.1.7), B-292: (I) + (K.1.8), B-293: (I) + (K.1.9), B-294: (I) + (K.1.10), B-295: (I) + (K.1.11),
B-296: (I) + (K.1.12), B-297: (I) + (K.1.13),
B-298: (I) + (K.1.14), B-299: (I) + (K.1.15), B-300: (I) + (K.1.16),
B-301: (I) + (K.1.17), B-302: (I) + (K.1.18), B-303: (I) + (K.1.19), B-304: (I) + (K.1.20), B-305:
(I) + (K.1.21), B-306: (I) + (K.1.22), B-307: (I) + (K.1.23), B-308: (I) + (K.1.24), B-309: (I) + (K.1.25),
B-310: (I) + (K.1.26), B-311: (I) + (K.1.27), B-312: (I) + (K.1.28), B-313: (I) + (K.1.29), B-314:
(I) + (K.1.30), B-315: (I) + (K.1.31), B-316: (I) + (K.1.32), B-317: (I) + (K.1.33), B-318: (I) + (K.1.34),
B-319: (I) + (K.1.35), B-320: (I) + (K.1.36), B-321: (I) + (K.1.37), B-322: (I) + (K.1.38), B-323:
(I) + (K.1.39), B-324: (I) + (K.1.40), B-325: (I) + (K.1.41), B-326: (I) + (K.1.42), B-327: (I) + (K.1.43),
B-328: (I) + (K.1.44), B-329: (I) + (K.1.45), B-330: (I) + (K.1.46), B-331: (I) + (K.1.47), B-332:
(I) + (K.1.48), B-333: (I) + (K.1.49), B-334: (I) + (K.1.50), B-335: (I) + (K.1.51), B-336: (I) + (K.1.52),
B-337: (I) + (K.1.53), B-338: (I) + (K.1.54), B-339: (I) + (M.1.1), B-340: (I) + (N.1.1),
B-341: (I) + (N.1.2), B-342: (I) + (N.1.3), B-343: (I) + (N.1.4),
B-344: (I) + (N.1.5), B-345: (I) + (N.1.6), B-346: (I) + (N.1.7),
B-347: (I) + (N.1.8), B-348: (I) + (N.1.9), B-349: (I) + (N.1.10), B-350: (I) + (N.1.11), B-351:
(I) + (N.1.12), B-352: (I) + (N.1.13), B-353: (I) + (N.1.14), B-354: (I) + (N.1.15), B-355: (I) + (N.1.16),
B-356: (I) + (N.1.17), B-357: (I) + (N.1.18), B-358: (I) + (N.1.19), B-359: (I) + (N.1.20), B-360:
(I) + (N.1.21), B-361: (I) + (N.1.22), B-362: (I) + (N.1.23), B-363: (I) + (N.1.24), B-364: (I) + (N.1.25),
B-365: (I) + (N.1.26), B-366: (I) + (N.1.27), B-367: (I) + (N.1.28), B-368: (I) + (N.1.29), B-369:
(I) + (N.1.30), B-370: (I) + (N.1.31), B-371: (I) + (N.1.32), B-372: (I) + (N.1.33), B-373: (I) + (N.1.34),
B-374: (I) + (N.1.35), B-375: (I) + (N.1.36), B-376: (I) + (N.1.37), B-377: (I) + (N.1.38), B-378:
(I) + (N.1.39), B-379: (I) + (N.1.40), B-380: (I) + (N.1.41), B-381: (I) + (N.1.42), B-382: (I) + (N.1.43),
B-383: (I) + (N.1.44), B-384: (I) + (N.1.45), B-385: (I) + (N.1.46), B-386: (I) + (N.1.47), B-387:
(I) + (N.1.48), B-388: (I) + (N.1.49), B-389: (I) + (N.1.50), B-390: (I) + (N.1.51), B-391: (I) + (N.1.52),
B-392: (I) + (N.1.53), B-393: (I) + (N.1.54), B-394: (I) + (N.1.55), B-395: (I) + (N.1.56), B-396:
(I) + (N.1.57), B-397: (I) + (N.1.58), B-398: (I) + (N.1.59), B-399: (I) + (N.1.60), B-400: (I) + (N.1.61),
B-401: (I) + (N.1.62), B-402: (I) + (N.1.63), B-403: (I) + (N.1.64), B-404: (I) + (N.1.65), B-405:
(I) + (N.1.66), B-406: (I) + (N.2.1), B-407: (I) + (N.2.2), B-408: (I) + (N.2.3), B-409: (I) + (N.2.4),
B-410: (I) + (N.2.5), B-411: (I) + (N.2.6), B-412: (I) + (N.2.7),
B-413: (I) + (N.2.8), B-414: (I) + (N.2.9), B-415:
(I) + (N.2.10), B-416: (I) + (N.2.11), B-417: (I) + (N.2.12), B-418: (I) + (N.2.13), B-419: (I) + (N.2.14),
B-420: (I) + (N.2.15), B-421: (I) + (N.2.16), B-422: (I) + (N.2.17), B-423: (I) + (N.2.18), B-424:
(I) + (N.2.19), B-425: (I) + (N.2.20), B-426: (I) + (N.2.21), B-427: (I) + (N.2.22), B-428: (I) + (N.2.23),
B-429: (I) + (N.2.24), B-430: (I) + (N.2.25), B-431: (I) + (N.2.26), B-432: (I) + (N.2.27), B-433:
(I) + (N.2.28), B-434: (I) + (N.2.29), B-435: (I) + (N.2.30), B-436: (I) + (N.2.31), B-437: (I) + (N.2.32),
B-438: (I) + (N.2.33), B-439: (I) + (N.2.34), B-440: (I) + (N.2.35), B-441: (I) + (N.2.36), B-442:
(I) + (N.2.37), B-443: (I) + (N.2.38), B-444: (I) + (N.2.39), B-445: (I) + (N.2.40), B-446: (I) + (N.2.41),
B-447: (I) + (N.2.42), B-448: (I) + (N.2.43), B-449: (I) + (N.2.44), B-450: (I) + (N.2.45), B-451:
(I) + (N.2.46), B-452: (I) + (N.2.47), B-453: (I) + (N.2.48), B-454: (I) + (N.2.49), B-455: (I) + (N.2.50),
B-456: (I) + (N.2.51), B-457: (I) + (N.2.52), B-458: (I) + (N.2.53), B-459: (I) + (N.2.54), B-460:
(I) + (N.2.55), B-461: (I) + (N.2.56), B-462: (I) + (N.2.57), B-463: (I) + (N.2.58), B-464: (I) + (N.2.59),
B-465: (I) + (N.2.60), B-466: (I) + (N.2.61), B-467: (I) + (N.2.62), B-468: (I) + (N.2.63), B-469:
(I) + (N.2.64), B-470: (I) + (N.2.65), B-471: (I) + (N.2.66), B-472: (I) + (N.2.67), B-473: (I) + (N.2.68),
B-474: (I) + (N.2.69), B-475: (I) + (N.2.70), B-476: (I) + (N.2.71), B-477: (I) + (N.2.72), B-478:
(I) + (N.2.73), B-479: (I) + (N.2.74), B-480: (I) + (N.2.75), B-481: (I) + (N.2.76), B-482: (I) + (N.2.77),
B-483: (I) + (N.2.78), B-484: (I) + (N.2.79), B-485: (I) + (N.2.80), B-486: (I) + (N.2.81), B-487:
(I) + (N.2.82), B-488: (I) + (N.2.83), B-489: (I) + (N.2.84), B-490: (I) + (N.3.1), B-491: (I) + (N.3.2), B-
B-492: (I) + (N.3.3), B-493: (I) + (N.3.4), B-494: (I) + (N.3.5), B-495: (I) + (N.3.6), B-496: (I) + (N.3.7),
B-497: (I) + (N.3.8), B-498: (I) + (N.3.9), B-499: (I) + (N.3.10), B-500: (I) + (N.3.11),
B-501: (I) + (N.3.12),
B-502: (I) + (N.3.13), B-503: (I) + (N.3.14), B-504: (I) + (N.3.15), B-505: (I) + (N.3.16), B-506:
(I) + (N.3.17), B-507: (I) + (N.3.18), B-508: (I) + (N.3.19), B-509: (I) + (N.3.20), B-510: (I) + (N.3.21),
B-511: (I) + (N.3.22), B-512: (I) + (N.3.23), B-513: (I) + (N.3.24), B-514: (I) + (N.3.25), B-515:
(I) + (N.3.26), B-516: (I) + (N.3.27), B-517: (I) + (N.3.28), B-518: (I) + (N.3.29), B-519: (I) + (N.3.30),
B-520: (I) + (N.3.31), B-521: (I) + (N.3.32), B-522: (I) + (N.3.33), B-523: (I) + (N.3.34), B-524:
(I) + (N.3.35), B-525: (I) + (N.3.36), B-526: (I) + (N.3.37), B-527: (I) + (N.3.38), B-528: (I) + (N.3.39),
B-529: (I) + (N.3.40), B-530: (I) + (N.3.41), B-531: (I) + (N.3.42), B-532: (I) + (N.3.43), B-533:
(I) + (N.3.44), B-534: (I) + (N.3.45), B-535: (I) + (N.3.46), B-536: (I) + (N.3.47), B-537: (I) + (N.3.48),
B-538: (I) + (N.3.49), B-539: (I) + (N.3.50), B-540: (I) + (N.3.51), B-541: (I) + (N.3.52), B-542:
(I) + (N.3.53), B-543: (I) + (N.3.54), B-544: (I) + (N.3.55), B-545: (I) + (N.3.56), B-546: (I) + (N.3.57),
B-547: (I) + (N.3.58), B-548: (I) + (N.4.1), B-549: (I) + (N.4.2), B-550: (I) + (N.4.3), B-551: (I) + (N.4.4),
B-552: (I) + (N.4.5), B-553: (I) + (N.4.6), B-554: (I) + (N.4.7), B-555: (I) + (N.4.8), B-556: (I) + (N.4.9),
B-557: (I) + (N.4.10), B-558: (I) + (N.4.11), B-559: (I) + (N.4.12), B-560: (I) + (N.4.13), B-561:
(I) + (N.4.14), B-562: (I) + (N.4.15), B-563: (I) + (N.4.16), B-564: (I) + (N.4.17), B-565: (I) + (N.4.18),
B-566: (I) + (N.4.19), B-567: (I) + (N.4.20), B-568: (I) + (N.4.21), B-569: (I) + (N.4.22), B-570:
(I) + (N.4.23), B-571: (I) + (N.4.24), B-572: (I) + (N.4.25), B-573: (I) + (N.4.26), B-574: (I) + (N.4.27),
B-575: (I) + (N.4.28), B-576: (I) + (N.4.29), B-577: (I) + (N.4.30), B-578: (I) + (N.4.31), B-579:
(I) + (N.4.32), B-580: (I) + (N.4.33), B-581: (I) + (N.4.34), B-582: (I) + (N.4.35), B-583: (I) + (N.4.36),
B-584: (I) + (N.4.37), B-585: (I) + (N.4.38), B-586: (I) + (N.4.39), B-587: (I) + (N.4.40), B-588:
(I) + (N.4.41), B-589: (I) + (N.4.42), B-590: (I) + (N.4.43), B-591: (I) + (N.4.44), B-592: (I) + (N.4.45),
B-593: (I) + (N.4.46), B-594: (I) + (N.4.47), B-595: (I) + (N.5.1), B-596: (I) + (N.5.2), B-597: (I) + (N.5.3),
B-598: (I) + (N.5.4), B-599: (I) + (N.5.5), B-600: (I) + (N.5.6), B-601: (I) + (N.5.7), B-602: (I) + (N.5.8),
B-603: (I) + (N.5.9), B-604: (I) + (N.5.10), B-605: (I) + (N.5.11), B-606: (I) + (N.5.12), B-607:
(I) + (N.5.13), B-608: (I) + (N.5.14), B-609: (I) + (N.5.15), B-610: (I) + (N.5.16), B-611: (I) + (N.5.17),
B-612: (I) + (N.5.18), B-613: (I) + (N.5.19), B-614: (I) + (N.5.20), B-615: (I) + (N.5.21), B-616:
(I) + (N.5.22), B-617: (I) + (N.5.23), B-618: (I) + (N.5.24), B-619: (I) + (N.5.25), B-620: (I) + (N.5.26),
B-621: (I) + (N.6.1), B-622: (I) + (N.6.2), B-623: (I) + (N.6.3), B-624: (I) + (N.6.4), B-625: (I) + (N.7.1),
B-626: (I) + (N.7.2), B-627: (I) + (N.7.3), B-628: (I) + (N.7.4), B-629: (I) + (N.7.5), B-630: (I) + (N.8.1),
B-631: (I) + (N.9.1), B-632: (I) + (N.9.2), B-633: (I) + (N.9.3), B-634: (I) + (N.9.4), B-635: (I) + (N.9.5),
B-636: (I) + (N.9.6), B-637: (I) + (N.9.7), B-638: (I) + (N.9.8), B-639: (I) + (N.9.9),
B-640: (I) + (N.9.10),
B-641: (I) + (N.9.11), B-642: (I) + (N.9.12), B-643: (I) + (N.9.13), B-644: (I) + (N.9.14), B-645:
(I) + (N.9.15), B-646: (I) + (N.9.16), B-647: (I) + (N.9.17), B-648: (I) + (N.9.18), B-649: (I) + (N.9.19),
B-650: (I) + (N.9.20), B-651: (I) + (N.9.21), B-652: (I) + (N.9.22), B-653: (I) + (N.9.23), B-654:
(I) + (N.9.24), B-655: (I) + (N.9.25), B-656: (I) + (N.9.26), B-657: (I) + (N.10.1), B-658: (I) + (N.10.2),
B-659: (I) + (N.10.3), B-660: (I) + (N.10.4), B-661: (I) + (N.10.5), B-662: (I) + (N.10.6), B-663:
(I) + (N.10.7), B-664: (I) + (N.10.8), B-665: (I) + (N.10.9), B-666: (I) + (N.10.10), B-667: (I) + (N.10.11),
B-668: (I) + (N.10.12), B-669: (I) + (N.10.13), B-670: (I) + (N.10.14), B-671: (I) + (N.10.15), B-672:
(I) + (N.10.16), B-673: (I) + (N.10.17), B-674: (I) + (N.10.18), B-675: (I) + (N.10.19), B-676:
(I) + (N.10.20), B-677: (I) + (N.10.21), B-678: (I) + (N.10.22), B-679: (I) + (N.10.23), B-680:
(I) + (N.10.24), B-681: (I) + (N.10.25), B-682: (I) + (N.10.26), B-683: (I) + (N.10.27), B-684:
(I) + (N.10.28), B-685: (I) + (II.1), B-686: (I) + (II.2), B-687: (I) + (II.3), B-688: (I) + (II.4), B-689:
(I) + (II.5), B-690: (I) + (II.6), B-691: (I) + (II.7), B-692: (I) + (II.8), B-693: (I) + (II.9),
B-694: (I) + (N.11.1),
B-695: (I) + (N.11.2), B-696: (I) + (N.11.3), B-697: (I) + (N.11.4), B-698: (I) + (N.11.5), B-699:
(I) + (N.11.6), B-700: (I) + (N.11.7), B-701: (I) + (N.12.1), B-702: (I) + (N.12.2), B-703: (I) + (N.12.3),
B-704: (I) + (N.13.1), B-705: (I) + (N.13.2), B-706: (I) + (N.13.3), B-707: (I) + (N.13.4), B-708:
(I) + (N.13.5), B-709: (I) + (N.13.6), B-710: (I) + (N.13.7), B-711: (I) + (N.13.8), B-712: (I) + (N.13.9),
B-713: (I) + (N.13.10), B-714: (I) + (N.13.11), B-715: (I) + (N.13.12), B-716: (I) + (N.13.13), B-717:
(I) + (N.13.14), B-718: (I) + (N.13.15), B-719: (I) + (N.13.16), B-720: (I) + (N.13.17), B-721:
(I) + (N.13.18), B-722: (I) + (N.13.19), B-723: (I) + (N.13.20), B-724: (I) + (N.13.21), B-725:
(I) + (N.13.22), B-726: (I) + (N.13.23), B-727: (I) + (N.13.24), B-728: (I) + (N.13.25), B-729:
(I) + (N.13.26), B-730: (I) + (N.13.27), B-731: (I) + (N.13.28), B-732: (I) + (N.13.29), B-733:
(I) + (N.14.1), B-734: (I) + (N.14.2), B-735: (I) + (N.14.3), B-736: (I) + (N.14.4), B-737: (I) + (N.15.1),
B-738: (I) + (N.15.2), B-739: (I) + (N.15.3), B-740: (I) + (N.15.4), B-741: (I) + (N.15.5), B-742:
(I) + (N.15.6), B-743: (I) + (N.15.7), B-744: (I) + (N.15.8), B-745: (I) + (N.15.9), B-746: (I) + (N.15.10),
B-747: (I) + (N.15.11), B-748: (I) + (N.15.12), B-749: (I) + (N.15.13), B-750: (I) + (N.15.14), B-751:
(I) + (N.15.15), B-752: (I) + (N.15.16), B-753: (I) + (N.15.17), B-754: (I) + (N.15.18), B-755:
(I) + (N.15.19), B-756: (I) + (N.15.20), B-757: (I) + (N.15.21), B-758: (I) + (N.15.22), B-759:
(I) + (N.15.23), B-760: (I) + (N.15.24), B-761: (I) + (N.15.25), B-762: (I) + (N.15.26), B-763:
(I) + (N.15.27), B-764: (I) + (N.15.28), B-765: (I) + (N.15.29), B-766: (I) + (N.15.30), B-767:
(I) + (N.15.31), B-768: (I) + (N.15.32), B-769: (I) + (N.15.33), B-770: (I) + (N.15.34), B-771:
(I) + (N.15.35), B-772: (I) + (O.1.1), B-773: (I) + (O.1.2), B-774: (I) + (O.1.3), B-775: (I) + (O.1.4),
B-776: (I) + (O.1.5), B-777: (I) + (O.1.6), B-778: (I) + (O.1.7), B-779: (I) + (O.1.8), B-780: (I) + (O.1.9),
B-781: (I) + (O.1.10), B-782: (I) + (O.1.11), B-783: (I) + (O.1.12), B-784: (I) + (O.1.13), B-785:
(I) + (O.1.14), B-786: (I) + (O.1.15), B-787: (I) + (O.1.16), B-788: (I) + (O.1.17), B-789: (I) + (O.1.18),
B-790: (I) + (O.1.19), B-791: (I) + (O.1.20), B-792: (I) + (O.1.21), B-793: (I) + (O.1.22), B-794:
(I) + (O.1.23), B-795: (I) + (O.1.24), B-796: (I) + (O.1.25), B-797: (I) + (O.1.26), B-798: (I) + (O.1.27),
B-799: (I) + (O.1.28), B-800: (I) + (O.1.29), B-801: (I) + (O.1.30), B-802: (I) + (O.1.31), B-803:
(I) + (O.1.32), B-804: (I) + (O.1.33), B-805: (I) + (O.1.34), B-806: (I) + (O.1.35), B-807: (I) + (O.1.36),
B-808: (I) + (O.1.37), B-809: (I) + (O.1.38), B-810: (I) + (O.1.39), B-811: (I) + (O.1.40), B-812:
(I) + (O.1.41), B-813: (I) + (O.1.42), B-814: (I) + (O.1.43), B-815: (I) + (O.1.44), B-816: (I) + (O.1.45),
B-817: (I) + (O.1.46), B-818: (I) + (O.1.47), B-819: (I) + (O.1.48), B-820: (I) + (O.1.49), B-821:
(I) + (O.1.50), B-822: (I) + (O.1.51), B-823: (I) + (O.1.52), B-824: (I) + (O.1.53), B-825: (I) + (O.1.54),
B-826: (I) + (O.1.55), B-827: (I) + (O.1.56), B-828: (I) + (O.1.57), B-829: (I) + (O.1.58), B-830:
(I) + (O.1.59), B-831: (I) + (O.1.60), B-832: (I) + (O.1.61), B-833: (I) + (O.1.62), B-834: (I) + (O.1.63),
B-835: (I) + (O.1.64), B-836: (I) + (O.1.65), B-837: (I) + (O.1.66), B-838: (I) + (O.1.67), B-839:
(I) + (O.1.68), B-840: (I) + (O.1.69), B-841: (I) + (O.1.70), B-842: (I) + (O.1.71), B-843: (I) + (O.1.72),
B-844: (I) + (O.1.73), B-845: (I) + (O.1.74), B-846: (I) + (O.1.75), B-847: (I) + (O.1.76), B-848:
(I) + (O.1.77), B-849: (I) + (O.1.78), B-850: (I) + (O.1.79), B-851: (I) + (O.1.80), B-852: (I) + (O.1.81),
B-853: (I) + (O.1.82), B-854: (I) + (O.1.83), B-855: (I) + (O.1.84), B-856: (I) + (O.1.85), B-857:
(I) + (O.1.86), B-858: (I) + (O.1.87), B-859: (I) + (O.1.88), B-860: (I) + (O.1.89), B-861: (I) + (O.1.90),
B-862: (I) + (O.1.91), B-863: (I) + (O.2.1), B-864: (I) + (O.2.2), B-865: (I) + (O.2.3), B-866: (I) + (O.2.4),
B-867: (I) + (O.2.5), B-868: (I) + (O.2.6), B-869: (I) + (O.2.7), B-870: (I) + (O.3.1), B-871: (I) + (O.3.2),
B-872: (I) + (O.3.3), B-873: (I) + (O.3.4), B-874: (I) + (O.3.5), B-875: (I) + (O.3.6), B-876: (I) + (O.3.7),
B-877: (I) + (O.3.8), B-878: (I) + (O.3.9), B-879: (I) + (O.3.10), B-880: (I) + (O.3.11), B-881:
(I) + (O.3.12), B-882: (I) + (O.3.13), B-883: (I) + (O.3.14), B-884: (I) + (O.3.15), B-885: (I) + (O.3.16),
B-886: (I) + (O.3.17), B-887: (I) + (O.3.18), B-888: (I) + (O.3.19), B-889: (I) + (O.3.20), B-890:
(I) + (O.3.21), B-891: (I) + (O.3.22), B-892: (I) + (O.3.23), B-893: (I) + (O.3.24), B-894: (I) + (O.3.25),
B-895: (I) + (O.3.26), B-896: (I) + (O.3.27), B-897: (I) + (O.3.28), B-898: (I) + (O.3.29), B-899:
(I) + (O.3.30), B-900: (I) + (O.3.31), B-901: (I) + (O.3.32), B-902: (I) + (O.3.33), B-903: (I) + (O.3.34),
B-904: (I) + (O.3.35), B-905: (I) + (O.3.36), B-906: (I) + (O.3.37), B-907: (I) + (O.3.38), B-908:
(I) + (O.3.39), B-909: (I) + (O.3.40), B-910: (I) + (O.3.41), B-911: (I) + (O.3.42), B-912: (I) + (O.3.43),
B-913: (I) + (O.3.44), B-914: (I) + (O.3.45), B-915: (I) + (O.3.46), B-916: (I) + (O.3.47), B-917:
(I) + (O.3.48), B-918: (I) + (O.3.49), B-919: (I) + (O.4.1), B-920: (I) + (O.4.2), B-921: (I) + (O.4.3),
B-922: (I) + (O.4.4), B-923: (I) + (O.4.5), B-924: (I) + (O.4.6), B-925: (I) + (O.4.7), B-926: (I) + (O.4.8),
B-927: (I) + (O.4.9), B-928: (I) + (O.4.10), B-929: (I) + (O.4.11), B-930: (I) + (O.4.12), B-931:
(I) + (O.4.13), B-932: (I) + (O.4.14), B-933: (I) + (O.5.1), B-934: (I) + (O.5.2), B-935: (I) + (O.6.1),
B-936: (I) + (O.6.2), B-937: (I) + (O.6.3), B-938: (I) + (O.6.4), B-939: (I) + (O.6.5), B-940: (I) + (O.7.1),
B-941: (I) + (O.7.2), B-942: (I) + (O.7.3), B-943: (I) + (O.7.4), B-944: (I) + (O.7.5), B-945: (I) + (O.8.1),
B-946: (I) + (O.8.2), B-947: (I) + (O.8.3), B-948: (I) + (O.8.4), B-949: (I) + (O.8.5), B-950: (I) + (O.9.1),
B-951: (I) + (O.9.2), B-952: (I) + (O.9.3), B-953: (I) + (O.10.1), B-954: (I) + (O.10.2),
B-955: (I) + (O.10.3),
B-956: (I) + (O.10.4), B-957: (I) + (O.12.1), B-958: (I) + (O.12.2), B-959: (I) + (O.12.3), B-960:
(I) + (O.12.4), B-961: (I) + (O.12.5), B-962: (I) + (O.12.6), B-963: (I) + (O.13.1), B-964: (I) + (O.13.2),
B-965: (I) + (O.13.3), B-966: (I) + (O.14.1), B-967: (I) + (O.14.2), B-968: (I) + (O.14.3), B-969:
(I) + (O.14.4), B-970: (I) + (O.15.1), B-971: (I) + (O.15.2), B-972: (I) + (O.15.3), B-973: (I) + (O.15.4),
B-974: (I) + (O.15.5), B-975: (I) + (O.15.6), B-976: (I) + (O.15.7), B-977: (I) + (O.15.8), B-978:
(I) + (O.15.9), B-979: (I) + (O.15.10), B-980: (I) + (O.15.11), B-981: (I) + (O.16.1), B-982: (I) + (O.17.1),
B-983: (I) + (O.18.1), B-984: (I) + (O.18.2), B-985: (I) + (O.18.3), B-986: (I) + (O.18.4), B-987:
(I) + (O.18.5), B-988: (I) + (O.19.1), B-989: (I) + (O.20.1), B-990: (I) + (O.21.1), B-991: (I) + (O.21.2),
B-992: (I) + (O.21.3), B-993: (I) + (O.21.4), B-994: (I) + (O.21.5), B-995: (I) + (O.21.6), B-996:
(I) + (O.21.7), B-997: (I) + (O.22.1), B-998: (I) + (O.22.2), B-999: (I) + (O.22.3), B-1000: (I) + (O.22.4),
B-1001: (I) + (O.23.1), B-1002: (I) + (O.23.2), B-1003: (I) + (O.23.3), B-1004: (I) + (O.23.4), B-1005:
(I) + (O.24.1), B-1006: (I) + (O.24.2), B-1007: (I) + (O.24.3), B-1008: (I) + (O.24.4), B-1009:
(I) + (O.24.5), B-1010: (I) + (O.25.1), B-1011: (I) + (O.25.2), B-1012: (I) + (O.26.1), B-1013:
(I) + (O.26.2), B-1014: (I) + (O.26.3), B-1015: (I) + (O.26.4), B-1016: (I) + (O.26.5), B-1017:
(I) + (O.26.6), B-1018: (I) + (O.26.7), B-1019: (I) + (O.26.8), B-1020: (I) + (O.26.9), B-1021:
(I) + (O.26.10), B-1022: (I) + (O.26.11), B-1023: (I) + (O.26.12), B-1024: (I) + (O.26.13), B-1025:
(I) + (O.26.14), B-1026: (I) + (O.26.15), B-1027: (I) + (O.26.16), B-1028: (I) + (O.26.17), B-1029:
(I) + (O.26.18), B-1030: (I) + (O.27.1), B-1031: (I) + (O.27.2), B-1032: (I) + (O.27.3), B-1033:
(I) + (O.27.4), B-1034: (I) + (O.27.5), B-1035: (I) + (O.27.7), B-1036: (I) + (O.27.8), B-1037:
(I) + (O.27.9), B-1038: (I) + (O.27.10), B-1039: (I) + (O.27.11), B-1040: (I) + (O.27.12), B-1041:
(I) + (O.27.13), B-1042: (I) + (O.27.14), B-1043: (I) + (O.27.15), B-1044: (I) + (O.27.16), B-1045:
(I) + (O.27.17), B-1046: (I) + (O.27.18), B-1047: (I) + (O.27.19), B-1048: (I) + (O.27.20), B-1049:
(I) + (O.27.21), B-1050: (I) + (O.27.22), (I) + (O.27.23), (I) + (O.27.24), B-1051: B-1052: B-1053:
(I) + (O.27.25), B-1054: (I) + (O.27.26), B-1055: (I) + (O.27.27), B-1056: (I) + (O.27.28), B-1057:
(I) + (O.27.29), B-1058: (I) + (O.27.30), B-1059: (I) + (O.27.31), B-1060: (I) + (O.27.42), B-1061:
(I) + (O.27.43), B-1062: (I) + (O.27.44), B-1063: (I) + (O.27.45), B-1064: (I) + (O.27.46), B-1065:
(I) + (O.27.47), B-1066: (I) + (O.27.48), B-1067: (I) + (O.27.49), B-1068: (I) + (O.27.50), B-1069:
(I) + (O.27.51), B-1070: (I) + (O.27.52), B-1071: (I) + (O.27.53), B-1072: (I) + (O.27.54), B-1073:
(I) + (O.27.55), B-1074: (I) + (O.27.56), B-1075: (I) + (O.27.57), B-1076: (I) + (O.27.58), B-1077:
(I) + (O.27.59), B-1078: (I) + (O.27.60), (I) + (O.27.61), (I) + (O.27.62), B-1079: B-1080: B-1081:
(I) + (O.27.63), B-1082: (I) + (O.27.64), B-1083: (I) + (O.27.65), B-1084: (I) + (O.27.66), B-1085:
(I) + (O.27.67), B-1086: (I) + (O.27.68), B-1087: (I) + (O.27.69), B-1088: (I) + (O.27.70), B-1089:
(I) + (O.27.71), B-1090: (I) + (O.27.72), B-1091: (I) + (O.27.73), B-1092: (I) + (O.27.74), B-1093:
(I) + (O.27.75), B-1094: (I) + (O.27.76), B-1095: (I) + (O.27.77), B-1096: (I) + (O.27.78), B-1097:
(I) + (O.27.79), B-1098: (I) + (O.27.80), B-1099: (I) + (O.27.81), B-1100: (I) + (O.27.82), B-1101:
(I) + (O.27.83), B-1102: (I) + (O.27.84), B-1103: (I) + (O.27.85), B-1104: (I) + (O.27.86), B-1105:
(I) + (O.27.87), B-1106: (I) + (O.27.88), B-1107: (I) + (O.27.89), B-1108: (I) + (O.27.90), B-1109:
(I) + (O.27.91), B-1110: (I) + (O.27.92), B-1111: (I) + (O.27.93), B-1112: (I) + (O.27.94), B-1113:
(I) + (O.27.95), B-1114: (I) + (O.27.96), B-1115: (I) + (O.27.97), B-1116: (I) + (O.27.98), B-1117:
(I) + (O.27.99), B-1118: (I) + (O.27.100), B-1119: (I) + (O.27.101), B-1120: (I) + (O.27.102), B-1121:
(I) + (O.27.103), B-1122: (I) + (O.27.104), B-1123: (I) + (O.27.105), B-1124: (I) + (O.27.106), B-1125:
(I) + (O.27.107), B-1126: (I) + (O.27.108), BB-1127: (I) + (O.27.109), B-1128: I(I) + (O.27.110).

The mixtures of active substances can be prepared as compositions comprising besides the active ingredients at least one inert ingredient (auxiliary) by usual means, e. g. by the means given for the compositions of compounds I.

Concerning usual ingredients of such compositions reference is made to the explanations given for the compositions containing compounds I.

The mixtures of active substances according to the present invention are suitable as fungicides, as are the compounds of formula I. They are distinguished by an outstanding effectiveness against a broad spectrum of phytopathogenic fungi, especially from the classes of the Ascomycetes, Basidiomycetes, Deuteromycetes and Peronosporomycetes (syn. Oomycetes). In addition, it is refered to the explanations regarding the fungicidal activity of the compounds and the compositions containing compounds I, respectively.

### I. SYNTHESIS EXAMPLES

The compounds of formula I can be prepared according to the methods outlined below.

### I.1) Preparation of 4-(methylaminomethyl)benzonitrile

To a solution of 4-(bromomethyl)benzonitrile (49 g, 1 eq.) in tetrahydrofuran (500 mL) methylamine (194 g of a 40% solution by weight, 10 eq.) was added. The mixture was stirred overnight at room temperature. After removing the solvent under reduced pressure, an aqueous solution of sodium chloride was added and the aqueous mixture was extracted with ethyl acetate. The combined organic layer was dried over magnesium sulfate and freed from solvent.

The title compound (35.6 g) was used directly without further purification.

### I.2) Preparation of N-[(4-cyanophenyl)methyl]-2,2,2-trifluoro-N-methyl-ethanesulfonamide

To a solution of 4-(methylaminomethyl)benzonitrile (5.0 g, 1 eq.) and triethylamine (8.65 g, 2.5 eq.) in dichloromethane (100 mL) was added 2,2,2-trifluoroethanesulfonyl chloride (7.49 g, 1.2 eq.). The mixture was stirred at room temperature for two hours. Then ethyl acetate was added, the organic layer was washed with water, dried over sodium sulfate and concentrated in vacuo to yield the title compound (10.4 g) as yellow solid witch was used without further purification.

### I.3) Preparation of N-hydroxy-4-[[methyl(2,2,2-trifluoroethylsulfonyl)amino]methyl]benzamidine

To a solution of N-[(4-cyanophenyl)methyl]-2,2,2-trifluoro-N-methyl-ethanesulfonamide (10.4 g, 1 eq.) in ethanol (200 mL) was added hydroxylammonium hydrochloride (4.95 g, 2 eq.), then triethylamine (9.0 g, 2.5 eq.). The mixture was stirred at room temperature for 4 hours. After removing the solvent under reduced pressure, the crude product was used without further purification.

### I.4) Preparation of 2,2,2-trifluoro-N-methyl-N-[[4-[5-(trifluoromethyl)-1,2,4-oxadiazol-3-yl]phenyl]methyl]ethanesulfonamide (Ex-2)

To a solution of N-hydroxy-4-[[methyl(2,2,2-trifluoroethylsulfonyl)amino]methyl]benzamidine (0.86 g, 1 eq.) in dichloromethane (10 mL) trifluoroacetic anhydride (0.98 g, 4 eq.) was added. After stirring over night at room temperature the mixture was extracted with an aqueous solution of sodium bicarbonate, dried over sodium sulfate and concentrated in vacuo. The crude product was purified by flash chromatography to yield the title compound as yellow solid (0.15 g, 30%) (Ex-2). ¹H NMR (CDCl₃, 500 MHz): δ [ppm]=8.18 (d, 2H), 7.55 (d, 2H), 4,48 (s, 2H), 3.86 (q, 2H), 2.88 (s, 3H).

The compounds listed in Table I were prepared in analogy to the procedures described above.

TABLE I
Compounds Ex-1 to Ex-15 of formula I.F, wherein the
meaning of radicals R¹ and R² is as defined in each line.

| Ex. | | | HPLC Rt | Melting |
|---|---|---|---|---|
| no | R² | R¹ | (min)* | point (° C.) |
| Ex-1 | —CH₃ | —CH₂CF₂CH₃ | 1.228 | 92 |
| Ex-2 | —CH₃ | —CH₂CF₃ | 1.268 | 115 |
| Ex-3 | —CH₃ | —CH(CF₃)—CH₃ | 1.291 | 110 |
| Ex-4 | —CH₃ | —CH₂CHF₂ | 1.228 | 92 |
| Ex-5 | —CH₂CH₃ | —CH₂CF₃ | 1.300 | 97 |
| Ex-6 | —CH₂CH₃ | —CH(CF₃)—CH₃ | 1.321 | 110 |
| Ex-7 | —CH₂CH₃ | —CH₂CHF₂ | 1.263 | 76 |
| Ex-8 | —CH₂CH₃ | —CH₂CF₂CH₃ | 1.269 | 88 |
| Ex-9 | cyclopropyl | —CH₂CF₃ | 1.321 | 108 |
| Ex-10 | cyclopropyl | —CH(CF₃)—CH₃ | 1.336 | 83 |
| Ex-11 | cyclopropyl | —CH₂CHF₂ | 1.254 | 62 |
| Ex-12 | cyclopropyl | —CH₂CF₂CH₃ | 1.284 | 90 |
| Ex-13 | allyl | —CH₂CF₃ | 1.272 | 124 |
| Ex-14 | allyl | —CH₂CHF₂ | 1.263 | 96 |
| Ex-15 | allyl | —CH₂CF₃ | 1.331 | 73 |

*HPLC: High Performance Liquid Chromatography; HPLC-column Kinetex XB C18 1.7 μ (50 × 2.1 mm); eluent: acetonitrile/water + 0.1% trifluoroacetic acid (gradient from 5:95 to 100: 0 in 1.5 min at 60° C., flow gradient from 0.8 to 1.0 ml/min in 1.5 min). MS: Quadrupol Electrospray Ionisation, 80 V (positive mode). Rₜ: retention time in minutes.

### II. BIOLOGICAL EXAMPLES FOR FUNGICIDAL ACTIVITY

The fungicidal action of the compounds of formula I was demonstrated by the following experiments:

A. Glass House Trials

The spray solutions were prepared in several steps: The stock solution were prepared: a mixture of acetone and/or dimethylsulfoxide and the wetting agent/emulsifier Wettol, which is based on ethoxylated alkylphenoles, in a relation (by volume) solvent to emulsifier of 99 to 1, was added to 25 mg of the compound to give a total of 5 ml. Water was then added to total volume of 100 ml. This stock solution was diluted with the described solvent-emulsifier-water mixture to the given concentration.

A.1. Curative Control of Soy Bean Rust on Soy Beans Caused by Phakopsora pachyrhizi

Leaves of pot-grown soy bean seedlings were inoculated with spores of Phakopsora pachyrhizi. To ensure the success of the artificial inoculation, the plants were transferred to a humid chamber with a relative humidity of about 95% and 20 to 24° C. for 24 hours. The next day the plants were cultivated for 3 days in a greenhouse chamber at 23 to 27° C. and a relative humidity between 60 and 80%. Then the plants were sprayed to run-off with an aqueous suspension, containing the concentration of active ingredient or their mixture as described below. The plants were allowed to air-dry. Then the trial plants were cultivated for 14 days in a greenhouse chamber at 23 to 27° C. and a relative humidity between 60 and 80%. The extent of fungal attack on the leaves was visually assessed as % diseased leaf area.

In this test, the plants which had been treated with 16 ppm of the active compounds Ex-1 and Ex-2 showed a diseased leaf area of at most 5%, whereas the untreated plants showed 90% diseased leaf area.

A.2. Protective Control of Soy Bean Rust on Soy Beans Caused by Phakopsora pachyrhizi

Leaves of pot-grown soy bean seedlings were sprayed to run-off with an aqueous suspension, containing the concentration of active ingredient or their mixture as described below. The plants were allowed to air-dry. The trial plants were cultivated for 2 days in a greenhouse chamber at 23 to 27° C. and a relative humidity between 60 and 80%. Then the plants were inoculated with spores of Phakopsora pachyrhizi. To ensure the success the artificial inoculation, the plants were transferred to a humid chamber with a relative humidity of about 95% and 20 to 24° C. for 24 hours. The trial plants were cultivated for fourteen days in a greenhouse chamber at 23 to 27° C. and a relative humidity between 60 and 80%. The extent of fungal attack on the leaves was visually assessed as % diseased leaf area.

In this test, the plants which had been treated with 16 ppm of the active compounds Ex-1 and Ex-2 showed a diseased leaf area of at most 3%, whereas the untreated plants showed 90% diseased leaf area.

4. Preventative Control of Brown Rust on Wheat Caused by Puccinia recondita

The first two developed leaves of pot-grown wheat seedling were sprayed to run-off with an aqueous suspension, containing the concentration of active ingredient or their mixture as described below. The next day the plants were inoculated with spores of Puccinia recondita. To ensure the success the artificial inoculation, the plants were transferred to a humid chamber without light and a relative humidity of 95 to 99% and 20 to 24° C. for 24 hours. Then the trial plants were cultivated for 6 days in a greenhouse chamber at 20 to 24° C. and a relative humidity between 65 and 70%. The extent of fungal attack on the leaves was visually assessed as % diseased leaf area.

In this test, the plants which had been treated with 63 ppm of the active compounds Ex-1 and Ex-2 showed a diseased leaf area of at most 4%, whereas the untreated plants showed 80% diseased leaf area.

1.-13. (canceled)

14. A compound of formula I, or its N-oxide or an agriculturally acceptable salt thereof, 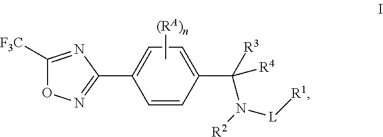
wherein:
R^{A} is independently selected from the group consisting of halogen, cyano, C₁-C₆-alkyl, C₁-C₆-haloalkyl, C₁-C₆-alkoxy or C₁-C₆-haloalkoxy;
n is 0, 1 or 2;
L is —S(═O)ₚ—;
p is 0, 1 or 2;
R¹ is C₁-C₆-alkyl; and wherein the alkyl group is substituted with 1, 2, 3, 4, 5 or up to the maximum possible number of identical or different halogen atoms; and wherein the alkyl group is further unsubstituted or, in addition to the halogen atoms, substituted with 1, 2, 3 or up to the maximum possible number of identical or different radicals selected from the group consisting of cyano, C₁-C₆-alkyl, C₁-C₆-alkoxy and C₃-C₈-cycloalkyl; and wherein the cycloalkyl group is unsubstituted or substituted with 1, 2, 3 or 4 or up to the maximum possible number of halogen atoms;
R² is hydrogen, C₁-C₆-alkyl, C₂-C₆-alkenyl, C₂-C₆-alkynyl, C₁-C₆-alkoxy, C₃-C₈-cycloalkyl, C₃-C₈-cycloalkenyl, C₃-C₈-cycloalkyl-C₁-C₆-alkyl, phenyl-C₁-C₄-alkyl, phenyl, C(═O)—(C₁-C₆-alkyl) or C(═O)—(C₁-C₆-alkoxy); and wherein any of the aliphatic or cyclic groups are unsubstituted or substituted with 1, 2, 3 or up to the maximum possible number of identical or different radicals selected from the group consisting of halogen, cyano, C₁-C₆-alkyl, C₁-C₆-alkoxy and C₃-C₈-cycloalkyl;
R³, R⁴ independently of each other are selected from the group consisting of hydrogen, halogen, cyano, C₁-C₄-alkyl, C₁-C₄-alkenyl, C₁-C₄-alkynyl, C₁-C₄-haloalkyl and C₁-C₄-alkoxy;
or R³ and R⁴ together with the carbon atom to which they are bound form a saturated 3- to 7-membered carbocycle or a saturated 3- to 6-membered heterocycle; wherein the saturated heterocycle includes beside carbon atoms 1, 2 or 3 heteroatoms independently selected from the group consisting of N, O and S as ring member atoms; and wherein said N ring member atom is substituted with the group R^{N}; and wherein
R^{N} is hydrogen, C₁-C₆-alkyl or halogen;
and wherein said S ring member atom is unsubstituted or substituted with 1 or 2 oxo radicals; and wherein one or two CH₂ groups of the saturated carbocycle or of the saturated heterocycle may be replaced by one or two groups independently selected from —C(═O)— and —C(═S)—; and wherein the carbon ring member atoms of the saturated carbocycle or the saturated heterocycle are unsubstituted or substituted with a total number of 1, 2, 3, 4 or up to the maximum possible number of identical or different radicals selected from the group consisting of halogen, cyano, C₁-C₆-alkyl, C₁-C₆-alkoxy and C₃-C₈-cycloalkyl.

15. The compound of claim 14, wherein n is 0 or 1; and wherein R^{A} is fluorine, chlorine, methyl or ethyl.

16. The compound of claim 14, wherein R³ and R⁴ are independently selected from hydrogen, halogen, C₁-C₆-alkyl or C₁-C₆-haloalkyl.

17. The compound of claim 14, wherein L is —S(═O)₂—.

18. The compound of claim 14, wherein R² is hydrogen, C₁-C₆-alkyl, C₂-C₆-alkenyl or C₃-C₈-cycloalkyl.

19. The compound of claim 14, wherein R² is hydrogen, C₁-C₆-alkyl, C₁-C₆-alkenyl, C₃-C₈-cycloalkyl, C₃-C₈-cycloalkyl-C₁-C₄-alkyl; preferably hydrogen, methyl, ethyl, iso-propyl, cyclopropyl, CH₂-cyclopropyl or allyl; and R¹ is C₃-C₈-cycloalkyl-C₁-C₂-alkyl; and wherein any of the alkyl or cycloalkyl group in R¹ are unsubstituted or substituted with 1, 2, 3 or 4 or up to the maximum possible number of radicals selected from the group consisting of chlorine and fluorine.

20. The compound of claim 14, wherein R² is hydrogen, C₁-C₆-alkyl, C₂-C₆-alkenyl or C₃-C₈-cycloalkyl; and R¹ is C₁-C₆-alkyl; and wherein the alkyl group in R¹ is substituted with 1, 2, 3 or up to the maximum possible number of atoms selected from the group consisting of fluorine and chlorine.

21. Intermediate compounds of the formula VI or VII, 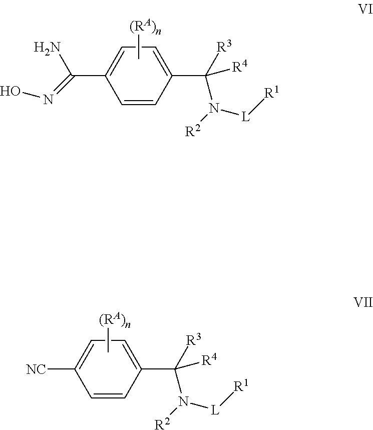
wherein the variables R^{A}, n, L, R¹, R², R³ and R⁴ are as defined in claim 14 for compounds of the formula I.

22. An agrochemical composition, which comprises an auxiliary and at least one compound of claim 14, or an N-oxide, or an agriculturally acceptable salt thereof.

23. An agrochemical composition according to claim 22, wherein the auxiliary is selected from the group of ionic or non-ionic surfactants.

24. An agrochemical composition according to claim 22, further comprising seed, wherein the amount of the compound of the formula I, or an N-oxide, or an agriculturally acceptable salt thereof, is from 0.1 g to 10 kg per 100 kg of seed.

25. A method for combating phytopathogenic harmful fungi, which process comprises treating the fungi, the plants, the soil or seeds to be protected against fungal attack, with an effective amount of at least one compound of formula I, or an N-oxide, or an agriculturally acceptable salt thereof, as defined in claim 14.

26. The method of claim 25, wherein n is 0 or 1; and wherein R^{A} is fluorine, chlorine, methyl or ethyl.

27. The method of claim 25, wherein R³ and R⁴ are independently selected from hydrogen, halogen, C₁-C₆-alkyl or C₁-C₆-haloalkyl.

28. The method of claim 25, wherein L is —S(═O)₂—.

29: The method of claim 25, wherein R² is hydrogen, C₁-C₆-alkyl, C₂-C₆-alkenyl or C₃-C₈-cycloalkyl.

30. The method of claim 25, wherein R² is hydrogen, C₁-C₆-alkyl, C₁-C₆-alkenyl, C₃-C₈-cycloalkyl, C₃-C₈-cycloalkyl-C₁-C₄-alkyl; preferably hydrogen, methyl, ethyl, iso-propyl, cyclopropyl, CH₂-cyclopropyl or allyl; and R¹ is C₃-C₈-cycloalkyl-C₁-C₂-alkyl; and wherein any of the alkyl or cycloalkyl group in R¹ are unsubstituted or substituted with 1, 2, 3 or 4 or up to the maximum possible number of radicals selected from the group consisting of chlorine and fluorine.

31. The method of claim 25, wherein R² is hydrogen, C₁-C₆-alkyl, C₂-C₆-alkenyl or C₃-C₈-cycloalkyl; and R¹ is C₁-C₆-alkyl; and wherein the alkyl group in R¹ is substituted with 1, 2, 3 or up to the maximum possible number of atoms selected from the group consisting of fluorine and chlorine.
